(19) United States
(12) Patent Application Publication
(10) Pub. No.: US 2025/0188074 A1
(43) Pub. Date: Jun. 12, 2025

(54) PTPN11 INHIBITORS

(71) Applicant: NAVIRE PHARMA, INC., Palo Alto, CA (US)

(72) Inventors: Philip JONES, Houston, TX (US); Barbara CZAKO, Bellaire, TX (US); Christopher L. CARROLL, Houston, TX (US); Pijus MANDAL, Sugarland, TX (US); Jason CROSS, Pearland, TX (US)

(21) Appl. No.: 18/799,303

(22) Filed: Aug. 9, 2024

Related U.S. Application Data
(63) Continuation of application No. 18/587,790, filed on Feb. 26, 2024, now abandoned.
Application No. 18/587,790 is a continuation of application No. 17/380,943, filed on Jul. 20, 2021, now Pat. No. 11,945,815.
Application No. 17/380,943 is a continuation of application No. 16/536,923, filed on Aug. 9, 2019, now Pat. No. 11,104,675.
(60) Provisional application No. 62/773,921, filed on Nov. 30, 2018.
(60) Provisional application No. 62/717,588, filed on Aug. 10, 2018.

(51) Int. Cl. C07D 471/04; A61P 35/00; C07D 491/107

(52) U.S. Cl. CPC C07D 471/04; A61P 35/00; C07D 491/107

(57) ABSTRACT

The present invention relates to compounds which are useful as inhibitors of PTPN11 for the treatment or prevention of cancer and other PTP-mediated diseases. Disclosed herein are new compounds and compounds based on pyrazolopyrazines and their application as pharmaceuticals for the treatment of disease.

### CROSS-REFERENCES TO RELATED APPLICATIONS

This application is a continuation of U.S. application Ser. No. 18/587,790, filed Feb. 26, 2024, which is a continuation of U.S. patent application Ser. No. 17/380,943, filed Jul. 20, 2021, now U.S. Pat. No. 11,945,815, which is a continuation of U.S. patent application Ser. No. 16/536,923, filed Aug. 9, 2019, now U.S. Pat. No. 11,104,675, which claims benefit of priority to U.S. Provisional Application No. 62/717,588, filed Aug. 10, 2018 and U.S. Provisional Application No. 62/773,921, filed Nov. 30, 2018, each of which is incorporated herein by reference in its entirety and for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Disclosed herein are new compounds and compounds based on pyrimidinones and their application as pharmaceuticals for the treatment of disease. Methods of inhibition of PTPN11 (SHP2) activity in a human or animal subject are also provided for the treatment diseases such as cancer, including leukemia and melanoma, and cancers of the breast, lung, and colon.

Tyrosyl phosphorylation regulates human cellular processes from cell differentiation to growth and apoptosis, and others. Tyrosyl phosphorylation is regulated by protein-tyrosine kinases (PTK) and protein-tyrosine phosphatases (PTP). The breakdown of regulation governed by PTK and PTP activity is thought to lead to cancer. PTK inhibitors have been developed as potential cancer therapeutic agents. Recent studies disclose a role for PTPs in cellular regulation as well. (A J Barr et al. Cell 2009, 136, 352-363. J N Andersen et al Mol. Cell. Biol. 2001, 21, 7117-7136).

Protein-tyrosine phosphatase non-receptor type 11 (PTPN11, also known as Src Homology-2 phosphatase (SHP2)) is a non-receptor protein tyrosine phosphatase encoded by the PTPN11 gene. This PTP contains two tandem Src homology-2 (SH2) domains, which function as phospho-tyrosine binding domains, a catalytic domain, and a C-terminal tail. In the basal state the protein typically exists in an inactive, self-inhibited conformation with the N-terminal SH2 domain blocking the active site. When stimulated by signal transduction mediated by cytokines and growth factor binding of phosphorylated proteins to the SH2 domains the auto-inhibition is relieved, this makes the active site available for dephosphorylation of PTPN11 substrates (M G Mohl, B G Neel, Curr. Opin. Genetics Dev. 2007, 17, 23-30. K S Grossmann, Adv. Cancer Res. 2010, 106, 53-89. W. Q. Huang et. al. Curr. Cancer Drug Targets 2014, 14, 567-588. C. Gordon et. al. Cancer Metastasis Rev. 2008, 27, 179-192).

Germ-line and somatic mutations in PTPN11 have been reported in several human diseases resulting in gain-of-function in the catalytic activity, including Noonan Syndrome and Leopard Syndrome; as well as multiple cancers such as juvenile myelomonocytic leukemia, neuroblastoma, myelodysplastic syndrome, B cell acute lymphoblastic leukemia/lymphoma, melanoma, acute myeloid leukemia and cancers of the breast, lung and colon (M G Mohl, B G Neel, Curr. Opin. Genetics Dev. 2007, 17, 23-30). Recent studies have demonstrated that single PTPN11 mutations are able to induce Noonan syndrome, JMML-like myeloproliferative disease and acute leukemia in mice. These mutations disrupt the auto-inhibition between the N—SH2 domains and the catalytic site allowing constitutive access of substrates to the catalytic site of the enzyme (E. Darian et al, Proteins, 2011, 79, 1573-1588. Z-H Yu et al, JBC, 2013, 288, 10472, W Qiu et al BMC Struct. Biol. 2014, 14, 10).

PTPN11 is widely expressed in most tissues and plays a regulatory role in various cell signaling events that are important for a diversity of cell functions that includes proliferation, differentiation, cell cycle maintenance, EMT transition, mitogenic activation, metabolic control, transcription regulation, and cell migration, through multiple signaling pathways including the Ras-MAPK, the JAK-STAT or the PI3K-AKT pathways (Tajan, M. et. al. Eur. J. Medical Genetics, 2015, 58, 509-525. Prahallad, A. et. al. Cell Reports, 2015, 12, 1978-1985).

Additionally there is growing evidence that PTPN11/SHP2 is implicated in immune evasion during tumorigenesis, and hence a SHP2 inhibitor could stimulate the immune response in cancer patients (Cancer Res. 2015 Feb. 1; 75(3):508-18. T Yokosuka T, J Exp Med. 2012, 209(6), 1201. S Amarnath Sci Transl Med. 2011, 3, 111ra120. T Okazaki, PNAS 2001, 98:24, 13866-71).

Novel compounds and pharmaceutical compositions, certain of which have been found to inhibit PTPN11 (SHP2) have been discovered, together with methods of synthesizing and using the compounds including methods for the treatment of PTP-mediated diseases in a patient by administering the compounds.

### BRIEF SUMMARY OF THE INVENTION

In certain embodiments of the present invention, compounds are represented by Formula I:

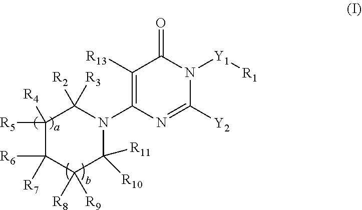

or a salt or tautomer thereof, wherein the subscripts a and b, Y₁, Y₂, and R₁, R₂, R₃, R₄, R₅, R₆, R₇, R₈, R₉, R₁₀, R₁₁, and R₁₃ are as provided herein.

In certain embodiments, the present invention provides a pharmaceutical composition including a compound having Formula I, together with a pharmaceutically acceptable carrier.

In certain embodiments, the present invention provides methods of inhibition of PTPN11 (SHP2) activity in a human or animal subject for the treatment diseases such as cancer, including leukemia and melanoma, and cancers of the breast, lung, and colon.

### BRIEF DESCRIPTION OF THE DRAWINGS

FIG. 1 shows a dose-dependent tumor growth inhibition by 6-((3S,4S)-4-amino-3-methyl-2-oxa-8-azaspiro[4.5]decan-8-yl)-3-(Rₐ)-(2,3-dichlorophenyl)-2-methylpyrimidin-4(3H)-one (Example 2b) in the KYSE520 xenograft model.

FIG. 2 shows a dose-dependent tumor growth inhibition by 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one (Example 10b) in the KYSE520 xenograft model.

### DETAILED DESCRIPTION OF THE INVENTION

### I. General

Certain compounds of Formula I disclosed herein may possess useful PTPN11 inhibiting activity, and may be used in the treatment or prophylaxis of a disease or condition in which PTPN11 plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting PTPN11. Other embodiments provide methods for treating a PTPN11-mediated disorder in a patient in need of such treatment, comprising administering to said patient a therapeutically effective amount of a compound or composition according to the present invention. Also provided is the use of certain compounds disclosed herein for use in the manufacture of a medicament for the treatment of a disease or condition ameliorated by the inhibition of PTPN11. In particular, the PTPN11-mediated disease is cancer.

### II. Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkynyl” refers to either a straight chain or branched hydrocarbon having at least 2 carbon atoms and at least one triple bond and having the number of carbon atom indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups are optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The terms “amido” and “carbamoyl,” as used herein, alone or in combination, refer to an amino group as described below attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes—C(O)NH₂, C₁₋₄alkylamido, and di(C₁₋₄alkyl)amido. The term “C₁₋₄alkylamido”, as used herein, refers to —C(O)NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which is optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term aryloxy as used herein, alone or in combination, refers to an aryl group attached to the parent molecular moiety through an oxy.

The term “carbamate,” as used herein, alone or in combination, refers to an ester of carbamic acid (—NHCOO—) which may be attached to the parent molecular moiety from either the nitrogen or acid end, and which is optionally substituted as defined herein.

The term “O-carbamyl” as used herein, alone or in combination, refers to a —OC(O)NRR′, group - with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a

“\*MERGEFORMAT\*MERGEFORMAT —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “iminohydroxy,” as used herein, alone or in combination, refers to ═N(OH) and ═N—O—.

The term “lower amino,” as used herein, alone or in combination, refers to

“\*MERGEFORMAT\*MERGEFORMAT —NRR′, wherein R and R are independently selected from hydrogen and lower alkyl, either of which is optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), S(O)m. Unless specifically prohibited, a ring is optionally substituted.

The terms “sulfonate,” “sulfonic acid,” and “sulfonic,” as used herein, alone or in combination, refer to the —SO₃H group and its anion as the sulfonic acid is used in salt formation.

The term “sulfanyl,” as used herein, alone or in combination, refers to —S—.

The term “sulfinyl,” as used herein, alone or in combination, refers to

“\*MERGEFORMAT\*MERGEFORMAT —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The term “tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower haloalkyl, lower haloalkenyl, lower haloalkynyl, lower perhaloalkyl, lower perhaloalkoxy, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from hydrogen, alkyl, cycloalkyl, heteroalkyl, aryl, heteroaryl and heterocycloalkyl, any of which is optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

Conformational isomers exist in the compounds disclosed herein. When R₁ is aryl or heteroaryl in the formula:

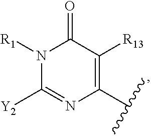

the aryl or heteroaryl group can orient in different conformations in relation to the pyrimidinone moiety, as represented by:

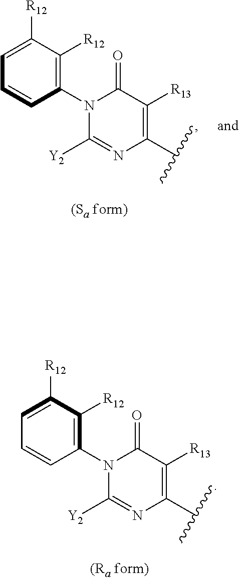

These forms are designated by the symbols “Sₐ” or “Rₐ”, depending on the conformation of the aryl or heteroaryl group in relation to the pyrimidinone moeity. Examples of “Sₐ” and “Rₐ” forms can be found in Examples 1-20.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PTPN11 inhibitor” is used herein to refer to a compound that exhibits an IC₅₀ with respect to PTPN11 activity of no more than about 10 μM and more typically not more than about 50 μM, as measured in the PTPN11 assay described generally herein. “IC₅₀” is that concentration of inhibitor which reduces the activity of an enzyme (e.g., PTPN11) to half-maximal level. Certain compounds disclosed herein have been discovered to exhibit inhibition against PTPN11. In certain embodiments, compounds will exhibit an IC₅₀ with respect to PTPN11 of no more than about 5 μM; in further embodiments, compounds will exhibit an IC₅₀ with respect to PTPN11 of no more than about 10 μM; in yet further embodiments, compounds will exhibit an IC₅₀ with respect to PTPN11 of not more than about 1 μM; in yet further embodiments, compounds will exhibit an IC₅₀ with respect to PTPN11 of not more than about 200 nM, as measured in the PTPN11 assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

### III. Compounds

In one aspect, the present invention provides a compound represented by Formula I:

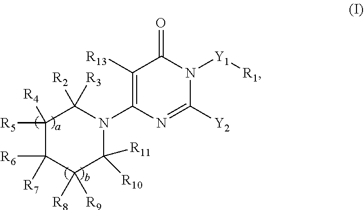

or a salt, ester, or prodrug thereof, wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—,
C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one to five groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O)ₘ;
subscript m is 0, 1, or 2;
said saturated or unsaturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices elected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, —NH—NHR₁₉, —NHR₁₉, —OR₁₉, —NHC(O)R₁₉, —NHC(O)NHR₁₉, —NHS(O)₂NHR₁₉, —NHS(O)₂R₁₉, —C(O)OR₁₉, —C(O)NR₁₉R₂₀, —C(O)NH(CH₂)_{q}OH, —C(O)NH(CH₂)_{q}R₂₁, —C(O)R₂₁, —NH₂, —OH, —S(O)₂NR₁₉R₂₀,
C₃₋₈cycloalkyl, aryl, heterocyclyl having 1-5 heteroatoms as ring vertices selected from N, O, S and P, heteroaryl having 1-5 heteroatoms as ring vertices selected from N, O, S and P; wherein the subscript q is an integer of from 0 to 6; and wherein aryl, heteroaryl, heterocyclyl and cycloalkyl are substituted with 0 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and a 5-10 membered heteroaryl, each of which is optionally substituted by one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆alkyl, C₁₋₆haloalkyl, C₁₋₆hydroxyalkyl, C₂₋₆alkenyl, C₂₋₆alkynyl and C₃₋₆cycloalkyl.

In some embodiments, the present invention provides a compound represented by Formula I:

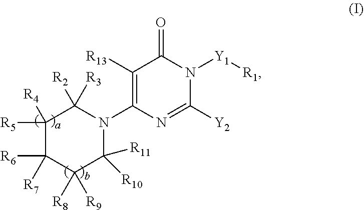

or a salt, ester, or prodrug thereof, wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—, C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is optionally substituted with one to five groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O)ₘ;
subscript m is 0, 1, or 2;
said saturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices selected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, —NH—NHR₁₉, —NHR₁₉, —OR₁₉, —NHC(O)R₁₉, —NHC(O)NHR₁₉, —NHS(O)₂NHR₁₉, —NHS(O)₂R₁₉, —C(O)OR₁₉, —C(O)NR₁₉R₂₀, —C(O)NH(CH₂)_{q}OH, —C(O)NH(CH₂)_{q}R₂₁, —C(O)R₂₁, —NH₂, —OH, —S(O)₂NR₁₉R₂₀, C₃₋₈cycloalkyl, aryl, heterocyclyl having 1-5 heteroatoms as ring vertices selected from N, O, S and P, heteroaryl having 1-5 heteroatoms as ring vertices selected from N, O, S and P; wherein the subscript q is an integer of from 0 to 6; and wherein aryl, heteroaryl, heterocyclyl and cycloalkyl are substituted with 0 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, each of which is optionally substituted by one or more groups independently selected from the group consisting of halo, hydroxy, cyano, and C₁₋₄alkyl;
R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆alkyl, C₁₋₆haloalkyl, C₁₋₆hydroxyalkyl, C₂₋₆alkenyl, C₂₋₆alkynyl and C₃₋₆cycloalkyl.

In some embodiments of Formula I, Y₁ is a direct bond. In some embodiments, Y₁ is CR₁₇R₁₈. In some embodiments, R₁₇ and R₁₈ are each independently selected from the group consisting of hydrogen, C₁₋₄alkyl, and CF₃. In some embodiments, R₁₇ and R₁₈ are each independently hydrogen or C₁₋₄alkyl. In some embodiments, Y₁ is —CH₂.

In some embodiments of Formula I, Y₂ is C₁₋₄alkyl. In some embodiments, Y₂ is methyl.

In some embodiments, the compound is represented by Formula Ia:

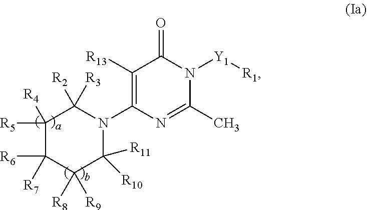

wherein the subscripts a and b, Y₁, R₁, R₂, R₃, R₄, R₅, R₆, R₇, R₈, R₉, R₁₀, R₁₁, and R₁₃ are as defined and described herein.

In some embodiments, the compound is represented by Formula Ib:

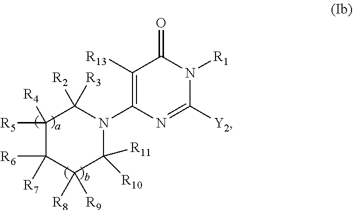

wherein the subscripts a and b, Y₂, R₁, R₂, R₃, R₄, R₅, R₆, R₇, R₈, R₉, R₁₀, R₁₁, and R₁₃ are as defined and described herein.

In some embodiments, the compound is represented by Formula Ic:

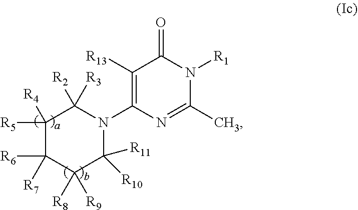

wherein the subscripts a and b, R₁, R₂, R₃, R₄, R₅, R₆, R₇, R₈, R₉, R₁₀, R₁₁, and R₁₃ are as defined and described herein.

In some embodiments of any one of Formulae I, Ia, Ib, and Ic, subscripts a and b are each 1.

In some embodiments of any one of Formulae I, Ia, Ib, and Ic, R₁₃ is selected from the group consisting of hydrogen, halo, C₁₋₆alkyl, C₁₋₆haloalkyl, C₁₋₆hydroxyalkyl,

“\*MERGEFORMAT\*MERGEFORMAT C₁₋₆dihydroxyalkyl, C₃₋₈cycloalkyl, 3- or 6-membered heterocyclyl having 1-3 heteroatoms as ring vertices selected from N, O and S; wherein heterocyclyl and cycloalkyl are substituted with 0 to 3 groups independently selected from the group consisting of C₁₋₄alkyl, —OH, —NH₂, —OR₂₁, halo, cyano and oxo. In some embodiments, R₁₃ is selected from the group consisting of hydrogen, halo, C₁₋₆alkyl, C₁₋₆haloalkyl, C₁₋₆hydroxyalkyl, C₁₋₆dihydroxyalkyl, and

“\*MERGEFORMAT\*MERGEFORMAT C₃₋₈cycloalkyl. In some embodiments, R₁₃ is selected from the group consisting of hydrogen, halo, C₁₋₆alkyl, and C₁₋₆ haloalkyl. In some embodiments, R₁₃ is selected from the group consisting of hydrogen, halo, C₁₋₄alkyl, and C₁₋₄haloalkyl. In some embodiments, R₁₃ is selected from the group consisting of —CH₂OH, CF₂OH, and —CHFOH. In some embodiments, R₁₃ is selected from the group consisting of hydrogen, Cl, Br, methyl, and CF₃. In some embodiments, R₁₃ is hydrogen. In some embodiments, R₁₃ is Cl. In some embodiments, R₁₃ is Br. In some embodiments, R₁₃ is methyl. In some embodiments, R₁₃ is CF₃.

In some embodiments of any one of Formulae I, Ia, Ib, and Ic, R₁ is selected from the group consisting of C₆₋₁₀aryl and a 5- to 9-membered heteroaryl group having 1 to 4 heteroatoms groups as ring vertices independently selected from N, C(O), O, and S; and is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy,

“\*MERGEFORMAT\*MERGEFORMAT C₁₋₄hydroxyalkyl, C₁₋₄haloalkyl, C₁₋₄ aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄.

In some embodiments of any one of Formulae I, Ia, Ib, and Ic, R₁ is selected from the group consisting of C₆₋₁₀aryl and a 5- to 9-membered heteroaryl group having 1 to 4 heteroatoms groups as ring vertices independently selected from N, C(O), O, and S; and is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy,

“\*MERGEFORMAT\*MERGEFORMAT C₁₋₄hydroxyalkyl, C₁₋₄haloalkyl, C₁₋₄ aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄; and R₁₄, R₁₅ and R₁₆ are each independently selected from the group consisting of hydrogen, C₁₋₄alkyl, C₃₋₈cycloalkyl,

“\*MERGEFORMAT\*MERGEFORMAT C₆₋₁₀aryl and 5-10 membered heteroaryl, each of which is optionally substituted by one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiments of any one of Formulae I, Ia, Ib, and Ic, R₁ is selected from the group consisting of C₆₋₁₀aryl and a 5- to 9-membered heteroaryl group having 1 to 4 heteroatoms groups as ring vertices independently selected from N, C(O), O, and S; and is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy,

“\*MERGEFORMAT\*MERGEFORMAT C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄; and R₁₄, R₁₅ and R₁₆ are each independently selected from the group consisting of hydrogen, C₁₋₄alkyl, C₃₋₈cycloalkyl,

“\*MERGEFORMAT\*MERGEFORMAT C₆₋₁₀aryl and 5-10 membered heteroaryl, each of which is optionally substituted by one or more groups independently selected from the group consisting of halo, hydroxy, cyano, and C₁₋₄alkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄ alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is selected from the group consisting of hydrogen, C₁₋₄alkyl, C₃₋₈cycloalkyl, C₆₋₁₀aryl and 5-10 membered heteroaryl, each of which is optionally substituted by one or more groups independently selected from the group consisting of amido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is selected from the group consisting of hydrogen, C₁₋₄alkyl, C₃₋₈cycloalkyl, C₆₋₁₀aryl and 5-10 membered heteroaryl, each of which is optionally substituted by one or more groups independently selected from the group consisting of halo, hydroxy, cyano, and C₁₋₄alkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄ alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is selected from the group consisting of C₆₋₁₀aryl and a 5-10 membered heteroaryl, each of which is optionally substituted by one or more groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy,

“\*MERGEFORMAT\*MERGEFORMAT C₁₋₄alkylamino and C₁₋₄aminoalkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is selected from the group consisting of C₆₋₁₀aryl and a 5-10 membered heteroaryl, each of which is optionally substituted by one or more groups independently selected from the group consisting of halo, hydroxy, cyano, and C₁₋₄alkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy,

“\*MERGEFORMAT\*MERGEFORMAT C₁₋₄haloalkyl, C₁₋₄haloalkoxy, C₁₋₄alkylamino and C₁₋₄aminoalkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄ alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is optionally substituted by one or more groups independently selected from the group consisting of halo, hydroxy, cyano, and C₁₋₄alkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄ alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is optionally substituted by one or more groups independently selected from the group consisting of C₁₋₄alkylamido, halo, hydroxy, cyano, and C₁₋₄alkyl.

In some embodiments of any one of Formulae I, Ia, Ib, and Ic, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; and R₁₄ is phenyl or a 5-6 membered heteroaryl having 1 to 4 heteroatoms as ring vertices independently selected from N, O, and S, each of which is optionally substituted by one or more groups independently selected from the group consisting of C₁₋₄alkylamido and C₁₋₄alkyl.

In some embodiments of any one of Formulae I, Ia, Ib, and Ic, R₁ is phenyl or a 5- to 6-member heteroaryl group having 1 to 4 heteroatoms or groups as ring vertices independently selected from N, C(O), O, and S; and is optionally substituted with 1, 2 or 3 R₁₂ groups independently selected from the group consisting of halo, hydroxy, amino, C₁₋₄ alkylamino,

“\*MERGEFORMAT\*MERGEFORMAT di(C₁₋₄alkyl)amino, C₁₋₄alkyl, C₁₋₄alkoxy, C₁₋₄haloalkyl, and C₁₋₄aminoalkyl.

In some embodiments of any one of Formulae I, Ia, Ib, and Ic, R₂, R₃, R₁₀, and R₁₁ are independently hydrogen or C₁₋₄alkyl. In certain embodiments, R₂, R₃, R₁₀, and Ru are each hydrogen.

In some embodiments of any one of Formulae I, Ia, Ib, and Ic, R₄, R₅, R₈, and R₉ are independently selected from the group consisting of hydrogen, C₁₋₄alkyl, C₁₋₄alkoxy, amino, hydroxy, C₃₋₈cycloalkyl, and C₁₋₄alkylamino. In certain embodiments, R₄, R₅, R₈, and R₉ are independently hydrogen or C₁₋₄alkyl. In certain embodiments, R₄, R₅, R₈, and R₉ are each hydrogen.

In some embodiments of any one of Formulae I, Ia, Ib, and Ic, R₁ is phenyl or a 5- to 6-member heteroaryl group having 1 to 4 heteroatoms or groups as ring vertices independently selected from N, C(O), O, and S; and is optionally substituted with 1, 2 or 3 R₁₂ groups independently selected from the group consisting of halo, hydroxy, amino, C₁₋₄alkylamino,

“\*MERGEFORMAT\*MERGEFORMAT di(C₁₋₄alkyl)amino, C₁₋₄alkyl, C₁₋₄alkoxy, C₁₋₄haloalkyl, and C₁₋₄aminoalkyl; and R₄, R₅, R₈, and R₉ are independently selected from the group consisting of hydrogen, C₁₋₄alkyl, C₁₋₄alkoxy, amino, hydroxy, C₃₋₆cycloalkyl, and C₁₋₄alkylamino.

In some embodiments of any one of Formulae I, Ia, Ib, and Ic, R₂, R₃, R₄, R₅, R₈, R₉, R₁₀ and R₁₁ are each hydrogen.

In some embodiments, the compound is represented by Formula II:

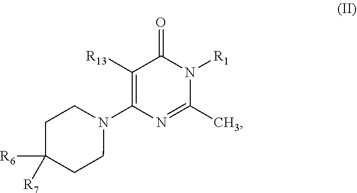

wherein R₁, R₆, R₇, and R₁₃ are as defined and described herein.

In some embodiments of any one of Formulae I, Ia-Ic, and II, R₆ is selected from the group consisting of amino, C₁₋₄aminoalkyl, and C₁₋₄alkylamino; and R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one to three substituents selected from the group consisting of amino, halo, hydroxy, cyano, trifluoromethyl, trifluoromethoxy, C₁₋₄alkyl, and C₁₋₄alkoxy. In some embodiments, R₆ is selected from the group consisting of amino,

“\*MERGEFORMAT\*MERGEFORMAT C₁₋₄aminoalkyl, and C₁₋₄alkylamino; and R₇ is selected from the group consisting of hydrogen, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of Formulae I, Ia-Ic, and II, R₆ is selected from the group consisting of amino, C₁₋₄aminoalkyl, and methylamino. In some embodiments, R₆ is amino or C₁₋₄aminoalkyl. In certain embodiments, R₆ is amino, aminomethyl, or methylamino. In certain embodiments, R₆ is amino or aminomethyl. In certain embodiments, R₆ is amino. In certain embodiments, R₆ is aminomethyl.

In some embodiments of any one of Formulae I, Ia-Ic, and II, R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is optionally substituted with one or two groups selected from the group consisting of amino, halo, hydroxy, cyano, trifluoromethyl, trifluoromethoxy,

“\*MERGEFORMAT\*MERGEFORMAT C₁₋₄alkyl, and C₁₋₄alkoxy. In some embodiments, R₇ is selected from the group consisting of hydroxy, C₁₋₄alkyl, C₁₋₄hydroxyalkyl, C₃₋₆cycloalkyl, phenyl, and 5- or 6-membered heteroaryl. In some embodiments, R₇ is hydroxy, C₁₋₄alkyl, or C₁₋₄hydroxyalkyl. In certain embodiments, R₇ is C₁₋₄alkyl. In certain embodiments, R₇ is methyl. In certain embodiments, R₇ is ethyl.

In some embodiments of any one of Formulae I, Ia-Ic, and II, R₆ is C₁₋₄ aminoalkyl; and R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is optionally substituted with one to three groups independently selected from the group consisting of amino, halo, hydroxy, cyano, trifluoromethyl, trifluoromethoxy, C₁₋₄alkyl, and C₁₋₄alkoxy.

In some embodiments of any one of Formulae I, Ia-Ic, and II, R₆ is aminomethyl; and R₇ is selected from the group consisting of hydroxy, C₁₋₄alkyl, C₁₋₄hydroxyalkyl, C₃₋₆cycloalkyl, phenyl, and 5- or 6-membered heteroaryl.

In some embodiments of any one of Formulae I, Ia-Ic, and II, R₆ is amino; and R₇ is selected from the group consisting of hydroxy, C₁₋₄alkyl, C₁₋₄hydroxyalkyl, C₃₋₆cycloalkyl, phenyl, and 5- or 6-membered heteroaryl, any of which is optionally substituted with one to three groups independently selected from the group consisting of amino, halo, hydroxy, cyano, trifluoromethyl, trifluoromethoxy, C₁₋₄alkyl, and C₁₋₄alkoxy.

In some embodiments of any one of Formulae I, Ia-Ic, and II, R₆ is amino; and R₇ is selected from the group consisting of hydroxy, C₁₋₄alkyl, C₁₋₄hydroxyalkyl, C₃₋₆cycloalkyl, phenyl, and 5- or 6-membered heteroaryl.

In some embodiments of any one of Formulae I, Ia-Ic, and II, R₆ is amino; and R₇ is C₁₋₄hydroxyalkyl. In some embodiments, R₆ is amino; and R₇ is hydroxymethyl. In some embodiments, R₆ is amino; and R₇ is C₁₋₄alkyl. In certain embodiments, R₆ is amino; and R₇ is methyl. In some embodiments, R₆ is amino; and R₇ is ethyl. In some embodiments, R₆ is aminomethyl; and R₇ is C₁₋₄alkyl. In certain embodiments, R₆ is aminomethyl; and R₇ is methyl. In some embodiments, R₆ is aminomethyl; and R₇ is ethyl.

In any of the above embodiments, the amido of R₇ may specifically be —C(O)NH₂.

In some embodiments of any one of Formulae I, Ia-Ic, and II, R₆ and R₇ together with the carbon atom to which they are both attached form a 3- to 7-membered saturated or unsaturated ring having 1 to 3 heteroatoms or groups as ring vertices independently selected from N, C(O), O, and S(O)ₘ, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₆ and R₇ together with the carbon atom to which they are both attached form a 4- to 6-membered saturated or unsaturated ring having 1 to 3 heteroatoms or groups as ring vertices independently selected from N, C(O), and O, and that is optionally substituted with one or two groups independently selected from the group consisting of amino, halo, hydroxy, C₁₋₄alkyl, C₁₋₄alkoxy,

“\*MERGEFORMAT\*MERGEFORMAT C₁₋₄alkylamino and C₁₋₄aminoalkyl.

In some embodiments of any one of Formulae I, Ia-Ic, and II, R₆ and R₇ together with the carbon atom to which they are both attached form a 3- to 7-membered saturated ring having 1 to 3 heteroatoms or groups as ring vertices independently selected from N, C(O), O, and S(O)ₘ, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₆ and R₇ together with the carbon atom to which they are both attached form a 4- to 6-membered saturated ring having 1 to 3 heteroatoms or groups as ring vertices independently selected from N, C(O), and O, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₆ and R₇ together with the carbon atom to which they are both attached form a 4- to 6-membered saturated ring having 1 to 3 heteroatoms or groups as ring vertices independently selected from N and O, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₆ and R₇ together with the carbon atom to which they are both attached form a 3- to 7-membered cycloalkyl ring that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₆ and R₇ together with the carbon atom to which they are both attached form a 4- to 6-membered cycloalkyl ring that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl; R₆ is selected from the group consisting of amino, C₁₋₄aminoalkyl, and C₁₋₄ alkylamino; and R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is optionally substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl; R₆ is amino or aminomethyl; and R₇ is selected from the group consisting of hydroxy, C₁₋₄alkyl, and C₁₋₄hydroxyalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl; and R₆ and R₇ together with the carbon atom to which they are both attached form a 3- to 7-membered saturated or unsaturated ring having 1 to 3 heteroatoms or groups as ring vertices independently selected from N, C(O), O, and S(O)ₘ, and that is optionally substituted with one or two groups independently selected from the group consisting of amino, halo, hydroxy, C₁₋₄alkyl, C₁₋₄alkoxy, C₁₋₄alkylamino and

“\*MERGEFORMAT\*MERGEFORMAT C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl; and R₆ and R₇ together with the carbon atom to which they are both attached form a 4- to 6-membered saturated or unsaturated ring having 1 to 3 heteroatoms or groups as ring vertices independently selected from N, C(O), and O, and that is optionally substituted with one or two groups independently selected from the group consisting of amino, halo, hydroxy, C₁₋₄alkyl, C₁₋₄alkoxy, C₁₋₄alkylamino and

“\*MERGEFORMAT\*MERGEFORMAT C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, C₁₋₄aminoalkyl, and OR₁₄; R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl; and R₆ and R₇ together with the carbon atom to which they are both attached form a 3- to 7-membered cycloalkyl ring that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, and C₁₋₄aminoalkyl; R₆ is selected from the group consisting of amino,

“\*MERGEFORMAT\*MERGEFORMAT C₁₋₄aminoalkyl, and C₁₋₄alkylamino; and R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is optionally substituted with one or two substituents selected from the group consisting of amino, halo, hydroxy, cyano, trifluoromethyl, trifluoromethoxy, C₁₋₄alkyl, and C₁₋₄alkoxy.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, and C₁₋₄aminoalkyl; R₆ is amino or aminomethyl; and R₇ is selected from the group consisting of hydroxy, C₁₋₄alkyl, and C₁₋₄hydroxyalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, and C₁₋₄aminoalkyl; and R₆ and R₇ together with the carbon atom to which they are both attached form a 3- to 7-membered saturated or unsaturated ring having 1 to 3 heteroatoms or groups as ring vertices independently selected from N, C(O), O, and S(O)ₘ, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, and C₁₋₄aminoalkyl; and R₆ and R₇ together with the carbon atom to which they are both attached form a 4- to 6-membered saturated or unsaturated ring having 1 to 3 heteroatoms or groups as ring vertices independently selected from N, C(O), and O, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl,

“\*MERGEFORMAT\*MERGEFORMAT C₁₋₄alkoxy, C₁₋₄haloalkyl, and C₁₋₄aminoalkyl; and R₆ and R₇ together with the carbon atom to which they are both attached form a 3- to 7-membered cycloalkyl ring that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

In some embodiments of any one of Formulae I, Ia-Ic, and II, R₁ is selected from the group consisting of phenyl, pyridyl, pyrimidinyl, pyrazolyl, pyrazinyl, pyridazinyl, and 1,2,4-triazinyl; and is optionally substituted with 1, 2, or 3 R₁₂, wherein each R₁₂ is as defined and described herein. In some embodiments, R₁ is selected from the group consisting of phenyl, pyridyl, pyrimidinyl, pyrazinyl, pyridazinyl, and 1,2,4-triazinyl; and is optionally substituted with 1, 2, or 3 R₁₂, wherein each R₁₂ is as defined and described herein. In some embodiments, R₁ is phenyl or pyridyl; and is optionally substituted with 1, 2, or 3 R₁₂, wherein each R₁₂ is as defined and described herein.

In some embodiments of any one of Formulae I, Ia-Ic, and II, R₁ is selected from the group consisting of phenyl, pyridyl, pyrimidinyl, pyrazinyl, pyridazinyl, and 1,2,4-triazinyl; and is optionally substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

In some embodiments of any one of Formulae I, Ia-Ic, and II, R₁ is selected from the group consisting of phenyl, pyridyl, pyrimidinyl, pyrazinyl, pyridazinyl, and 1,2,4-triazinyl; and is optionally substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of Formulae I, Ia-Ic, and II, R₁ is phenyl or pyridyl, each of which is optionally substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

In some embodiments of any one of Formulae I, Ia-Ic, and II, R₁ is phenyl or pyridyl, each of which is optionally substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of Formulae I, Ia-Ic, and II, R₁ is phenyl and is optionally substituted with 1-3 R₁₂, each of which is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄. In some embodiments, R₁ is phenyl and is optionally substituted with 1-3 R₁₂, each of which is independently selected from the group consisting of halo, hydroxy, amino, methylamino, dimethylamino, cyano, C₁₋₄alkyl, C₁₋₄haloalkyl, and

“\*MERGEFORMAT\*MERGEFORMAT C₁₋₄alkoxy. In some embodiments, R₁ is phenyl and is optionally substituted with 1-3 R₁₂, each of which is independently selected from the group consisting of halo, hydroxy, C₁₋₄alkyl, and

“\*MERGEFORMAT\*MERGEFORMAT C₁₋₄alkoxy.

In some embodiments of any one of Formulae I, Ia-Ic, and II, R₁ is pyridyl and is optionally substituted with 1-3 R₁₂, each of which is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄. In some embodiments, R₁ is pyridyl and is optionally substituted with 1-3 R₁₂, each of which is independently selected from the group consisting of halo, hydroxy, amino, methylamino, dimethylamino, cyano, C₁₋₄alkyl, C₁₋₄haloalkyl, and

“\*MERGEFORMAT\*MERGEFORMAT C₁₋₄alkoxy. In some embodiments, R₁ is pyridyl and is optionally substituted with 1-3 R₁₂, each of which is independently selected from the group consisting of halo, hydroxy, C₁₋₄alkyl, and

“\*MERGEFORMAT\*MERGEFORMAT C₁₋₄alkoxy.

In some embodiments of any one of Formulae I, Ia-Ic, and II, R₁ is selected from the group consisting of:

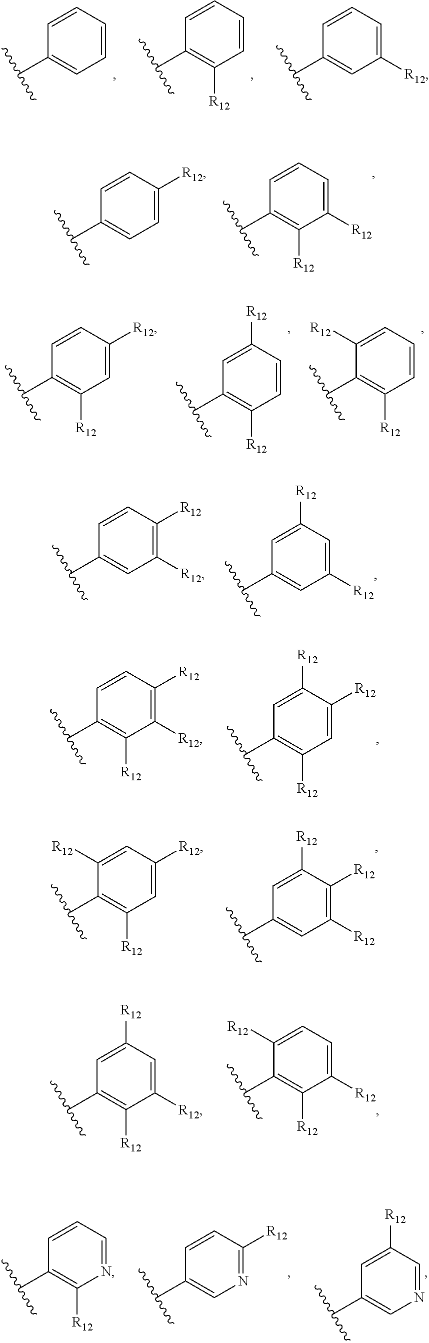

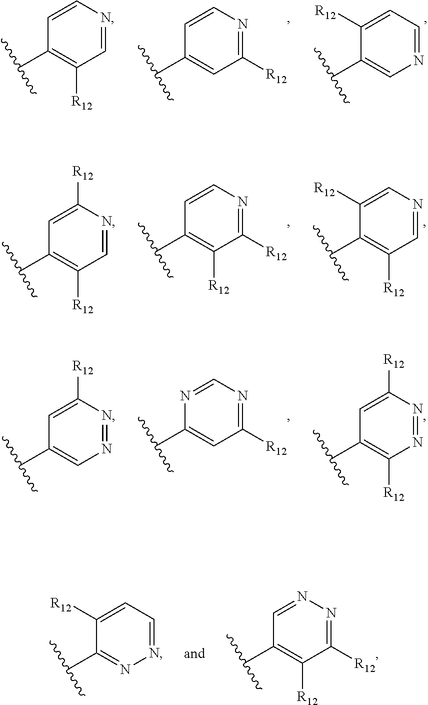

wherein each R₁₂ is as defined and described herein.

In some embodiments of any one of Formulae I, Ia-Ic, and II, R₁ is selected from the group consisting of:

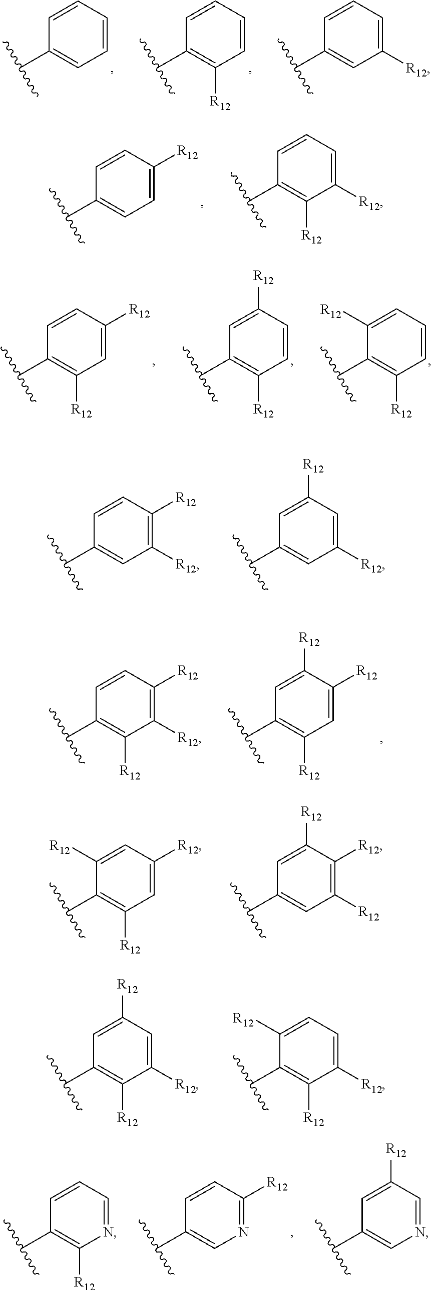

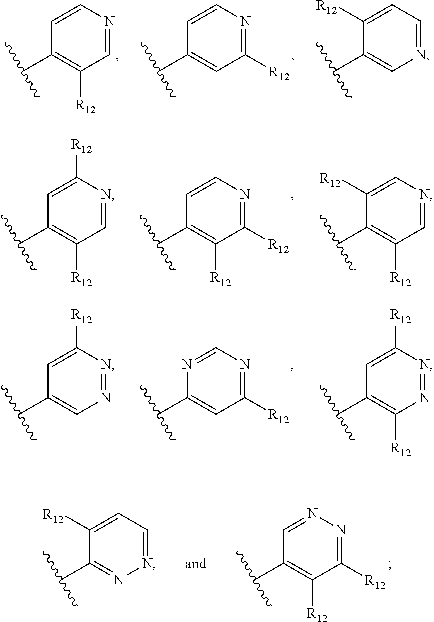

and each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄, wherein R₁₄ is as defined and described herein.

In some embodiments of any one of Formulae I, Ia-Ic, and II, R₁ is selected from the group consisting of:

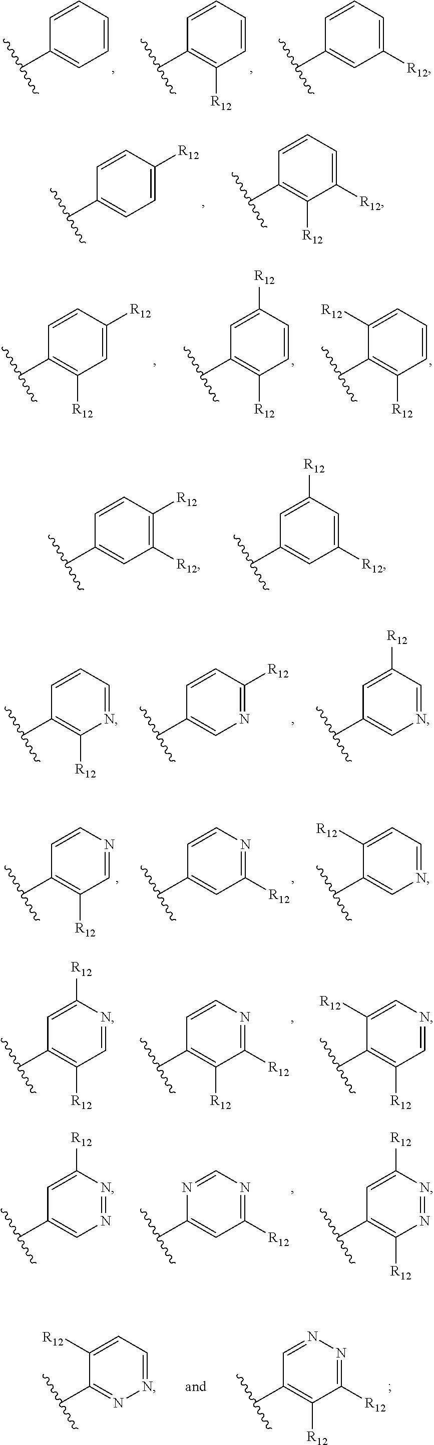

and each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl.

In some embodiments of any one of Formulae I, Ia-Ic, and II, R₁ is selected from the group consisting of:

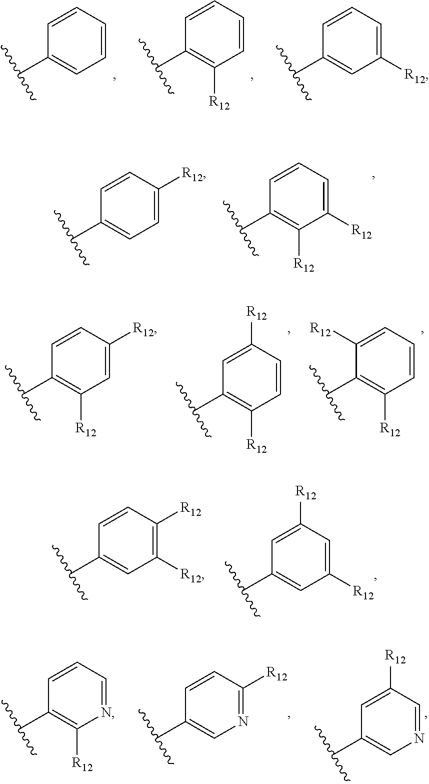

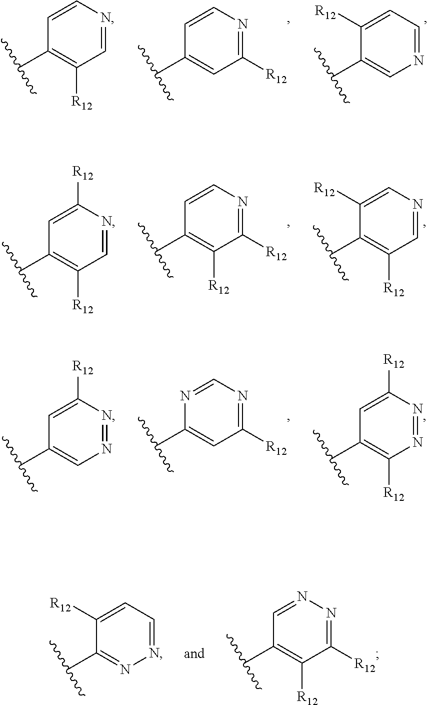

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of Formulae I, Ia-Ic, and II, R₁ is selected from the group consisting of:

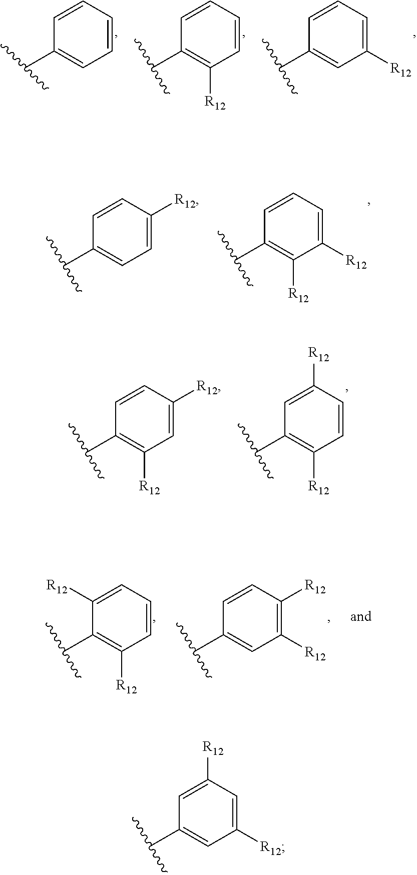

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of Formulae I, Ia-Ic, and I, R₅ is selected from the group consisting of:

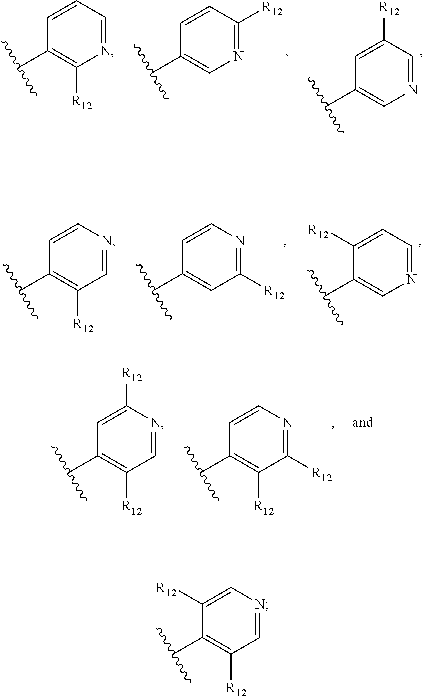

and each R₁₂ is independently selected from the group consisting of halo, hydroxy, amino, methylamino, dimethylamino, cyano, C₁₋₄alkyl, C₁₋₄haloalkyl, and C₁₋₄alkoxy.

In some embodiments of any one of Formulae I, Ia-Ic, and II, each R₁₂ is independently selected from the group consisting of halo, hydroxy, amino, C₁₋₄alkylamino, di(C₁₋₄ alkyl)amino, C₁₋₄alkyl, C₁₋₄alkoxy, C₁₋₄haloalkyl, C₁₋₄aminoalkyl, and OR₁₄. In some embodiments, each R₁₂ is independently selected from the group consisting of halo, C₁₋₄alkyl, C₁₋₄alkoxy, C₁₋₄haloalkyl, and OR₁₄. In some embodiments, each R₁₂ is independently selected from the group consisting of halo, hydroxy, amino, methylamino, dimethylamino, cyano, C₁₋₄alkyl, C₁₋₄haloalkyl, and

“\*MERGEFORMAT\*MERGEFORMAT C₁₋₄alkoxy. In some embodiments, each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃.

In some embodiments of any one of Formulae I, Ia-Ic, and II, each R₁₂ is independently selected from the group consisting of F, Cl, Br, CH₃, OCH₃, CF₃,

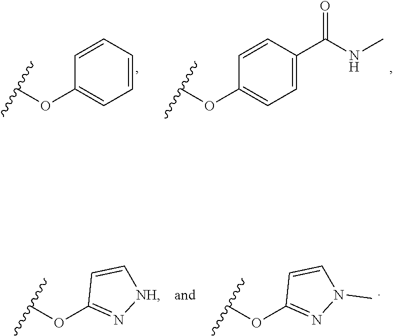

In some embodiments of any one of Formulae I, Ia-Ic, and II, R₁ is selected from the group consisting of:

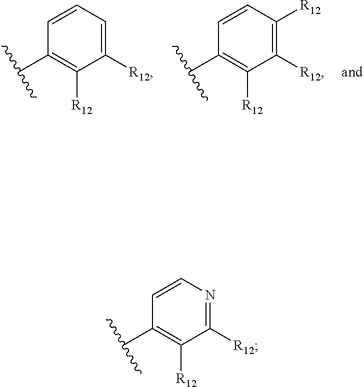

each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄, wherein R₁₄ is as defined and described herein.

In some embodiments of any one of Formulae I, Ia-Ic, and II, R₁ is represented by:

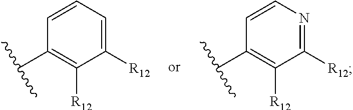

and each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃. In some embodiments, each R₁₂ is Cl.

In some embodiments of any one of Formulae I, Ia-Ic, and II, R₁ is represented by:

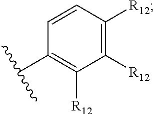

and each R₁₂ is independently selected from the group consisting of halo, C₁₋₄alkyl, C₁₋₄alkoxy, and C₁₋₄haloalkyl. In some embodiments, each R₁₂ is independently selected from the group consisting of F, Cl, Br, CH₃, OCH₃, and CF₃. In some embodiments, each R₁₂ is independently C₁ or Br.

In some embodiments of any one of Formulae I, Ia-Ic, and II, R₁₄ is independently selected from the group consisting of C₆₋₁₀aryl and a 5-10 membered heteroaryl, each of which is optionally substituted by one or more groups independently selected from the group consisting of C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl. In some embodiments, R₁₄ is independently selected from the group consisting of C₆₋₁₀aryl and a 5-10 membered heteroaryl, each of which is optionally substituted by one or two groups independently selected from the group consisting of C₁₋₄alkylamido, halo, hydroxy, cyano, and C₁₋₄alkyl. In some embodiments, R₁₄ is independently selected from the group consisting of C₆₋₁₀aryl and a 5-10 membered heteroaryl, each of which is optionally substituted by one or two groups independently selected from the group consisting of halo, hydroxy, cyano, and C₁₋₄alkyl.

In some embodiments of any one of Formulae I, Ia-Ic, and II, R₁₄ is independently phenyl or a 5-6 membered heteroaryl having 1 to 4 heteroatoms as ring vertices independently selected from N, O, and S, each of which is optionally substituted by one or more groups independently selected from the group consisting of C₁₋₄alkylamido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl. In some embodiments, R₁₄ is independently phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, halo, hydroxy, cyano, and C₁₋₄alkyl. In some embodiments, R₁₄ is independently phenyl or a 5-6 membered heteroaryl having 1 to 4 heteroatoms as ring vertices independently selected from N, O, and S, each of which is optionally substituted by one or two groups independently selected from the group consisting of halo, hydroxy, cyano, and C₁₋₄alkyl.

In some embodiments of any one of Formulae I, Ia-Ic, and II, R₁₄ is phenyl or pyrazolyl, each of which is optionally substituted by one or two groups independently selected from the group consisting of C₁₋₄alkylamido, halo, hydroxy, cyano, and C₁₋₄alkyl. In some embodiments, R₁₄ is phenyl, optionally substituted by one or two groups independently selected from the group consisting of C₁₋₄alkylamido, halo, hydroxy, cyano, and C₁₋₄alkyl. In some embodiments, R₁₄ is phenyl, substituted with C₁₋₄alkylamido. In some embodiments, R₁₄ is phenyl substituted with —C(O)NHMe. In some embodiments, R₁₄ is phenyl. In some embodiments, R₁₄ is pyrazolyl, optionally substituted by one or two groups independently selected from the group consisting of C₁₋₄alkylamido, halo, hydroxy, cyano, and C₁₋₄alkyl. In some embodiments, R₁₄ is pyrazolyl substituted with C₁₋₄alkyl. In some embodiments, R₁₄ is pyrazolyl substituted with methyl. In some embodiments, R₁₄ is N-methylpyrazolyl. In some embodiments, R₁₄ is pyrazolyl.

In some embodiments of any one of Formulae I, Ia-Ic, and II, R₁ is represented by:

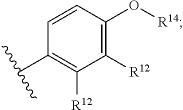

each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, halo, hydroxy, cyano, and C₁₋₄alkyl.

In some embodiments of any one of Formulae I, Ia-Ic, and II, R₁ is represented by:

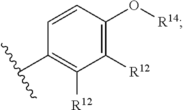

each R₁₂ is independently selected from the group consisting of halo, C₁₋₄alkyl, C₁₋₄alkoxy, C₁₋₄haloalkyl, and OR₁₄; and R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, halo, hydroxy, cyano, and C₁₋₄alkyl.

In some embodiments of any one of Formulae I, Ia-Ic, and II, R₁ is represented by:

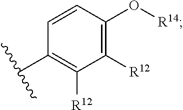

each R₁₂ is independently selected from the group consisting of halo, C₁₋₄alkyl, C₁₋₄alkoxy, C₁₋₄haloalkyl, and OR₁₄; and R₁₄ is selected from the group consisting of phenyl or pyrazolyl, each of which is optionally substituted by one or two groups independently selected from the group consisting of C₁₋₄alkylamido, halo, hydroxy, cyano, and C₁₋₄alkyl.

In some embodiments of any one of Formulae I, Ia-Ic, and II, R₁ is represented by:

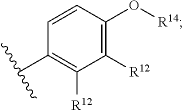

each R₁₂ is independently selected from the group consisting of halo, C₁₋₄alkyl, C₁₋₄alkoxy, C₁₋₄haloalkyl, and OR₁₄; and R₁₄ is selected from the group consisting of phenyl, phenyl substituted with C₁₋₄alkylamido, pyrazolyl, and pyrazolyl substituted with C₁₋₄alkyl. In some embodiments, each R₁₂ is independently selected from the group consisting of F, Cl, Br, CH₃, OCH₃, and CF₃; and R₁₄ is selected from the group consisting of phenyl, MeNHC(O)-phenyl, pyrazolyl, and N-methylpyrazolyl. In some embodiments, each R₁₂ is C₁; and R₁₄ is selected from the group consisting of phenyl, MeNHC(O)-phenyl, pyrazolyl, and N-methylpyrazolyl.

In certain embodiments, the compound is represented by Formula II:

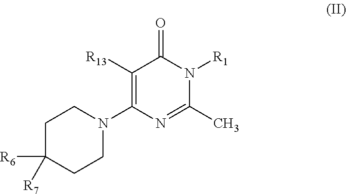

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₆ is selected from the group consisting of amino, C₁₋₄aminoalkyl, and C₁₋₄alkylamino;
R₇ is selected from the group consisting of hydrogen, cyano, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one to three substituents independently selected from the group consisting of amino, halo, hydroxy, cyano, trifluoromethyl, trifluoromethoxy, C₁₋₄alkyl, and C₁₋₄alkoxy;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl; and
R₁₄ is phenyl or pyrazolyl, each of which is optionally substituted by one or two groups independently selected from the group consisting of C₁₋₄alkylamido, halo, hydroxy, cyano, and C₁₋₄alkyl.

In certain embodiments, the compound is represented by Formula III:

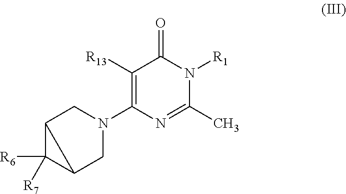

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₆ is selected from the group consisting of amino, C₁₋₄aminoalkyl, and C₁₋₄alkylamino;
R₇ is selected from the group consisting of hydrogen, cyano, amido, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one to three substituents independently selected from the group consisting of amino, halo, hydroxy, cyano, trifluoromethyl, trifluoromethoxy, C₁₋₄alkyl, and C₁₋₄alkoxy;
R₁₃ is selected from the group consisting of hydrogen, halo, cyano, C₁₋₆alkyl, C₁₋₆haloalkyl, C₁₋₆hydroxyalkyl, C₁₋₆ dihydroxyalkyl, and C₃₋₈cycloalkyl; and
R₁₄ is phenyl or pyrazolyl, each of which is optionally substituted by one or two groups independently selected from the group consisting of C₁₋₄alkylamido, halo, hydroxy, cyano, and C₁₋₄alkyl.

In certain embodiments, the compound is represented by Formula IV:

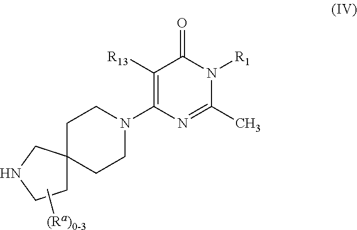

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄;
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In certain embodiments, the compound is represented by Formula V:

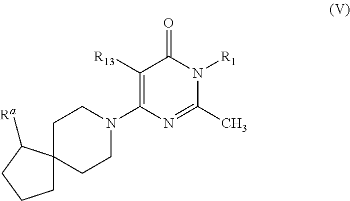

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
R^{a} is selected from the group consisting of amino, halo, hydroxy, cyano, trifluoromethyl, trifluoromethoxy, C₁₋₄alkyl, and C₁₋₄alkoxy.

In certain embodiments, the compound is represented by Formula VI:

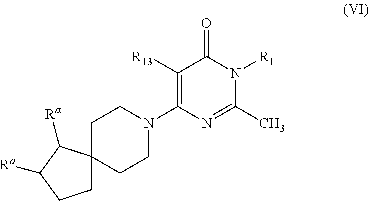

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by Formula VII:

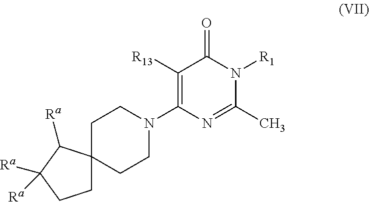

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by Formula VIII:

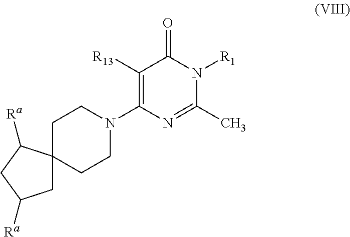

or a salt, ester or prodrug thereof, wherein:
R₁ is phenyl or pyridyl, each of which is substituted with 0 to 3 R₁₂;
each R₁ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, trifluoromethyl, trifluoromethoxy, C₁₋₄alkyl, and C₁₋₄alkoxy.

In certain embodiments, the compound is represented by Formula IX:

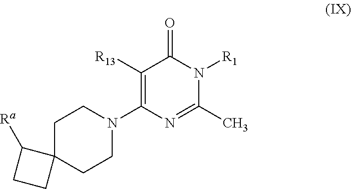

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
R^{a} is selected from the group consisting of amino, halo, hydroxy, cyano, trifluoromethyl, trifluoromethoxy, C₁₋₄alkyl, and C₁₋₄alkoxy.

In certain embodiments, the compound is represented by Formula X:

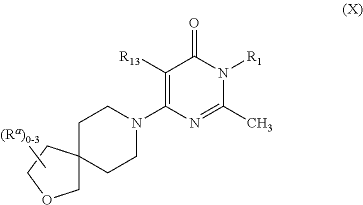

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In certain embodiments, the compound is represented by Formula XI:

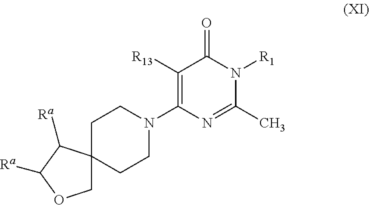

or a salt, ester or prodrug thereof, wherein:
R₁ is phenyl or pyridyl, each of which is substituted with 0 to 3 R₁₂;
each R₁₂ is independently selected from the group consisting of halo, hydroxy, C₁₋₄alkyl, C₁₋₄alkoxy, C₁₋₄haloalkyl, and OR₁₄; and
R₁₃ is selected from the group consisting of hydrogen, halo, cyano, C₁₋₆alkyl, C₁₋₆haloalkyl, C₁₋₆hydroxyalkyl, C₁₋₆dihydroxyalkyl, and C₃₋₈cycloalkyl;
R₁₄ is phenyl or pyrazolyl, each of which is optionally substituted by one or two groups independently selected from the group consisting of C₁₋₄alkylamido, halo, hydroxy, cyano, and C₁₋₄alkyl; and
each R^{a} is independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl.

In some embodiment of any one of Formulae II-XI, R₁, R₆, R₇, R₁₂, R₁₃, and R₁₄ may have the meanings set forth in any one or more of the selected embodiments noted above.

In some embodiments of any one of Formulae II-XI, R₁₃ is selected from the group consisting of hydrogen, halo, C₁₋₆alkyl, C₁₋₆haloalkyl, C₁₋₆hydroxyalkyl, C₁₋₆dihydroxyalkyl, and C₃₋₈cycloalkyl. In some embodiments, R₁₃ is selected from the group consisting of hydrogen, halo, C₁₋₆alkyl, and C₁₋₆haloalkyl. In some embodiments, R₁₃ is selected from the group consisting of hydrogen, halo, C₁₋₄alkyl, and C₁₋₄haloalkyl. In some embodiments, R₁₃ is selected from the group consisting of hydrogen, Cl, Br, methyl, and CF₃. In some embodiments, R₁₃ is hydrogen. In some embodiments, R₁₃ is Cl. In some embodiments, R₁₃ is Br. In some embodiments, R₁₃ is methyl. In some embodiments, R₁₃ is CF₃.

In some embodiments of any one of Formulae II-XI, R₁ is phenyl or pyridyl, each of which is substituted with 1 to 3 R₁₂. In some embodiments, R₁ is phenyl or pyridyl, each of which is substituted with 2 or 3 R₁₂. In some embodiments, R₁ is phenyl substituted with 2 or 3 R₁₂. In some embodiments, R₁ is phenyl substituted with 2 R₁₂. In some embodiments, R₁ is phenyl substituted with 3 R₁₂. In some embodiments, R₁ is pyridyl substituted with 2 R₁₂.

In some embodiments of any one of Formulae II-XI, each R₁₂ is independently selected from the group consisting of halo, C₁₋₄alkyl, C₁₋₄alkoxy, C₁₋₄haloalkyl, and OR₁₄. In some embodiments, each R₁₂ is independently selected from the group consisting of halo, hydroxy, amino, methylamino, dimethylamino, cyano, C₁₋₄alkyl, C₁₋₄haloalkyl, and C₁₋₄ alkoxy. In some embodiments, each R₁₂ is independently selected from the group consisting of halo, C₁₋₄alkyl,

“\*MERGEFORMAT\*MERGEFORMAT C₁₋₄alkoxy, and C₁₋₄haloalkyl. In some embodiments, each R₁₂ is independently selected from the group consisting of F, Cl, Br, CH₃, OCH₃, and CF₃.

In some embodiments of any one of Formulae II-XI, each R₁₂ is independently selected from the group consisting of F, Cl, Br, CH₃, OCH₃, CF₃,

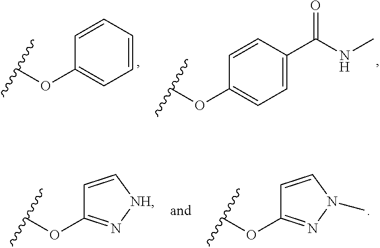

In some embodiments of any one of Formulae II-XI, R₁ is phenyl substituted with 2 R₁₂; and each R₁₂ is independently selected from the group consisting of F, Cl, Br, CH₃, OCH₃, and CF₃. In some embodiments, R₁ is phenyl substituted with 2 R₁₂; and each R₁₂ is Cl.

In some embodiments of any one of Formulae II-XI, R₁ is phenyl substituted with 3 R₁₂; and each R₁₂ is independently selected from the group consisting of F, Cl, Br, CH₃, OCH₃, CF₃,

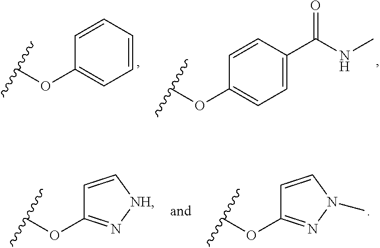

In some embodiments of any one of Formulae II-XI, R₁ is phenyl substituted with 3 R₁₂; the first and second R₁₂ are each C₁; and the third R₁₂ is Br. In some embodiments, R₁ is phenyl substituted with 3 R₁₂; the first and second R₁₂ are each C₁; and the third R₁₂ is selected from the group consisting of:

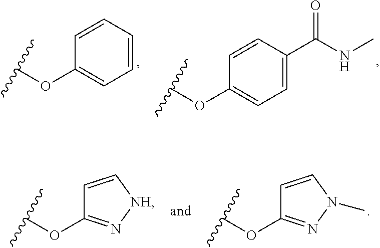

In some embodiments of any one of Formulae II-XI, In some embodiments, R₁₄ is phenyl, optionally substituted by one or two groups independently selected from the group consisting of C₁₋₄alkylamido, halo, hydroxy, cyano, and C₁₋₄alkyl. In some embodiments, R₁₄ is phenyl, substituted with C₁₋₄alkylamido. In some embodiments, R₁₄ is phenyl substituted with

“\*MERGEFORMAT\*MERGEFORMAT —C(O)NHMe. In some embodiments, R₁₄ is phenyl. In some embodiments, R₁₄ is pyrazolyl, optionally substituted by one or two groups independently selected from the group consisting of C₁₋₄alkylamido, halo, hydroxy, cyano, and C₁₋₄alkyl. In some embodiments, R₁₄ is pyrazolyl substituted with C₁₋₄alkyl. In some embodiments, R₁₄ is pyrazolyl substituted with methyl. In some embodiments, R₁₄ is N-methylpyrazolyl. In some embodiments, R₁₄ is pyrazolyl.

In some embodiments of Formula II or III, R₆ is amino or C₁₋₄aminoalkyl. In certain embodiments, R₆ is amino or aminomethyl. In certain embodiments, R₆ is amino. In certain embodiments, R₆ is aminomethyl.

In some embodiments of Formula II or III, R₇ is hydroxy, C₁₋₄alkyl, or C₁₋₄hydroxyalkyl. In certain embodiments, R₇ is C₁₋₄alkyl. In certain embodiments, R₇ is methyl. In certain embodiments, R₇ is ethyl.

In some embodiments of Formula II or III, R₆ is amino; and R₇ is C₁₋₄ alkyl. In certain embodiments, R₆ is amino; and R₇ is methyl. In some embodiments, R₆ is amino; and R₇ is ethyl. In some embodiments, R₆ is aminomethyl; and R₇ is C₁₋₄alkyl. In certain embodiments, R₆ is aminomethyl; and R₇ is methyl.

In some embodiments of any one of Formulae IV-XI, each R^{a} is independently selected from the group consisting of amino, halo, hydroxy, cyano, C₁₋₄alkyl, C₁₋₄alkoxy, and

“\*MERGEFORMAT\*MERGEFORMAT C₁₋₄haloalkyl. In some embodiments, each R^{a} is independently amino or C₁₋₄alkyl. In some embodiments, each R^{a} is independently amino or methyl.

In some embodiments, the compound is represented by the formula selected from the group consisting of:

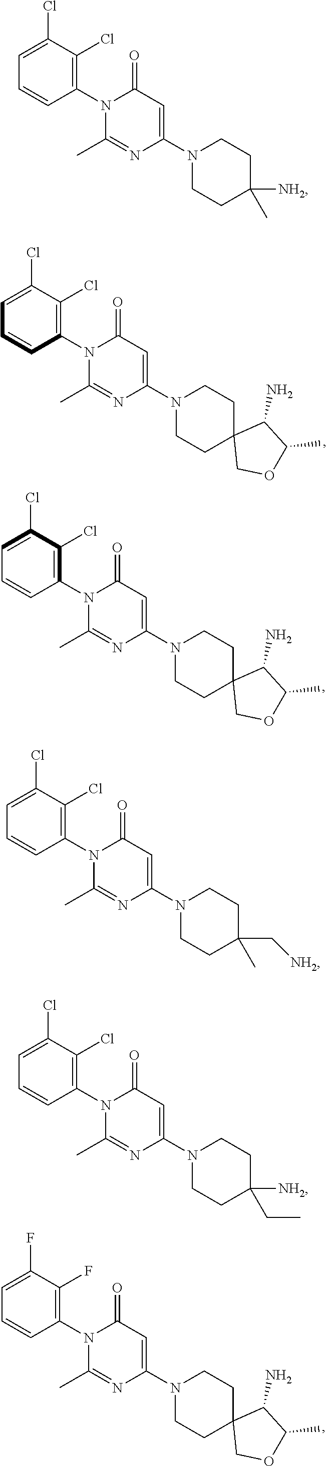

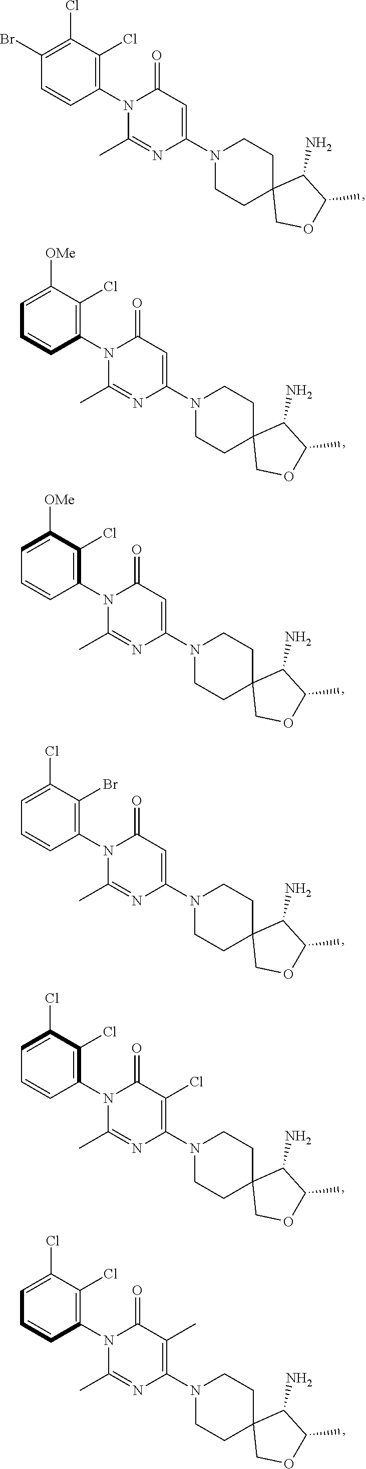

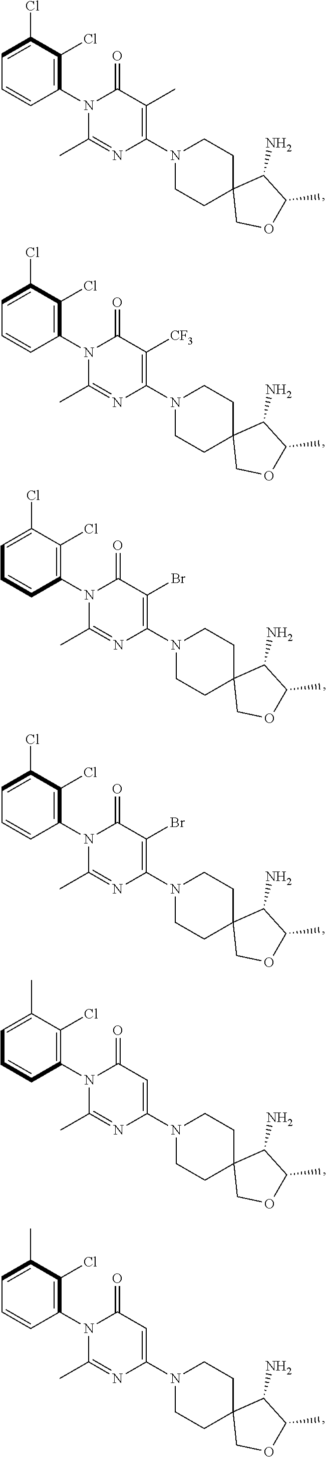

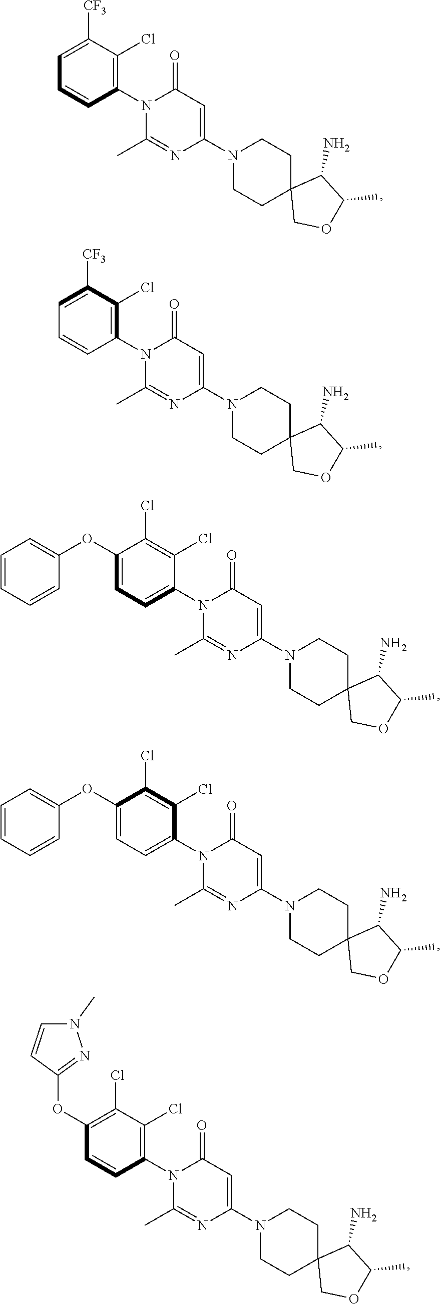

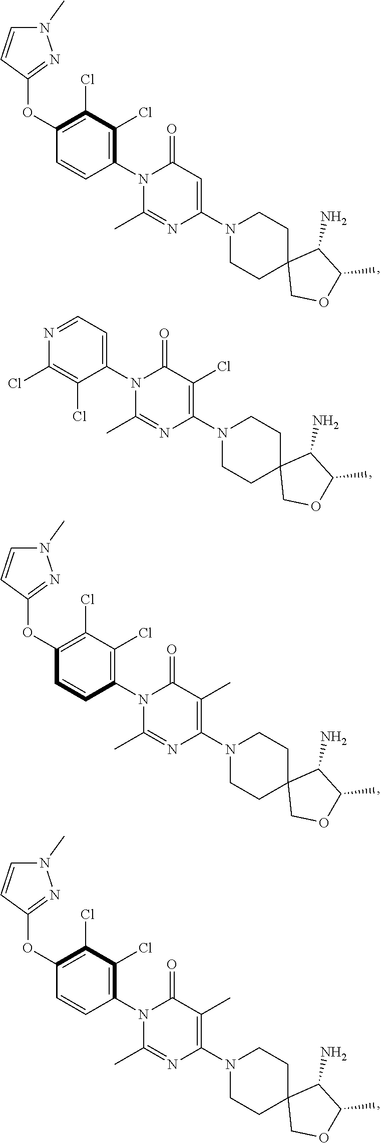

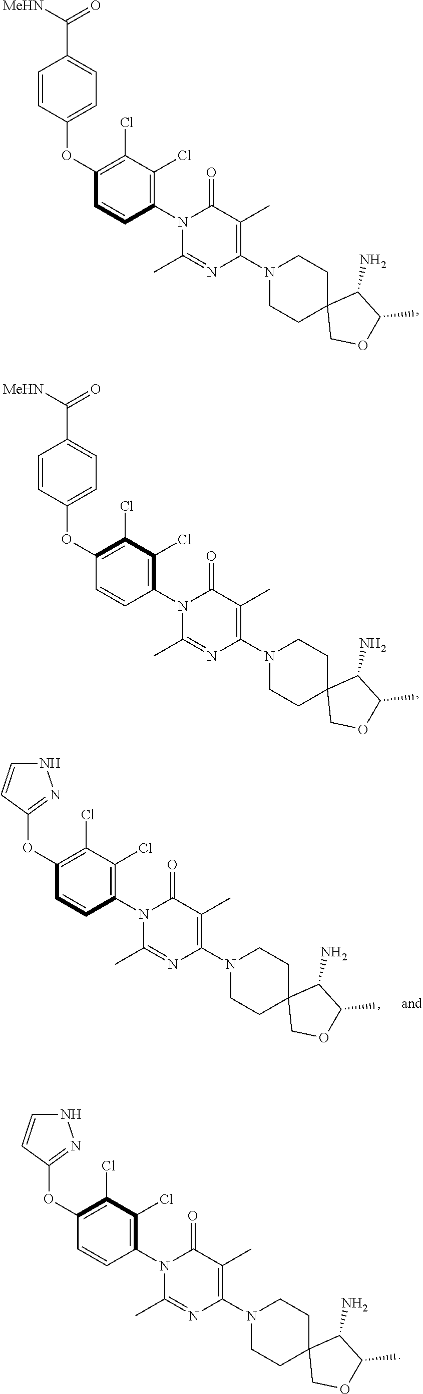

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound selected from the Examples disclosed herein.

### IV. Composition

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for parenteral administration.

In certain embodiments, the pharmaceutical composition is formulated for intravenous administration.

In certain embodiments, the pharmaceutical composition is formulated for subcutaneous administration.

In certain embodiments, the oral pharmaceutical composition is selected from a tablet and a capsule.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### V. Methods

The present invention also relates to a method of inhibiting at least one PTPN11 function comprising the step of contacting PTPN11 with a compound as described herein. The cell phenotype, cell proliferation, activity of PTPN11, change in biochemical output produced by active PTPN11, expression of PTPN11, or binding of PTPN11 with a natural binding partner may be monitored. Such methods may be modes of treatment of disease, biological assays, cellular assays, biochemical assays, or the like.

Also provided herein is a method of treatment of a PTPN11-mediated disease comprising the administration of a therapeutically effective amount of a compound as disclosed herein, or a salt or tautomer thereof, to a patient in need thereof.

In certain embodiments, the disease is cancer.

In certain embodiments, the cancer is selected from breast cancer, colon cancer, leukemia, or melanoma.

Also provided herein is a method of treatment of a PTP-mediated disease comprising the administration of a therapeutically effective amount of a compound as disclosed herein, or a salt or tautomer thereof, to a patient in need thereof.

In certain embodiments, the disease is selected from Noonan Syndrome and Leopard Syndrome.

In certain embodiments, the disease is cancer.

In certain embodiments, the cancer is selected from breast cancer, colon cancer, leukemia, or melanoma.

Also provided herein is a compound as disclosed herein for use as a medicament.

Also provided herein is a compound as disclosed herein for use as a medicament for the treatment of a PTPN11-mediated disease.

Also provided herein is a compound as disclosed herein for use as a medicament for the treatment of a PTP-mediated disease.

Also provided is the use of a compound as disclosed herein as a medicament.

Also provided is the use of a compound as disclosed herein as a medicament for the treatment of a PTPN11-mediated disease.

Also provided is a compound as disclosed herein for use in the manufacture of a medicament for the treatment of a PTPN11-mediated disease.

Also provided is the use of a compound as disclosed herein for the treatment of a PTPN11-mediated disease.

Also provided is the use of a compound as disclosed herein for the treatment of a PTP-mediated disease.

Also provided herein is a method of inhibition of PTPN11 comprising contacting PTPN11 with a compound as disclosed herein, or a salt or tautomer thereof.

Also provided herein is a method of inhibition of PTP comprising contacting PTP with a compound as disclosed herein, or a salt or tautomer thereof.

Also provided herein is a method for achieving an effect in a patient comprising the administration of a therapeutically effective amount of a compound as disclosed herein, or a salt or tautomer thereof, to a patient, wherein the effect is cognition enhancement.

In certain embodiments, the PTPN11-mediated disease is selected from Noonan Syndrome and Leopard Syndrome.

In certain embodiments, the PTPN11-mediated disease is cancer.

In certain embodiments, the PTPN11-mediated disease is selected from breast cancer, colon cancer, leukemia, or melanoma.

Also provided is a method of modulation of a PTPN11-mediated function in a subject comprising the administration of a therapeutically effective amount of a compound as disclosed herein.

### Administration and Combination Therapy

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, tautomer, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with anti-cancer (chemotherapeutic) drugs. Classes of anti-cancer drugs include, but are not limited to: alkylating agents, anti-metabolites, antimitotics, checkpoint inhibitors, plant alkaloids and terpenoids, topoisomerase inhibitors, cytotoxic antibiotics, aromatase inhibitors, angiogenesis inhibitors, anti-steroids and anti-androgens, mTOR inhibitors, tyrosine kinase inhibitors, and others.

For use in cancer and neoplastic diseases a PTPN11 (SHP2) inhibitor may be optimally used together with one or more of the following non-limiting examples of anti-cancer agents:
(1) alkylating agents, including but not limited to carmustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, and cyclophosphamide (ENDOXAN);
(2) anti-metabolites, including but not limited to cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEV), methotrexate (RHEUMATREX), raltitrexed;
(3) antimitotics, which are often plant alkaloids and terpenoids, or derivatives thereof, including but not limited to taxanes such as docetaxel (TAXITERE) and paclitaxel (ABRAXANE, TAXOL); vinca alkaloids such as vincristine (ONCOVIN), vinblastine, vindesine, and vinorelbine (NAVELBINE);
(4) checkpoint inhibitors, such as anti-PD-1 or PD-L1 antibodies pembrolizumab (KEYTRUDA), nivolumab (OPDIVO), MEDI4736, and MPDL3280A; anti-CTLA-4 antibody ipilimumab (YERVOY); and those that target LAG3 (lymphocyte activation gene 3 protein), KIR (killer cell immunoglobulin-like receptor), 4-1BB (tumour necrosis factor receptor superfamily member 9), TIM3 (T-cell immunoglobulin and mucin-domain containing-3) and OX40 (tumour necrosis factor receptor superfamily member 4);
(5) topoisomerase inhibitors, including but not limited to camptothecin (CTP), irinotecan (CAMPTOSAR), topotecan (HYCAMTIN), teniposide (VUMON), and etoposide (EPOSIN);
(6) cytotoxic antibiotics, including but not limited to actinomycin D (dactinomycin, COSMEGEN), bleomycin (BLENOXANE) doxorubicin (ADRIAMYCIN), daunorubicin (CERUBIDINE), epirubicin (ELLENCE), fludarabine (FLUDARA), idarubicin, mitomycin (MITOSOL), mitoxantrone (NOVANTRONE), plicamycin;
(7) aromatase inhibitors, including but not limited to aminoglutethimide, anastrozole (ARIMIDEX), letrozole (FEMARA), vorozole (RIVIZOR), exemestane (AROMASIN);
(8) angiogenesis inhibitors, including but not limited to genistein, sunitinib (SUTENT) and bevacizumab (AVASTIN);
(9) anti-steroids and anti-androgens such as aminoglutethimide (CYTADREN), bicalutamide (CASODEX), cyproterone, flutamide (EULEXIN), nilutamide (NILANDRON);
(10) tyrosine kinase inhibitors, including but not limited to imatinib (GLEEVEC), erlotinib (TARCEVA), lapatininb (TYKERB), sorafenib (NEXAVAR), and axitinib (INLYTA);
(11) mTOR inhibitors such as everolimus, temsirolimus (TORISEL), and sirolimus;
(12) monoclonal antibodies such as trastuzumab (HERCEPTIN) and rituximab (RITUXAN);
(13) other agents, such as amsacrine; Bacillus Calmette-Guerin (B-C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,p′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating PTPN11-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of PTPN11-mediated disorders.

In some embodiments, methods described herein are used to treat a disease condition comprising administering to a subject in need thereof a therapeutically effective amount of a compound of Formula I or pharmaceutically acceptable salt thereof, wherein the condition is cancer which has developed resistance to chemotherapeutic drugs and/or ionizing radiation.

In some embodiments, methods described herein are used to treat a disease condition comprising administering to a subject in need thereof a therapeutically effective amount of a compound of Formula I or pharmaceutically acceptable salt thereof, wherein the condition is cancer which has developed resistance to chemotherapeutic drugs and/or ionizing radiation.

The compounds, compositions, and methods disclosed herein are useful for the treatment of disease. In certain embodiments, the disease is one of dysregulated cellular proliferation, including cancer. The cancer may be hormone-dependent or hormone-resistant, such as in the case of breast cancers. In certain embodiments, the cancer is a solid tumor. In other embodiments, the cancer is a lymphoma or leukemia. In certain embodiments, the cancer is and a drug resistant phenotype of a cancer disclosed herein or known in the art. Tumor invasion, tumor growth, tumor metastasis, and angiogenesis may also be treated using the compositions and methods disclosed herein. Precancerous neoplasias are also treated using the compositions and methods disclosed herein.

Cancers to be treated by the methods disclosed herein include colon cancer, breast cancer, ovarian cancer, lung cancer and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML), and lymphomas including lymphocytic, granulocytic and monocytic. Additional types of cancers which may be treated using the compounds and methods of the invention include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumours, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor.

In certain embodiments, the compositions and methods disclosed herein are useful for preventing or reducing tumor invasion and tumor metastasis.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### VI. Methods of Preparation

The chirality of the molecule is described by specifying its configuration using the Cahn-Ingold-Prelog convention, using the descriptors R and S for central chirality and Rₐ and Sₐ for axial chirality. PAC, 1996, 68, 2193, Basic terminology of stereochemistry (IUPAC Recommendations 1996), doi:10.1351/pac199668122193; Preferred IUPAC Names Chapter 9, September, 2004. Cahn, C. K. Ingold and V. Prelog, Angew. Chem. Internat. Ed. Eng. 5, 385-415, 511 (1966); V. Prelog and G. Helmchen, Angew. Chem. Internat. Ed. Eng. 21, 567-583 (1982).

### Synthesis Schemes

The following schemes can be used to practice the present invention.

### General Synthetic Methods for Preparing Compounds

The following scheme can be used as general guidance to prepare compounds of the present invention.

List of Abbreviations: Pd₂dba₃—Tris(dibenzylideneacetone)dipalladium;

“\*MERGEFORMAT\*MERGEFORMAT Xantphos—4,5-Bis(diphenylphosphino)-9,9-dimethylxanthene; HCl=hydrogen chloride; TFA=trifluoroacetic acid; POCl₃—Phosphorus (V) oxychloride; Tf₂O—Trifluoromethanesulfonic anhydride; DIEA—Diisopropyl ethylamine; DCM=dichloromethane; BOP—(benzotriazol-1-yloxytris(dimethylamino)phosphonium hexafluorophosphate);

“\*MERGEFORMAT\*MERGEFORMAT DBU—1,8-Diazabicyclo[5.4.0]undec-7-ene; NBS—N-Bromoosuccinimide; and

“\*MERGEFORMAT\*MERGEFORMAT NCS—N-Chlorosuccinimide.

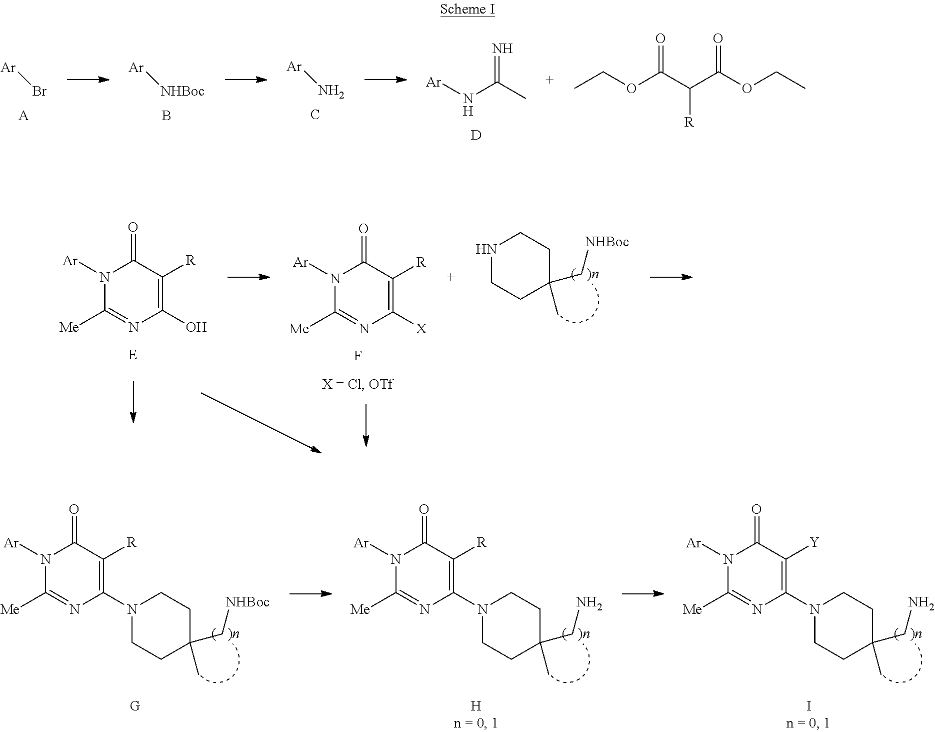

Aryl halide A can be transformed into the corresponding protected amine B using a “Pd” catalyst and ligand such as Pd₂dba₃ and Xantphos. Amine B can be deprotected to afford C and the corresponding acetimidamide D can be formed by treatment of amine C with acetonitrile and an acid such as HCl. Aryl substituted-6-hydroxy-2-methylpyrimidin-4(3H)-one E is formed by reacting D with the desired malonate to afford E. E can be transformed to the corresponding halide by treatment with reagent eg POCl₃ or triflate by Tf₂O and DIEA in DCM. Subsequent SNAr reaction can provide protected amine G that can be transformed to the unprotected amine H. F can directly transformed to H in an SNAr reaction. Direct conversion of E to H can be performed by applying reagents such as BOP and DBU. H can be further functionalized to the corresponding bromide, chloride or CF₃ derivative (I) by applying reagent such as NBS, NCS or bis(((trifluoromethyl)sulfinyl)oxy)zinc in the presence of tert-butyl hydroperoxide.

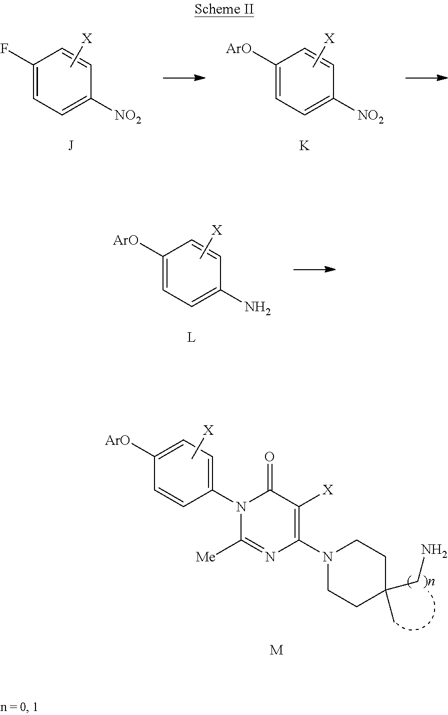

Fluoronitrobenzene derivative J can be transferred to corresponding ether K using an alcohol or phenol and a base such as sodium hydride under SNAr reaction conditions. The nitro derivative can be reduced to the amine L using a reducing agent such as SnCl₂·2H₂O. Compound L subsequently can be transformed to the desired pyrimidinone derivative M as described in Scheme I.

It will be appreciated that other synthetic routes may be available for practice of the present invention.

The invention is further illustrated by the following examples, which may be synthesized and isolated as free bases or as salts, for example TFA or HCl salts.

### VII. EXAMPLES

### List of Abbreviations

mg—milligram; mL—milliliter; ul=microliter; M=molar; mmol—millimol; h=hour; min.=minute; Rt—Room Temperature; N₂=nitrogen; HCl=hydrogen chloride; H₂O=water; MS=mass spectrometry; ES+=electrospray positive ionization; ¹H-NMR=proton nuclear magnetic resonance; MHz=megahertz; DMSO-d₆=dimethyl sulfoxide deuterated-6; H=hydrogen; rt=room temperature; ° C.=degrees Celsius; Br₂=bromine; NaOH=sodium hydroxide; NaHSO₃=sodium bisulfite; K₂CO₃=potassium carbonate; NMP=N-Methyl-2-pyrrolidone; BOP—(benzotriazol-1-yloxytris(dimethylamino)phosphonium hexafluorophosphate); DBU—1,8-Diazabicyclo[5.4.0]undec-7-ene; DIEA—Diisopropyl ethylamine; MW=microwave; KF=potassium fluoride; Pd(dppf)Cl₂=[1,1′-bis(diphenylphosphino)ferrocene]palladium(II) dichloride; Pd₂dba₃—Tris(dibenzylideneacetone)dipalladium; POCl₃—Phosphorus (V) oxychloride; PE=petroleum ether; EA=ethyl acetate; CDCl₃=deuterated chloroform; MeOH=methanol; D₂O=deuterated water; HPLC=high pressure liquid chromatography; DMSO=dimethyl sulfoxide; MeCN (or ACN)=acetonitrile; NIS=N-iodosuccinimide; DMF=dimethylformamide; K₃PO₄=potassium phosphate, tribasic; Et₂O—Diethyl ether; EtOAc—Ethyl acetate; EtOH—Ethanol; NaOMe—Sodium methoxide; NaOEt—Sodium ethoxide; NCS—N-Chlorosuccinimide; TBDMS=TBS=tert-butyldimethylsilyl; TFA=trifluoroacetic acid; DCM=dichloromethane; Xantphos—4,5-Bis(diphenylphosphino)-9,9-dimethylxanthene.

### Example 1

### 6-(4-Amino-4-methylpiperidin-1-yl)-3-(2,3-dichlorophenyl)-2-methylpyrimidin-4(3H)-one TFA Salt

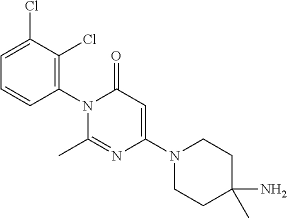 [SMILES: Cc1nc(N2CCC(C)(N)CC2)cc(=O)n1-c1cccc(Cl)c1Cl]

### Step 1: N-(2,3-Dichlorophenyl)acetimidamide

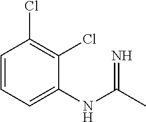

### Method A

To a mixture of 2,3-dichloroaniline (1.6 g, 10 mmol) and MeCN (615 mg, 15.0 mmol) in 1,2-dichloroethane (10 mL) was added AlCl₃ (1.46 g, 1.10 mmol) at 0° C. The reaction mixture was stirred for 10 min then it was heated to 100° C. for 18 h in a sealed tube. The reaction was cooled to rt, ice-water (30 mL) was added, and the aqueous phase was extracted with DCM (50 mL×2). Subsequently, 2M NaOH was added to adjust the pH to 10 and the aqueous phase was extracted with DCM (50 mL×3). The combined organic layers were dried over MgSO₄ and concentrated under reduced pressure to afford the title compound as a beige oil (950 mg, 47%). MS (ES+) C₈H₈Cl₂N₂ requires: 202, found: 203[M+H]⁺. The product was used in the next step without further purification.

### Method B

In a high pressure reaction vessel MeCN (123 mL) was bubbled with HCl gas for 15 seconds at 0° C. to give a saturated solution. 2,3-Dichloroaniline (7.30 ml, 61.7 mmol) was added and a white solid precipitate formed. The vessel was sealed and the mixture was heated at 120° C. for 16 h. The reaction mixture was cooled to rt and the white precipitate was collected by vacuum filtration. The solid was rinsed with Et₂O (20 mL×3) and dried under vacuum to afford the title compound (14.5 g, 98% yield) as a white solid. MS (ES+) C₈H₈Cl₂N₂ requires: 202, found: 203 [M+H]⁺.

### Step 2: 3-(2,3-Dichlorophenyl)-6-hydroxy-2-methylpyrimidin-4(3H)-one

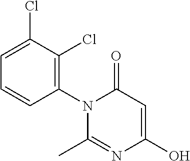 [SMILES: Cc1nc(O)cc(=O)n1-c1cccc(Cl)c1Cl]

### Method A

To a solution of N-(2,3-dichlorophenyl)acetimidamide (950 mg, 4.68 mmol) in 2-methoxyethan-1-ol (10 mL) was added diethyl malonate (2.99 g, 18.7 mmol) and NaOMe in MeOH (4M, 4.7 mL, 18 mmol). The mixture was heated at 120° C. for 16 h. The reaction mixture was cooled to rt, it was poured onto water (50 mL) and it was extracted with Et₂O (50 mL). The aqueous layer was acidified to pH-2 with HCl (6M, aq.) and the solid precipitate was collected by filtration to afford the title compound (1 g, 77%) as a white solid. MS (ES+) C₁₁H₈Cl₂N₂O₂ requires: 270, found: 271[M+H]⁺.

### Method B

To a suspension of N-(2,3-dichlorophenyl)acetimidamide (30 g, 125 mmol) in EtOH (125 mL) were added diethyl malonate (38.2 mL, 250 mmol) and NaOEt (20% in EtOH) (140 mL, 376 mmol) and the resulting mixture was stirred in a sealed tube at 120° C. for 18 h. The reaction mixture was allowed to cool to room temperature and the volatiles were removed under reduced pressure. Water (30 mL) was added to the residue, the mixture was cooled to 0° C. and the mixture was acidified to pH-2 with HCl (6M, aq). The mixture was allowed to warm to rt and stirred for 1 h. The solid was collected by vacuum filtration, it was rinsed with Et₂O (20 mL×2) and dried under reduced pressure to afford the title compound (26.3 g, 77%) as a tan solid (mixture of enantiomers). MS (ES+) C₁₁H₈C₂N₂O₂ requires: 270, found: 271 [M+H]⁺.

The compound of Step 2 can exist as a mixture of 3-(Sₐ)-(2,3-Dichlorophenyl)-6-hydroxy-2-methylpyrimidin-4(3H)-one and 3-(Rₐ)-(2,3-dichlorophenyl)-6-hydroxy-2-methylpyrimidin-4(3H)-one

### Step 3: 6-Chloro-3-(2,3-dichlorophenyl)-2-methylpyrimidin-4(3H)-one

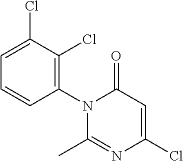 [SMILES: Cc1nc(Cl)cc(=O)n1-c1cccc(Cl)c1Cl]

A mixture of 1-(2,3-dichlorophenyl)-2-methylpyrimidine-4,6(1H,5H)-dione (250 mg, 0.92 mmol) in POCl₃ (5 mL) was heated at 100° C. for 4 hrs. The reaction mixture was cooled to rt, it was concentrated under reduced pressure and it was slowly added to ice-water. The mixture was extracted with EtOAc (15 mL×3). The organic layer was dried and concentrated under reduced pressure. The residue was purified by flash column chromatography (EtOAc in PE, 20 to 50%) to afford the title compound as a beige solid (100 mg, 37.5%) (mixture of enantiomers). MS (ES+) C₁₁H₇Cl₃N₂O requires: 288, found: 289[M+H]⁺.

The compound of Step 3 can exist as a mixture of 6-Chloro-3-(Sₐ)-(2,3-dichlorophenyl)-2-methylpyrimidin-4(3H)-one and 6-chloro-3-(Rₐ)-(2,3-dichlorophenyl)-2-methylpyrimidin-4(3H)-one.

### Step 4: Tert-butyl (1-(1-(2,3-dichlorophenyl)-2-methyl-6-oxo-1,6-dihydropyrimidin-4-yl)-4-methylpiperidin-4-yl)carbamate

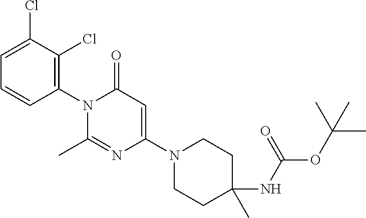 [SMILES: Cc1nc(N2CCC(C)(NC(=O)OC(C)(C)C)CC2)cc(=O)n1-c1cccc(Cl)c1Cl]

A mixture of 6-chloro-3-(2,3-dichlorophenyl)-2-methylpyrimidin-4(3H)-one (100 mg, 0.35 mmol), tert-butyl (4-methylpiperidin-4-yl)carbamate (89 mg, 0.42 mmol) and DIEA (134 mg, 1.04 mmol) in DMF (3 mL) was stirred at 100° C. for 3 h. The reaction mixture was diluted with water, it was extracted with EtOAc (15 mL×3). The organic layer was washed with brine, dried over MgSO₄ and concentrated under reduced pressure to afford the title compound. MS (ES+) C₂₂H₂₈Cl₂N₄O₃ requires: 466, found: 467 [M+H]⁺. The product was used in the next step without further purification.

The compound of Step 4 can exist as a mixture of Tert-butyl (1-(1-(Sₐ)-(2,3-dichlorophenyl)-2-methyl-6-oxo-1,6-dihydropyrimidin-4-yl)-4-methylpiperidin-4-yl)carbamate and tert-butyl (1-(1-(Rₐ)-(2,3-dichlorophenyl)-2-methyl-6-oxo-1,6-dihydropyrimidin-4-yl)-4-methylpiperidin-4-yl)carbamate.

### Step 5: 6-(4-amino-4-methylpiperidin-1-yl)-3-(2,3-dichlorophenyl)-2-methylpyrimidin-4(3H)-one TFA Salt

To a solution of tert-butyl (1-(1-(2,3-dichlorophenyl)-2-methyl-6-oxo-1,6-dihydropyrimidin-4-yl)-4-methylpiperidin-4-yl)carbamate (0.35 mmol, crude) in DCM (3 mL) was added TFA (1 mL). The resulting solution was stirred at room temperature for 2 h and concentrated under reduced pressure. The residue was purified by prep-HPLC (Mobile phase: A=0.01% TFA/H2O, B=MeCN; Gradient: B=60%-95% in 18 min; Column: Venusil CBP C18 (L) C18, 10 μm, 21.2 mm×250 mm, Cat. NO.: VX902520-L) to afford the title compound as a white solid (110 mg, 86%); MS (ES+) C₁₇H₂₀Cl₂N₄O requires: 366, found: 367 [M+H]⁺; 1H NMR (500 MHz, DMSO) δ 8.03 (s, 3H), 7.80 (dd, J=7.8, 1.7 Hz, 1H), 7.61-7.46 (m, 2H), 5.47 (s, 1H), 4.08-3.82 (m, 2H), 3.34-3.29 (m, 2H), 2.00 (s, 3H), 1.71 (t, J=5.5 Hz, 4H), 1.37 (s, 3H).

The compound of Example 1 can exist as a mixture of 6-(4-amino-4-methylpiperidin-1-yl)-3-(Sₐ)-(2,3-dichlorophenyl)-2-methylpyrimidin-4(3H)-one TFA salt and 6-(4-amino-4-methylpiperidin-1-yl)-3-(Rₐ)-(2,3-dichlorophenyl)-2-methylpyrimidin-4(3H)-one TFA salt.

### Example 2a and 2b

### 6-((3S,4S)-4-Amino-3-methyl-2-oxa-8-azaspiro[4.5]decan-8-yl)-3-(Sₐ)-(2,3-dichlorophenyl)-2-methylpyrimidin-4(3H)-one (Example 2a

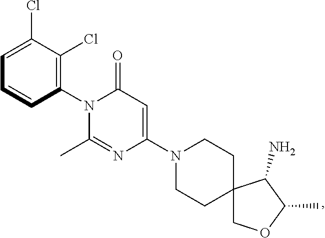

### 6-((3S,4S)-4-amino-3-methyl-2-oxa-8-azaspiro[4.5]decan-8-yl)-3-(Rₐ)-(2,3-dichlorophenyl)-2-methylpyrimidin-4(3H)-one (Example 2b

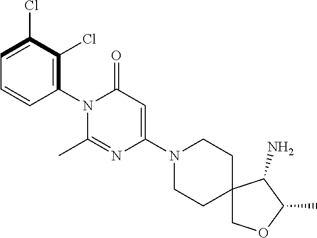

To a suspension of 3-(2,3-dichlorophenyl)-6-hydroxy-2-methylpyrimidin-4(3H)-one (Example 1, Step 2, 3.50 g, 12.9 mmol) and (3S,4S)-3-methyl-2-oxa-8-azaspiro[4.5]decan-4-amine dihydrochloride (3.45 g, 14.2 mmol) in MeCN (43 ml) were added DBU (6.23 mL, 41.3 mmol) and BOP (6.85 g, 15.5 mmol) and the resulting mixture was stirred at rt for 24 h. The mixture was concentrated under reduced pressure. The residue was purified via silica gel chromatography (2-15% MeOH with 2% NH₄OH in DCM) to afford the title compounds as a mixture of diastereomers (4.2 g, 77% yield) as an off-white solid. The mixture of diastereomers were separated by Chiral SFC (Mobile phase: CO₂/methanol (0.25% isopropylamine)=60/40; Flow rate: 80 g/min; 5 min; Column temperature: 25° C.; Back pressure: 100 bar; Column: ES Industries ChromegaChiral CCS, 2.0×25.0 cm) to afford the title compounds, Example 2a (19.7 g, 48%) as a white solid and Example 2b (11.6 g, 28%) as a white solid.

Example 2a 6-((3S,4S)-4-Amino-3-methyl-2-oxa-8-azaspiro[4.5]decan-8-yl)-3-(Sₐ)-(2,3-dichlorophenyl)-2-methylpyrimidin-4(3H)-one: MS (ES+) C₂₀H₂₄Cl₂N₄O₂ requires: 422, found: 423 [M+H]⁺; ¹H NMR (600 MHz, CDCl₃) δ 7.57 (dd, J=8.1, 1.5 Hz, 1H), 7.35 (t, J=8.0 Hz, 1H), 7.21 (dd, J=7.9, 1.5 Hz, 1H), 5.45 (s, 1H), 4.21-4.16 (m, 1H), 3.86 (br s, 2H), 3.79 (d, J=8.7 Hz, 1H), 3.68 (d, J=8.7 Hz, 1H), 3.40 (ddd, J=13.1, 9.1, 3.8 Hz, 1H), 3.31 (ddd, J=13.3, 9.4, 3.2 Hz, 1H), 2.98 (d, J=4.5 Hz, 1H), 2.06 (s, 3H), 1.84 (ddd, J=13.4, 9.4, 3.9 Hz, 1H), 1.75-1.60 (m, 3H), 1.23 (d, J=6.4 Hz, 3H).

Example 2b 6-((3S,4S)-4-Amino-3-methyl-2-oxa-8-azaspiro[4.5]decan-8-yl)-3-(Rₐ)-(2,3-dichlorophenyl)-2-methylpyrimidin-4(3H)-one: MS (ES+) C₂₀H₂₄Cl₂N₄O₂ requires: 422, found: 423 [M+H]⁺; ¹H NMR (600 MHz, CDCl₃) δ 7.57 (dd, J=8.2, 1.5 Hz, 1H), 7.36 (t, J=8.0 Hz, 1H), 7.21 (dd, J=7.9, 1.5 Hz, 1H), 5.46 (s, 1H), 4.22-4.14 (m, 1H), 3.95-3.82 (m, 2H), 3.80 (d, J=8.7 Hz, 1H), 3.68 (d, J=8.7 Hz, 1H), 3.45-3.36 (m, 1H), 3.29 (ddd, J=13.3, 9.5, 3.3 Hz, 1H), 2.99 (d, J=4.6 Hz, 1H), 2.07 (s, 3H), 1.84 (ddd, J=13.4, 9.4, 3.9 Hz, 1H), 1.77-1.70 (m, 1H), 1.70-1.61 (m, 2H), 1.24 (d, J=6.4 Hz, 3H).

The following Examples were synthesized with synthetic methods that were similar to that used for Example 1 and 2, and can generally be made by methods disclosed herein. The Examples may be made as free bases or as TFA salts.

TABLE 1
Examples 3-8

| | | Calc. | Mass |
|---|---|---|---|
| Ex # | Structure and IUPAC Name | Mass | [M + H]⁺ |
| 3 | 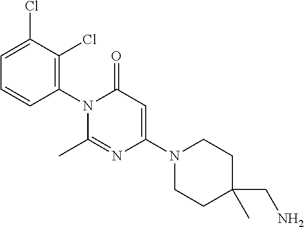 [SMILES: Cc1nc(N2CCC(C)(CN)CC2)cc(=O)n1-c1cccc(Cl)c1Cl] | 380 | 381 |
| | 6-[4-(aminomethyl)-4-methylpiperidin-1-yl]-3- | | |
| | (2,3-dichlorophenyl)-2-methyl-3,4- | | |
| | dihydropyrimidin-4-one | | |
| 4 | 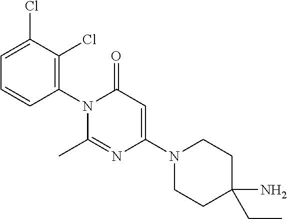 [SMILES: CCC1(N)CCN(c2cc(=O)n(-c3cccc(Cl)c3Cl)c(C)n2)CC1] | 380 | 381 |
| | 6-(4-amino-4-ethylpiperidin-1-yl)-3-(2,3- | | |
| | dichlorophenyl)-2-methyl-3,4- | | |
| | dihydropyrimidin-4-one | | |
| 5 | 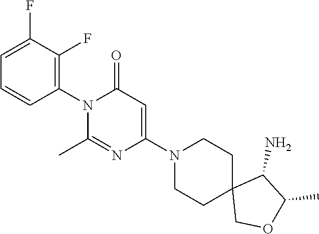 [SMILES: Cc1nc(N2CCC3(CC2)CO[C@@H](C)[C@H]3N)cc(=O)n1-c1cccc(F)c1F] | 390 | 391 |
| | 6-[(3S,4S)-4-amino-3-methyl-2-oxa-8- | | |
| | azaspiro[4.5]decan-8-yl]-3-(2,3-difluorophenyl)-2- | | |
| | methyl-3,4-dihydropyrimidin-4-one | | |
| 6 | 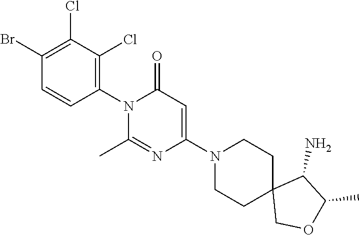 [SMILES: Cc1nc(N2CCC3(CC2)CO[C@@H](C)[C@H]3N)cc(=O)n1-c1ccc(Br)c(Cl)c1Cl] | 500/502 | 501/503 |
| | 6-((3S,4S)-4-amino-3-methyl-2-oxa-8- | | |
| | azaspiro[4.5]decan-8-yl)-3-(4-bromo-2,3- | | |
| | dichlorophenyl)-2-methylpyrimidin-4(3H)-one | | |
| 7a | 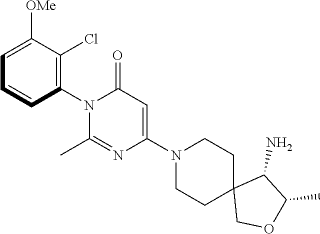 | 418 | 419 |
| | 6-((3S,4S)-4-amino-3-methyl-2-oxa-8- | | |
| | azaspiro[4.5]decan-8-yl)-3-(Sₐ)-(2-chloro-3- | | |
| | methoxyphenyl)-2-methylpyrimidin-4(3H)-one | | |
| 7b | 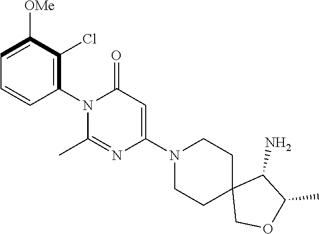 | 418 | 419 |
| | 6-((3S,4S)-4-amino-3-methyl-2-oxa-8- | | |
| | azaspiro[4.5]decan-8-yl)-3-(Rₐ)-(2-chloro-3- | | |
| | methoxyphenyl)-2-methylpyrimidin-4(3H)-one | | |
| 8 | 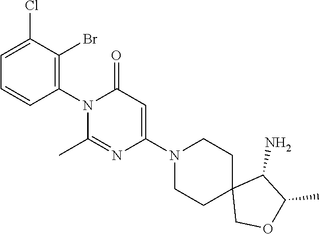 [SMILES: Cc1nc(N2CCC3(CC2)CO[C@@H](C)[C@H]3N)cc(=O)n1-c1cccc(Cl)c1Br] | 466 | 467 |
| | 6-((3S,4S)-4-amino-3-methyl-2-oxa-8- | | |
| | azaspiro[4.5]decan-8-yl)-3-(2-bromo-3- | | |
| | chlorophenyl)-2-methylpyrimidin-4(3H)-one | | |

The compound of Example 3 can exist as a mixture of 6-[4-(aminomethyl)-4-methylpiperidin-1-yl]-3-(Sₐ)-(2,3-dichlorophenyl)-2-methyl-3,4-dihydropyrimidin-4-one and 6-[4-(aminomethyl)-4-methylpiperidin-1-yl]-3-(Rₐ)-(2,3-dichlorophenyl)-2-methyl-3,4-dihydropyrimidin-4-one.

The compound of Example 4 can exist as a mixture of 6-(4-amino-4-ethylpiperidin-1-yl)-3-(Sₐ)-(2,3-dichlorophenyl)-2-methyl-3,4-dihydropyrimidin-4-one and 6-(4-amino-4-ethylpiperidin-1-yl)-3-(Rₐ)-(2,3-dichlorophenyl)-2-methyl-3,4-dihydropyrimidin-4-one.

The compound of Example 5 can exist as a mixture of 6-[(3S,4S)-4-amino-3-methyl-2-oxa-8-azaspiro[4.5]decan-8-yl]-3-(Sₐ)-(2,3-difluorophenyl)-2-methyl-3,4-dihydropyrimidin-4-one and 6-[(3S,4S)-4-amino-3-methyl-2-oxa-8-azaspiro[4.5]decan-8-yl]-3-(Rₐ)-(2,3-difluorophenyl)-2-methyl-3,4-dihydropyrimidin-4-one.

The compound of Example 6 can exist as a mixture of 6-((3S,4S)-4-amino-3-methyl-2-oxa-8-azaspiro[4.5]decan-8-yl)-3-(Sₐ)-(4-bromo-2,3-dichlorophenyl)-2-methylpyrimidin-4(3H)-one and 6-((3S,4S)-4-amino-3-methyl-2-oxa-8-azaspiro[4.5]decan-8-yl)-3-(Rₐ)-(4-bromo-2,3-dichlorophenyl)-2-methylpyrimidin-4(3H)-one.

The compound of Example 8 can exist as a mixture of 6-((3S,4S)-4-amino-3-methyl-2-oxa-8-azaspiro[4.5]decan-8-yl)-3-(Sₐ)-(2-bromo-3-chlorophenyl)-2-methylpyrimidin-4(3H)-one and 6-((3S,4S)-4-amino-3-methyl-2-oxa-8-azaspiro[4.5]decan-8-yl)-3-(Rₐ)-(2-bromo-3-chlorophenyl)-2-methylpyrimidin-4(3H)-one.

### Example 9

### 6-((3S,4S)-4-Amino-3-methyl-2-oxa-8-azaspiro[4.5]decan-8-yl)-5-chloro-3-(Rₐ)-(2,3-dichlorophenyl)-2-methylpyrimidin-4(3H)-one

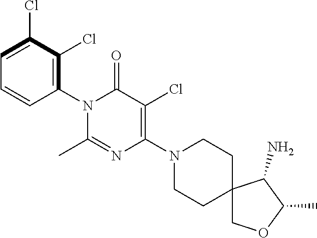

To a solution of 6-((3S,4S)-4-amino-3-methyl-2-oxa-8-azaspiro[4.5]decan-8-yl)-3-(Rₐ)-(2,3-dichlorophenyl)-2-methylpyrimidin-4(3H)-one (Example 2b; 2.5 g, 5.9 mmol) in DCM (30 mL) was added NCS (946 mg, 7.09 mmol) and the resulting mixture was stirred at rt for 10 min. The mixture was concentrated under reduced pressure. The residue was purified via silica gel chromatography (2-15% MeOH with 2% NH₄OH in DCM) to afford the title compound (2.1 g, 78% yield) as a white solid. MS (ES+) C₂₀H₂₃Cl₃N₄O₂ requires: 456, found: 457 [M+H]⁺;

“\*MERGEFORMAT\*MERGEFORMAT ¹H NMR (600 MHz, DMSO-d₆) δ 7.83 (dd, J=8.1, 1.6 Hz, 1H), 7.62 (dd, J=8.0, 1.6 Hz, 1H), 7.57 (t, J=8.0 Hz, 1H), 4.09-4.01 (m, 1H), 3.96-3.87 (m, 2H), 3.67 (d, J=8.4 Hz, 1H), 3.53-3.46 (m, 2H), 3.40 (ddd, J=12.9, 9.3, 3.1 Hz, 1H), 2.91 (d, J=5.1 Hz, 1H), 2.00 (s, 3H), 1.81 (ddd, J=13.2, 9.3, 3.6 Hz, 1H), 1.71-1.65 (m, 1H), 1.59-1.54 (m, 1H), 1.54-1.48 (m, 1H), 1.08 (d, J=6.4 Hz, 3H).

### Example 10a and 10b

### 6-((3S,4S)-4-Amino-3-methyl-2-oxa-8-azaspiro[4.5]decan-8-yl)-3-(Sₐ)-(2,3-dichlorophenyl)-2,5-dimethylpyrimidin-4(3H)-one (Example 10a

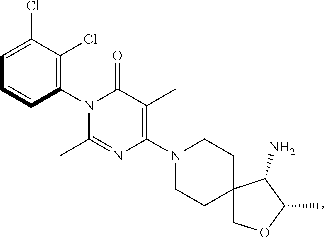

and

### 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one (Example 10b

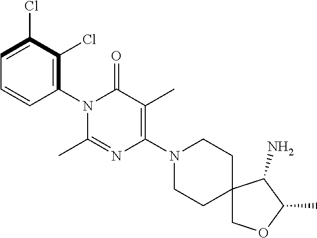

### Method A

### Step 1: 3-(2,3-Dichlorophenyl)-6-hydroxy-2,5-dimethylpyrimidin-4(3H)-one

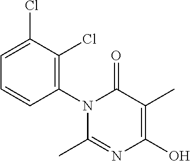 [SMILES: Cc1c(O)nc(C)n(-c2cccc(Cl)c2Cl)c1=O]

To a suspension of N-(2,3-dichlorophenyl)acetimidamide hydrochloride (Example 1, step 1) (44 g, 184 mmol) in EtOH (184 mL) were added diethyl 2-methylmalonate (62.6 ml, 367 mmol) and EtONa (206 ml, 551 mmol) and the resulting mixture was stirred in a sealed tube at 120° C. for 18 h. The reaction mixture was allowed to cool to room temperature and the volatiles were removed under reduced pressure. Water (200 mL) was added, the mixture was cooled to 0° C., and HCl (6 M) was added to adjust the pH to 2. The reaction was allowed to warm to rt and was stirred for 1 h. The reaction mixture was filtered through glass fiber paper, the solid was rinsed with Et₂O, collected, and dried under reduced pressure to afford the title compound (44.6 g, 156 mmol, 85% yield) as a pale yellow solid. MS (ES+) C₁₂H₁₀C₁₂N₂O₂ requires: 284, found: 285 [M+H]⁺.

The compound of Step 1 can exist as a mixture of 3-(Sₐ)-(2,3-dichlorophenyl)-6-hydroxy-2,5-dimethylpyrimidin-4(3H)-one and 3-(Rₐ)-(2,3-dichlorophenyl)-6-hydroxy-2,5-dimethylpyrimidin-4(3H)-one.

### Step 2: 6-((3S,4S)-4-Amino-3-methyl-2-oxa-8-azaspiro[4.5]decan-8-yl)-3-(Sₐ)-(2,3-dichlorophenyl)-2,5-dimethylpyrimidin-4(3H)-one (Example 10a) and 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one (Example 10b

To a suspension of 3-(2,3-dichlorophenyl)-6-hydroxy-2,5-dimethylpyrimidin-4(3H)-one (30 g, 105 mmol) and (3S,4S)-3-methyl-2-oxa-8-azaspiro[4.5]decan-4-amine dihydrochloride (28.1 g, 116 mmol) in acetonitrile (351 mL) were added DBU (50.8 mL, 337 mmol) and BOP (51.2 g, 116 mmol) and the resulting mixture was stirred at rt for 24 h. (3S,4S)-3-Methyl-2-oxa-8-azaspiro[4.5]decan-4-amine dihydrochloride (12.8 g, 52.5 mmol) and DBU (15.8 mL, 105 mmol) were added and the mixture was stirred at rt for an additional 24 h. The reaction mixture was concentrated under reduced pressure. The residue was partitioned between DCM (200 mL) and water (200 mL), the layers were separated and the aqueous layer was extracted with DCM (200 mL×3). The combined organic layers were dried over Na₂SO₄, filtered and concentrated under reduced pressure. The residue was purified via silica gel chromatography (2-15% MeOH with 2% NH₄OH in DCM) to afford the title compounds as a mixture of diastereomers as an off-white solid. The mixture of diastereomers were separated by Chiral SFC (Mobile phase: CO₂/methanol (0.25% isopropylamine)=60/40; Flow rate: 80 g/min; 5 min; Column temperature: 25° C.; Back pressure: 100 bar; Column: Chiral Technologies Chiralpak AD-H, 2.1×25.0 cm) to afford the title compounds Example 10a (19.51 g, 42%, 98% ee) as a white solid (a single diastereomer) and Example 10b (19.25 g, 42%, >99% ee) as a white solid (a single diastereomer).

Example 10a 6-((3S,4S)-4-Amino-3-methyl-2-oxa-8-azaspiro[4.5]decan-8-yl)-3-(Sₐ)-(2,3-dichlorophenyl)-2,5-dimethylpyrimidin-4(3H)-one: MS (ES+) C₂₁H₂₆Cl₂N₄O₂ requires: 436, found: 437 [M+H]⁺; ¹H NMR (600 MHz, DMSO-d₆) δ 7.83-7.77 (m, 1H), 7.57-7.50 (m, 2H), 4.09-4.02 (m, 1H), 3.66 (d, J=8.4 Hz, 1H), 3.58-3.51 (m, 2H), 3.49 (d, J=8.4 Hz, 1H), 3.21 (ddd, J=12.9, 9.3, 3.2 Hz, 1H), 3.13 (ddd, J=12.9, 9.4, 3.0 Hz, 1H), 2.89 (d, J=5.1 Hz, 1H), 1.97 (s, 3H), 1.89 (s, 3H), 1.78 (ddd, J=13.3, 9.5, 3.6 Hz, 1H), 1.66 (ddd, J=13.1, 9.1, 3.7 Hz, 1H), 1.57-1.52 (m, 1H), 1.52-1.47 (m, 1H), 1.08 (d, J=6.4 Hz, 3H).

Example 10b 6-((3S,4S)-4-Amino-3-methyl-2-oxa-8-azaspiro[4.5]decan-8-yl)-3-(Rₐ)-(2,3-dichlorophenyl)-2,5-dimethylpyrimidin-4(3H)-one: MS (ES+) C₂₁H₂₆Cl₂N₄O₂ requires: 436, found: 437 [M+H]⁺; ¹H NMR (600 MHz, DMSO-d₆) δ 7.83-7.77 (t, J=4.8 Hz, 1H), 7.57-7.50 (d, J=4.8 Hz, 2H), 4.09-4.02 (m, 1H), 3.65 (d, J=8.4 Hz, 1H), 3.58-3.51 (m, 2H), 3.49 (d, J=8.4 Hz, 1H), 3.22 (ddd, J=13, 9.5, 2.5 Hz, 1H), 3.12 (ddd, J=13.0, 9.5, 2.5 Hz, 1H), 2.89 (d, J=5.1 Hz, 1H), 1.97 (s, 3H), 1.89 (s, 3H), 1.78 (ddd, J=13.2, 9.5, 3.7 Hz, 1H), 1.66 (ddd, J=13.2, 9.5, 3.7 Hz, 1H), 1.58-1.46 (m, 2H), 1.28 (bs, 2H), 1.08 (d, J=6.4 Hz, 3H).

### Method B

### Step 1: 1-(Rₐ)-(2,3-Dichlorophenyl)-2,5-dimethyl-6-oxo-1,6-dihydropyrimidin-4-yl trifluoromethanesulfonate

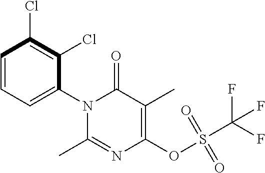

To a mixture of 3-(Sₐ)-(2,3-dichlorophenyl)-6-hydroxy-2,5-dimethylpyrimidin-4(3H)-one and 3-(Rₐ)-(2,3-dichlorophenyl)-6-hydroxy-2,5-dimethylpyrimidin-4(3H)-one (Example 10, Method A, Step 1) (100 g, 351 mmol) and DIPEA (183 mL, 1.05 mol,) in DCM (1.00 L) was added Tf₂O (116 mL g, 701 mmol, 116 mL) drop-wise at −25° C. The resulting mixture was stirred at −25° C.˜−20° C. for 1 h and at 20° C. for 18 h. Two batches of the reaction mixture were combined. To the reaction mixture was added water (1 L), the resulting mixture was extracted with CH₂Cl₂ (1 L×2). The combined organic layers were washed with 1M HCl (1 L, aq), dried over Na₂SO₄, filtered and concentrated under reduced pressure. The residue was purified by column chromatography (EtOAc in PE, 20%) to afford a mixture of diastereomers (144 g, 336 mmol, 47.8% yield) as a brown solid. The mixture of diastereomers was separated by SFC (column: DAICEL CHIRALPAK AD (250 mm*50 mm, 10 um); mobile phase: [Neu-IPA]; B %: 15%-15%, 2.9 min, 1100 min) to afford the title compound (56.4 g, 18%, ee=99.5%) as a brown solid. MS (ES+) C₁₃H₉Cl₂F₃N₂O₄S requires: 416, found: 417 [M+H]⁺.

### Step 2: 6-((3S,4S)-4-Amino-3-methyl-2-oxa-8-azaspiro[4.5]decan-8-yl)-3-(Rₐ)-(2,3-dichlorophenyl)-2,5-dimethylpyrimidin-4(3H)-one (Example 10b

To a solution of (3S,4S)-3-methyl-2-oxa-8-azaspiro[4.5]decan-4-amine (23.3 g, 95.6 mmol, 1.05 eq, 2HCl) in DMF (50.0 mL) was added DIPEA (47.1 g, 364 mmol, 63.5 mL) and a solution of 1-(Rₐ)-(2,3-dichlorophenyl)-2,5-dimethyl-6-oxo-1,6-dihydropyrimidin-4-yl trifluoromethanesulfonate (38.0 g, 91.1 mmol, 1.00 eq) in DMF (200 mL) at 0° C. The resulting mixture was stirred at 15° C. for 18 h and (3S,4S)-3-methyl-2-oxa-8-azaspiro[4.5]decan-4-amine (1.11 g, 4.55 mmol, 2HCl) was added. The resulting mixture was stirred at 25° C. for 15 h. The reaction mixture was concentrated under reduced pressure. K₂CO₃ (15%, 500 mL, aq.) was added and the aqueous phase was extracted with DCM (200 mL×3). The combined organic layers were washed with brine (400 mL), dried over Na₂SO₄, filtered and concentrated under reduced pressure. The residue was purified by column chromatography (SiO₂, MeOH in DCM, 5 to 20%) followed by reversed-phase column chromatography (0.1% NH₃·H₂O, MeCN:H2O (65:35). The residue was dissolved in hot MeOH (250 mL), to the resulting solution was added activated carbon (2.70 g) and the mixture was stirred at 60° C. for 15 min. The mixture was filtered and concentrated under reduced pressure to afford the title compound Example 10b (26.5 g, 66.5%) as off-white solid.

### Example 11

### 6-((3S,4S)-4-Amino-3-methyl-2-oxa-8-azaspiro[4.5]decan-8-yl)-3-(Rₐ)-(2,3-dichlorophenyl)-2-methyl-5-(trifluoromethyl)pyrimidin-4(3H)-one TFA salt

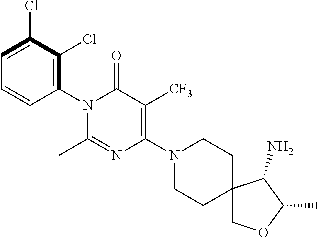

A suspension of 6-((3S,4S)-4-amino-3-methyl-2-oxa-8-azaspiro[4.5]decan-8-yl)-5-chloro-3-(Rₐ)-(2,3-dichlorophenyl)-2-methylpyrimidin-4(3H)-one (Example 9) (35 mg, 0.083 mmol), TFA (9.6 μL, 0.12 mmol) and bis(((trifluoromethyl)sulfinyl)oxy)zinc (55 mg, 0.16 mmol) in DCE (591 μL) was sonicated to give a uniform suspension then cooled to ° C. and tert-butyl hydroperoxide (34 μL, 0.25 mmol) was added dropwise. The resulting mixture was warmed to rt and stirred for 2 h. Water (1 mL) was added, the layers were separated and the aqueous layer was extracted with DCM (1 mL×3). The combined organic layers were dried over Na₂SO₄, filtered and concentrated under reduced pressure. The residue was purified by mass-triggered preparative HPLC (Mobile phase: A=0.1% TFA/H₂O, B=0.1% TFA/MeCN; Gradient: B=20-50%; 20 min; Column: XBridge C18, 5 μm, 19 mm×150 mm) to afford the title compound (15 mg, 30% yield) as a white solid (single diastereomer): MS (ES+) C₂₁H₂₃Cl₂F₃N₄O₂ requires: 490, found: 491 [M+H]⁺; ¹H NMR (600 MHz, DMSO-d₆) δ 7.90 (s, 3H), 7.84 (dd, J=7.9, 1.8 Hz, 1H), 7.63-7.56 (m, 2H), 4.24-4.17 (m, 1H), 3.96 (d, J=14.0 Hz, 1H), 3.91-3.83 (m, 2H), 3.72 (d, J=9.0 Hz, 1H), 3.50-3.44 (m, overlap H₂O, 1H), 3.32-3.16 (m, overlap H₂O, 2H), 2.03 (s, 3H), 1.78 (d, J=13.8 Hz, 3H), 1.61 (d, J=13.2 Hz, 1H), 1.21 (d, J=6.5 Hz, 3H).

### Example 12a and 12b

### 6-((3S,4S)-4-Amino-3-methyl-2-oxa-8-azaspiro[4.5]decan-8-yl)-5-bromo-3-(Sₐ)-(2,3-dichlorophenyl)-2-methylpyrimidin-4(3H)-one (Example 12a

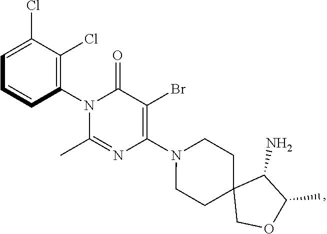

and

### 6-((3S,4S)-4-amino-3-methyl-2-oxa-8-azaspiro[4.5]decan-8-yl)-5-bromo-3-(Rₐ)-(2,3-dichlorophenyl)-2-methylpyrimidin-4(3H)-one (Example 12b

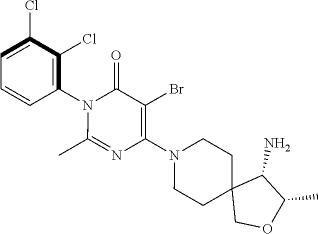

### Step 1: 5-Bromo-(2,3-dichlorophenyl)-6-hydroxy-2-methylpyrimidin-4(3H)-one

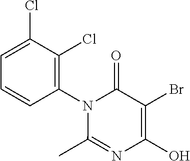 [SMILES: Cc1nc(O)c(Br)c(=O)n1-c1cccc(Cl)c1Cl]

To a solution of 3-(2,3-dichlorophenyl)-6-hydroxy-2-methylpyrimidin-4(3H)-one (Example 1, step 2) (540 mg, 2 mmol) in DCM (8 mL) was added a solution of NBS (356 mg, 2 mmol) in DCM (2 mL). The reaction mixture was stirred at rt for 2 h. The reaction mixture was filtered to afford the title compound as a white solid (680 mg, 97.1%). MS (ES+) C₁₁H₇BrCl₂N₂O₂ requires: 350, found: 351[M+H]⁺.

The compound of Step 1 can exist as a mixture of 5-bromo-(Sₐ)-(2,3-dichlorophenyl)-6-hydroxy-2-methylpyrimidin-4(3H)-one and 5-bromo-(Rₐ)-(2,3-dichlorophenyl)-6-hydroxy-2-methylpyrimidin-4(3H)-one.

### Step 2: 6-((3S,4S)-4-Amino-3-methyl-2-oxa-8-azaspiro[4.5]decan-8-yl)-5-bromo-3-(Rₐ)-(2,3-dichlorophenyl)-2-methylpyrimidin-4(3H)-one (Example 12a) and 6-((3S,4S)-4-amino-3-methyl-2-oxa-8-azaspiro[4.5]decan-8-yl)-5-bromo-3-(Sₐ)-(2,3-dichlorophenyl)-2-methylpyrimidin-4(3H)-one (Example 12b

5-Bromo-3-(2,3-dichlorophenyl)-6-hydroxy-2-methylpyrimidin-4(3H)-one (80 mg, 0.23 mmol), (3S,4S)-3-methyl-2-oxa-8-azaspiro[4.5]decan-4-amine (67 mg, 0.27 mmol), BOP (121 mg, 0.27 mmol) and DBU (174 mg, 1.14 mmol) in MeCN (2 mL) was stirred at rt for 2 h. The mixture was concentrated and purified by prep-HPLC (NH₄HCO₃) to afford a solid. The mixture of diastereomers were separated by Chiral SFC (Mobile phase: CO₂/methanol (0.2% MEA)=30/70; Flow rate: 80 g/min; 5 min; Column temperature: 35° C.; Back pressure: 100 bar; Column: AD 20*250 mm, 10 um (Daicel) to afford Example 12a as white solid (18 mg, 16%) and Example 12b as a white solid (15 mg, 13%).

Example 12a 6-((3S,4S)-4-Amino-3-methyl-2-oxa-8-azaspiro[4.5]decan-8-yl)-5-bromo-3-(Sₐ)-(2,3-dichlorophenyl)-2-methylpyrimidin-4(3H)-one: MS (ES+) C₂₀H₂₃BrCl₂N₄O₂ requires: 500, 502, found: 501, 503 [M+H]⁺; 1H NMR (500 MHz, DMSO) δ 7.83 (dd, J=8.1, 1.5 Hz, 1H), 7.62 (dd, J=8.0, 1.5 Hz, 1H), 7.56 (t, J=8.0 Hz, 1H), 4.09-4.02 (m, 1H), 3.93-3.82 (m, 2H), 3.66 (d, J=8.5 Hz, 1H), 3.50-3.36 (m, 3H), 2.91 (d, J=5.1 Hz, 1H), 2.00 (s, 3H), 1.86-1.78 (m, 1H), 1.68-1.59 (m, 1H), 1.61-1.47 (m, 2H), 1.08 (d, J=6.4 Hz, 3H).

Example 12b 6-((3S,4S)-4-Amino-3-methyl-2-oxa-8-azaspiro[4.5]decan-8-yl)-5-bromo-3-(Rₐ)-(2,3-dichlorophenyl)-2-methylpyrimidin-4(3H)-one: MS (ES+) C₂₀H₂₃BrCl₂N₄O₂ requires: 500, 502, found: 501, 503 [M+H]⁺; 1H NMR (500 MHz, DMSO) δ 7.83 (dd, J=8.0, 1.6 Hz, 1H), 7.62 (dd, J=7.9, 1.5 Hz, 1H), 7.56 (t, J=8.0 Hz, 1H), 4.10-4.00 (m, 1H), 3.94-3.82 (m, 2H), 3.66 (d, J=8.4 Hz, 1H), 3.53-3.36 (m, 3H), 2.91 (d, J=5.0 Hz, 1H), 2.00 (s, 3H), 1.81 (ddd, J=13.0, 9.3, 3.4 Hz, 1H), 1.69 (ddd, J=12.9, 9.1, 3.6 Hz, 1H), 1.61-1.46 (m, 2H), 1.08 (d, J=6.4 Hz, 3H).

### Example 13a and 13b

### 6-((3S,4S)-4-Amino-3-methyl-2-oxa-8-azaspiro[4.5]decan-8-yl)-3-(Sₐ)-(2-chloro-3-methylphenyl)-2-methylpyrimidin-4(3H)-one TFA salt (Example 13a

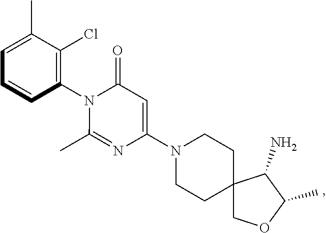

and

### 6-((3S,4S)-4-amino-3-methyl-2-oxa-8-azaspiro[4.5]decan-8-yl)-3-(Rₐ)-(2-chloro-3-methylphenyl)-2-methylpyrimidin-4(3H)-one TFA salt (Example 13b

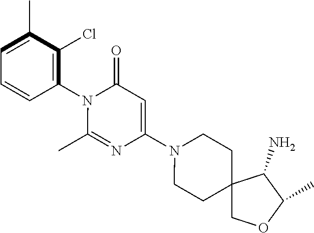

### Step 1: Tert-butyl 2-chloro-3-methylphenylcarbamate

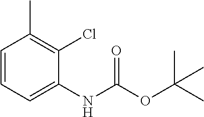 [SMILES: Cc1cccc(NC(=O)OC(C)(C)C)c1Cl]

A mixture of 1-bromo-2-chloro-3-methylbenzene (4.0 g, 19.6 mmol), Pd₂dba₃ (1014 mg, 0.98 mmol), Xantphos (566 mg, 0.98 mmol), tert-butyl carbamate (3.41 g, 29.4 mmol) and Cs₂CO₃ (9.58 g, 29.4 mmol) in dioxane (120 mL) was stirred at 110° C. for 2 h and then cooled to rt. Water (150 mL) was added and the mixture was extracted by EtOAc (150 mL×2). The organic layer was dried and concentrated. The residue was purified by flash column chromatography (PE, 100%) to afford the title compound (4.2 g, 89%). MS (ES+)Cl₂H₁₆ClNO₂ requires: 241, found: 187 [M−55+H]⁺.

### Step 2: 2-Chloro-3-methylaniline

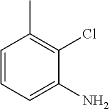

A mixture of tert-butyl 2-chloro-3-methylphenylcarbamate (4.2 g, 17.4 mmol) in 2M HCl (40 mL in MeOH) was stirred at rt for 1 h. The mixture was concentrated, washed with Et₂O (40 mL), and dried under vacuum to afford the title compound as a yellow solid (2.3 g, 93%). MS (ES+) C₇H₈ClN requires: 141, found: 142 [M+H]⁺.

### Step 3: N-(2-Chloro-3-methylphenyl)acetimidamide

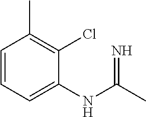

A mixture of 2-chloro-3-methylaniline (1.4 g, 9.93 mmol) in HCl (1N in CH₃CN, 15 ml) was heated at 120° C. for 4 h. The reaction mixture was cooled to rt, it was filtered and the filter cake was washed by Et₂O (15 mL). The filter cake was dried under vacuum to afford the title compound (1.2 g, 66%). MS (ES+) C₉H₁₁ClN₂ requires: 182, found: 183 [M+H]⁺.

### Step 4: 3-(2-Chloro-3-methylphenyl)-6-hydroxy-2-methylpyrimidin-4(3H)-one

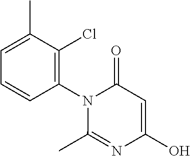 [SMILES: Cc1cccc(-n2c(C)nc(O)cc2=O)c1Cl]

To a solution of N-(2-chloro-3-methylphenyl)acetimidamide (1.2 g, 6.6 mmol) in 2-methoxyethan-1-ol (12 mL) was added diethyl malonate (4.22 g, 26.36 mmol) and CH₃ONa/CH₃OH (4M, 6.6 mL, 26 mmol). The mixture was heated at 120° C. for 16 h. The mixture was cooled to rt, it was poured into water (50 mL) and the aqueous phase was washed with Et₂O (50 mL). The aqueous layer was acidified to pH-2 with 6N HCl and it was extracted with EtOAc (50 mL×3). The organic layer was dried and concentrated. The residue was washed with EtOAc to afford the title compound (300 mg, 18%) as a white solid. MS (ES+) C₁₂H₁₁ClN₂O₂ requires: 250, found: 251[M+H]⁺.

The compound of Step 4 can exist as a mixture of 3-(Sₐ) (2-chloro-3-methylphenyl)-6-hydroxy-2-methylpyrimidin-4(3H)-one and 3-(Rₐ) (2-chloro-3-methylphenyl)-6-hydroxy-2-methylpyrimidin-4(3H)-one.

### Step 5: 6-((3S,4S)-4-Amino-3-methyl-2-oxa-8-azaspiro[4.5]decan-8-yl)-3-(Sₐ)-(2-chloro-3-methylphenyl)-2-methylpyrimidin-4(3H)-one TFA salt (Example 13a) and 6-((3S,4S)-4-amino-3-methyl-2-oxa-8-azaspiro[4.5]decan-8-yl)-3-(Rₐ)-(2-chloro-3-methylphenyl)-2-methylpyrimidin-4(3H)-one TFA salt (Example 13b

To a suspension of 3-(2-chloro-3-methylphenyl)-6-hydroxy-2-methylpyrimidin-4(3H)-one (150 mg, 0.6 mmol) and (3S,4S)-3-methyl-2-oxa-8-azaspiro[4.5]decan-4-amine dihydrochloride (173 mg, 0.72 mmol) in MeCN (3 ml) were added DBU (183 mg, 1.2 mmol) and BOP (318 mg, 0.72 mmol) and the resulting mixture was stirred at rt for 1 h. The mixture was concentrated under reduced pressure. The residue was purified by prep-HPLC (A: water (0.05% TFA), B: ACN), 18-28% B in 10 min, stop at 15 min. Column: Sunfire prep C18 10 m, OBD 19*250 mm) to afford the title compound TFA salt Example 13a, (40 mg, 25%) and the title compound TFA salt Example 13b (60 mg, 38%).

Example 13a 6-((3S,4S)-4-Amino-3-methyl-2-oxa-8-azaspiro[4.5]decan-8-yl)-3-(Sₐ)-(2-chloro-3-methylphenyl)-2-methylpyrimidin-4(3H)-one TFA salt: MS (ES+) C₂₃H₂₈ClF₃N₄O₄ requires: 402, found: 403 [M+H]⁺; 1H NMR (500 MHz, DMSO) δ 7.92 (s, 3H), 7.49-7.48 (m, 1H), 7.41-7.38 (m, 1H), 7.29-7.28 (m, 1H), 5.43 (s, 1H), 4.21-4.19 (m, 2H), 3.87-3.85 (m, 1H), 3.70-3.68 (m, 1H), 3.41-3.39 (m, 2H), 3.05-3.02 (m, 2H), 2.41 (s, 3H), 1.96 (s, 3H), 1.69-1.68 (m, 3H), 1.55-1.53 (m, 1H), 1.21-1.20 (d, J=7 Hz, 3H).

Example 13b 6-((3S,4S)-4-Amino-3-methyl-2-oxa-8-azaspiro[4.5]decan-8-yl)-3-(Rₐ)-(2-chloro-3-methylphenyl)-2-methylpyrimidin-4(3H)-one TFA salt: MS (ES+) C₂₃H₂₈ClF₃N₄O₄ requires: 402, found: 403 [M+H]⁺; 1H NMR (500 MHz, DMSO) δ 8.01 (s, 3H), 7.49-7.47 (m, 1H), 7.41-7.38 (m, 1H), 7.29-7.28 (m, 1H), 5.43 (s, 1H), 4.22-4.19 (m, 2H), 3.88-3.86 (m, 2H), 3.69-3.68 (m, 1H), 3.42-3.40 (m, 1H), 3.06-3.00 (m, 2H), 2.41 (s, 3H), 1.96 (s, 3H), 1.71-1.68 (m, 3H), 1.54-1.51 (m, 1H), 1.22-1.21 (d, J=6 Hz, 3H).

### Example 14a and 14b

### 6-((3S,4S)-4-Amino-3-methyl-2-oxa-8-azaspiro[4.5]decan-8-yl)-3-(Sₐ)-(2-chloro-3-(trifluoromethyl)phenyl)-2-methylpyrimidin-4(3H)-one TFA salt (Example 14a

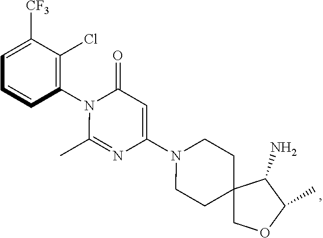

and

### 6-((3S,4S)-4-Amino-3-methyl-2-oxa-8-azaspiro[4.5]decan-8-yl)-3-(Rₐ)-(2-chloro-3-(trifluoromethyl)phenyl)-2-methylpyrimidin-4(3H)-one TFA salt (Example 14b

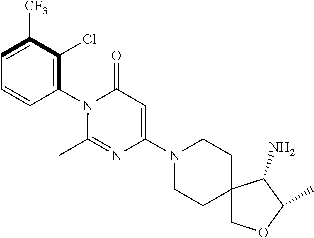

Examples 14a and 14b were synthesized with synthetic methods that were similar to that used for Examples 13a and 13b, and can generally be made by methods disclosed herein. The Examples may be made as free bases or as TFA salts.

Example 14a 6-((3S,4S)-4-Amino-3-methyl-2-oxa-8-azaspiro[4.5]decan-8-yl)-3-(Sₐ)-(2-chloro-3-(trifluoromethyl)phenyl)-2-methylpyrimidin-4(3H)-one TFA salt: MS (ES+) C₂₁H₂₄ClF₃N₄O₂ requires: 456, found: 457 [M+H]⁺.

Example 14b 6-((3S,4S)-4-Amino-3-methyl-2-oxa-8-azaspiro[4.5]decan-8-yl)-3-(Rₐ)-(2-chloro-3-(trifluoromethyl)phenyl)-2-methylpyrimidin-4(3H)-one TFA salt: MS (ES+) C₂₁H₂₄ClF₃N₄O₂ requires: 456, found: 457 [M+H]⁺.

### Example 15a and 15b

### 6-((3S,4S)-4-Amino-3-methyl-2-oxa-8-azaspiro[4.5]decan-8-yl)-3-(Sₐ)-(2,3-dichloro-4-phenoxyphenyl)-2-methylpyrimidin-4(3H)-one (Example 15a

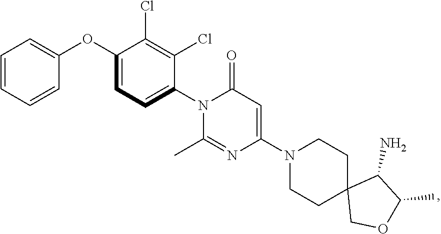

and

### 6-((3S,4S)-4-amino-3-methyl-2-oxa-8-azaspiro[4.5]decan-8-yl)-3-(Rₐ)-(2,3-dichloro-4-phenoxyphenyl)-2-methylpyrimidin-4(3H)-one (Example 15b

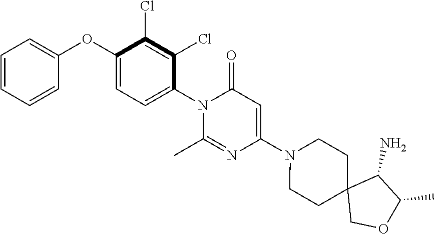

### Step 1: 2,3-Dichloro-1-nitro-4-phenoxybenzene

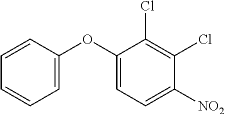 [SMILES: O=[N+]([O-])c1ccc(Oc2ccccc2)c(Cl)c1Cl]

To a solution of phenol (1.1 g, 12 mmol) in dry N,N-dimethylformamide (15 mL) was added NaH (520 mg, 13 mmol, 60%) by small portions at 0° and the resulting mixture was stirred at 0° C. for 10 min. 2,3-Dichloro-1-fluoro-4-nitrobenzene (2.1 g, 10 mmol) was added and the mixture was warmed to rt and stirred overnight. The mixture was purified by silica gel chromatography (EtOAc in PE, 0 to 15%) to afford the title compound (2.0 g, 67%) as a yellow solid. MS (ES+) C₁₂H₇Cl₂NO₃ requires 282; found: 283.

### Step 2: 2,3-Dichloro-4-phenoxyaniline

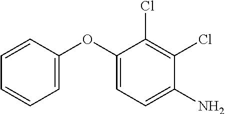 [SMILES: Nc1ccc(Oc2ccccc2)c(Cl)c1Cl]

2,3-Dichloro-1-nitro-4-phenoxybenzene (1.8 g, 6.4 mmol) and SnCl₂·2H₂O (14.3 g, 64 mmol) in EtOH (20 mL) was stirred at 85° C. for 5 h. The resulting mixture was adjusted to pH>9 with aq. sat. NaHCO₃, the solid was filtered and washed with EtOAc (50 mL×3). The organic phase was dried, concentrated under reduced pressure and purified by silica gel chromatography (EtOAc in PE, 0 to 40%) to afford the title compound (1.2 g, 73%) as a tan solid. MS (ES+) C₁₂H₉Cl₂NO requires 252; found: 253.

### Step 3: N-(2,3-Dichloro-4-phenoxyphenyl)acetimidamide

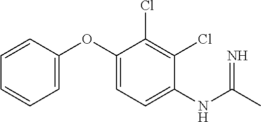 [SMILES: CC(=N)Nc1ccc(Oc2ccccc2)c(Cl)c1Cl]

2,3-Dichloro-4-phenoxyaniline (1.0 g, 3.9 mmol) in HCl/MeCN (1M, 20 mL) was heated in a sealed tube at 120° C. overnight. The mixture was cooled to rt, the solid was filtered and washed with MeCN (5 mL) to afford the title compound (0.9 g, 70%) as a white solid. MS (ES+) C₁₄H₁₂Cl₂N₂ requires 293, found: 294.

### Step 4: 3-(2,3-dichloro-4-phenoxyphenyl)-6-hydroxy-2-methylpyrimidin-4(3H)-one

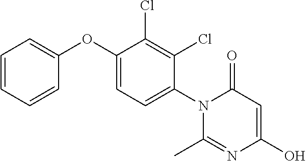 [SMILES: Cc1nc(O)cc(=O)n1-c1ccc(Oc2ccccc2)c(Cl)c1Cl]

The reaction was performed as described for Example 1, Step 2, Method B to provide the title compound (0.5 g, 54%) as a tan solid. MS (ES+) C₁₇H₁₂Cl₂N₂O₃ requires 361; found 362.

The compound of Step 4 can exist as a mixture of 3-(Sₐ)-(2,3-dichloro-4-phenoxyphenyl)-6-hydroxy-2-methylpyrimidin-4(3H)-one and 3-(Rₐ)-(2,3-dichloro-4-phenoxyphenyl)-6-hydroxy-2-methylpyrimidin-4(3H)-one.

### Step 5: 6-((3S,4S)-4-Amino-3-methyl-2-oxa-8-azaspiro[4.5]decan-8-yl)-3-(Sₐ)-(2,3-dichloro-4-phenoxyphenyl)-2-methylpyrimidin-4(3H)-one (Example 15a) and 6-((3S,4S)-4-amino-3-methyl-2-oxa-8-azaspiro[4.5]decan-8-yl)-3-(Rₐ)-(2,3-dichloro-4-phenoxyphenyl)-2-methylpyrimidin-4(3H)-one (Example 15b

The reaction was performed as described for Example 2, Step 1. The mixture of diastereomers were separated by Chiral SFC chiral-HPLC (co-solvent: MeOH (0.2% methanol-ammonia), Column: AD-H, 4.6×100 mm) to provide title compounds Example 15a (25 mg, 18%) and 15b (27 mg, 19%).

Example 15a 6-((3S,4S)-4-Amino-3-methyl-2-oxa-8-azaspiro[4.5]decan-8-yl)-3-(Sₐ)-(2,3-dichloro-4-phenoxyphenyl)-2-methylpyrimidin-4(3H)-one: MS (ES+) C₂₆H₂₈Cl₂N₄O₃ requires: 514, found: 515 [M+H]⁺; ¹H NMR (400 MHz, MeOD) δ 7.44 (m, 2H), 7.37 (dd, J=9, 2 Hz, 1H), 7.24 (t, J=7.5 Hz, 1H), 7.12 (dd, J=9, 1 Hz, 1H), 7.07 (dd, J=9, 2 Hz, 1H), 5.51 (s, 1H), 4.25 (m, 1H), 4.07 (m, 2H), 3.88 (d, J=9 Hz, 1H), 3.74 (m, 1H), 3.73 (d, J=9 Hz, 1H), 3.42 (m, 1H), 3.03 (d, J=5 Hz, 1H), 2.13 (s, 3H), 1.83-1.67 (m, 4H), 1.23 (d, J=6 Hz, 3H).

Example 15b 6-((3S,4S)-4-amino-3-methyl-2-oxa-8-azaspiro[4.5]decan-8-yl)-3-(Rₐ)-(2,3-dichloro-4-phenoxyphenyl)-2-methylpyrimidin-4(3H)-one: MS (ES+) C₂₆H₂₈Cl₂N₄O₃ requires 514; found: 515 [M+H]⁺; 1H NMR (500 MHz, MeOD) δ 7.51-7.41 (m, 2H), 7.36 (dd, J=8.8, 2.7 Hz, 1H), 7.24 (t, J=7.4 Hz, 1H), 7.12 (dd, J=8.6, 0.8 Hz, 2H), 7.07 (dd, J=8.8, 1.8 Hz, 1H), 5.51 (d, J=7.5 Hz, 1H), 4.30-4.21 (m, 1H), 4.07 (d, J=4.8 Hz, 1H,) 4.07 (m, 1H), 3.89 (d, J=8.7 Hz, 1H), 3.74 (d, J=8.7 Hz, 1H), 3.42 (t, J=10.0 Hz, 1H), 3.03 (d, J=5.0 Hz, 1H), 2.14 (d, J=3.9 Hz, 3H), 1.89-1.61 (m, 4H), 1.23 (dd, J=11.7, 6.4 Hz, 3H).

### Example 16a and 16b

### 6-((3S,4S)-4-Amino-3-methyl-2-oxa-8-azaspiro[4.5]decan-8-yl)-3-(Sₐ)-(2,3-dichloro-4-((1-methyl-1H-pyrazol-3-yl)oxy)phenyl)-2-methylpyrimidin-4(3H)-one TFA salt (Example 16a

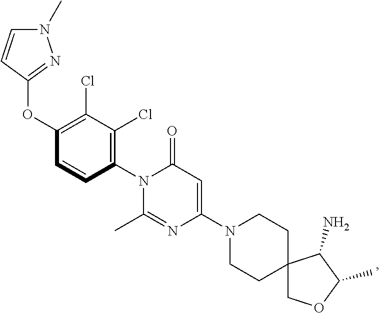

and

### 6-((3S,4S)-4-Amino-3-methyl-2-oxa-8-azaspiro[4.5]decan-8-yl)-3-(Rₐ)-(2,3-dichloro-4-((1-methyl-1H-pyrazol-3-yl)oxy)phenyl)-2-methylpyrimidin-4(3H)-one TFA salt (Example 16b

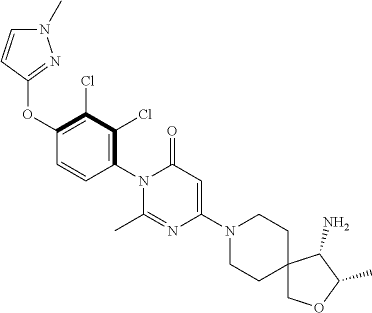

Examples 16a and 16b were synthesized with synthetic methods that were similar to that used for Examples 15a and 15b, and can generally be made by methods disclosed herein. The Examples may be made as free bases or as TFA salts.

Example 16a 6-((3S,4S)-4-Amino-3-methyl-2-oxa-8-azaspiro[4.5]decan-8-yl)-3-(Sₐ)-(2,3-dichloro-4-((1-methyl-1H-pyrazol-3-yl)oxy)phenyl)-2-methylpyrimidin-4(3H)-one TFA salt: MS (ES+) C₂₄H₂₈Cl₂N₆O₃ requires: 518, found: 519 [M+H]⁺.

Example 16b 6-((3S,4S)-4-Amino-3-methyl-2-oxa-8-azaspiro[4.5]decan-8-yl)-3-(Rₐ)-(2,3-dichloro-4-((1-methyl-1H-pyrazol-3-yl)oxy)phenyl)-2-methylpyrimidin-4(3H)-one TFA salt: MS (ES+) C₂₄H₂₈Cl₂N₆O₃ requires: 518, found: 519 [M+H]⁺.

### Example 17

### 6-((3S,4S)-4-Amino-3-methyl-2-oxa-8-azaspiro[4.5]decan-8-yl)-5-chloro-3-(2,3-dichloropyridin-4-yl)-2-methylpyrimidin-4(3H)-one

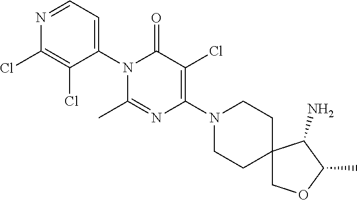 [SMILES: Cc1nc(N2CCC3(CC2)CO[C@@H](C)[C@H]3N)c(Cl)c(=O)n1-c1ccnc(Cl)c1Cl]

### Step 1: N-(2,3-Dichloropyridin-4-yl)acetimidamide

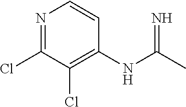

To a solution of 2,3-dichloro-4-iodopyridine (1 g mg, 3.6 mmol) in DMF (5 ml) were added acetimidamide hydrochloride (345 mg, 3.65 mmol), Cs₂CO₃ (2.4 g, 7.4 mmol) and Cu(I)I (70 mg, 0.36 mmol) and the resulting mixture was stirred at 90° C. for 12 h. The reaction mixture was diluted with MeCN (20 ml), filtered through Celite, and the filtrate was concentrated under reduced pressure to provide the title compound as a gray solid (0.83 g, 100%).

### Step 2: 3-(2,3-Dichloropyridin-4-yl)-6-hydroxy-2-methylpyrimidin-4(3H)-one

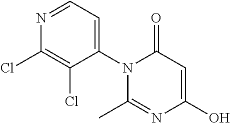 [SMILES: Cc1nc(O)cc(=O)n1-c1ccnc(Cl)c1Cl]

To a suspension of N-(2,3-dichloropyridin-4-yl)acetimidamide(1) (500 mg, 2.45 mmol) in THF (5 ml) was added bis(2,4,6-trichlorophenyl) malonate (1.2 g mg, 2.6 mmol) and the resulting mixture was stirred at 90° C. for 2 h. The volatiles were removed under reduced pressure. The reaction mixture was diluted with EtOAc (20 mL) and washed with water (5 mL). The layers were separated, and the organic layer was washed with brine (5 mL), dried over MgSO₄, filtered and concentrated under reduced pressure. The residue was purified via silica gel chromatography (0-20% MeOH in DCM to provide the title compound (280 mg, 42% yield) as a light orange solid. MS (ES+) C₁₀H₇Cl₂N₃O₂ requires: 271, found: 272 [M+H]⁺.

The compound of Step 2 can exist as a mixture of 3-(Rₐ)-(2,3-Dichloropyridin-4-yl)-6-hydroxy-2-methylpyrimidin-4(3H)-one and 3-(Sₐ)-(2,3-dichloropyridin-4-yl)-6-hydroxy-2-methylpyrimidin-4(3H)-one.

### Step 3: 6-((3S,4S)-4-Amino-3-methyl-2-oxa-8-azaspiro[4.5]decan-8-yl)-3-(2,3-dichloropyridin-4-yl)-2-methylpyrimidin-4(3H)-one

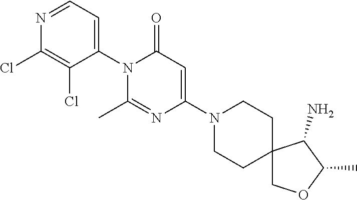 [SMILES: Cc1nc(N2CCC3(CC2)CO[C@@H](C)[C@H]3N)cc(=O)n1-c1ccnc(Cl)c1Cl]

To 3-(2,3-dichloropyridin-4-yl)-6-hydroxy-2-methylpyrimidin-4(3H)-one (100 mg, 0.37 mol), (3S,4S)-3-methyl-2-oxa-8-azaspiro[4.5]decan-4-amine dihydrochloride (89 mg, 0.37 mmol), BOP (244 mg, 0.551 mmol) in DMF (2 ml) was added DBU (0.11 ml, 0.73 mmol) and the resulting mixture was stirred at rt for 1 h. The residue was purified by mass-triggered preparative HPLC (Mobile phase: A=0.1% TFA/H₂O, B=0.1% TFA/MeCN; Gradient: B=10-50%; 20 min; Column: C18) to provide the title compound (92 mg, 0.22 mmol, 59% yield) as a white solid. MS (ES+) C₁₉H₂₃Cl₂N₅O₂ requires: 423, found: 424 [M+H]+.

The compound of Step 3 can exist as a mixture of 6-((3S,4S)-4-amino-3-methyl-2-oxa-8-azaspiro[4.5]decan-8-yl)-3-(Rₐ)-(2,3-dichloropyridin-4-yl)-2-methylpyrimidin-4(3H)-one and 6-((3S,4S)-4-amino-3-methyl-2-oxa-8-azaspiro[4.5]decan-8-yl)-3-(Sₐ)-(2,3-dichloropyridin-4-yl)-2-methylpyrimidin-4(3H)-one.

### Step 4: 6-((3S,4S)-4-Amino-3-methyl-2-oxa-8-azaspiro[4.5]decan-8-yl)-5-chloro-3-(2,3-dichloropyridin-4-yl)-2-methylpyrimidin-4(3H)-one

A vial was charged with 6-((3S,4S)-4-amino-3-methyl-2-oxa-8-azaspiro[4.5]decan-8-yl)-3-(2,3-dichloropyridin-4-yl)-2-methylpyrimidin-4(3H)-one (40 mg, 0.047 mmol), NCS (15 mg, 0.11 mmol) and DCM (1.0 ml). The mixture was stirred at rt for 5 min. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to provide the title compound (12 mg, 0.026 mmol, 11% yield) as a white powder. MS (ES+) C₁₉H₂₂Cl₃N₅O₂ requires: 457, found: 458 [M+H]⁺; ¹H NMR (500 MHz, DMSO-d₆) δ 8.64 (d, J=5.0 Hz, 1H); 7.94 (m, 2H); 7.82 (m. 1H); 4.16-4.26 (m, 2H); 3.86 (m, 1H); 3.71 (d, J=10 Hz, 1H); 3.17-3.26 (m, 3H); 2.81 (d, J=10.68 Hz, 1H), 2.06 (s, 3H), 1.73-1.81 (m, 3H), 1.62 (m, 1H), 1.22 (d, J=5.0 Hz, 3H).

The compound of Example 17 can exist as a mixture of 6-((3S,4S)-4-amino-3-methyl-2-oxa-8-azaspiro[4.5]decan-8-yl)-5-chloro-3-(Sₐ)-(2,3-dichloropyridin-4-yl)-2-methylpyrimidin-4(3H)-one and 6-((3S,4S)-4-amino-3-methyl-2-oxa-8-azaspiro[4.5]decan-8-yl)-5-chloro-3-(Rₐ)-(2,3-dichloropyridin-4-yl)-2-methylpyrimidin-4(3H)-one.

### Example 18a and 18b

### 6-((3S,4S)-4-Amino-3-methyl-2-oxa-8-azaspiro[4.5]decan-8-yl)-3-(Sₐ)-(2,3-dichloro-4-((1-methyl-1H-pyrazol-3-yl)oxy)phenyl)-2,5-dimethylpyrimidin-4(3H)-one (Example 18a

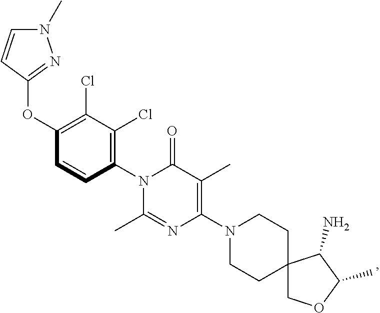

and

### 6-((3S,4S)-4-amino-3-methyl-2-oxa-8-azaspiro[4.5]decan-8-yl)-3-(Rₐ)-(2,3-dichloro-4-((1-methyl-1H-pyrazol-3-yl)oxy)phenyl)-2,5-dimethylpyrimidin-4(3H)-one (Example 18b

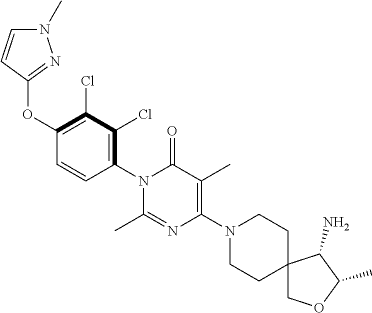

### Step 1: 3-(2,3-Dichloro-4-nitrophenoxy)-1-methyl-1H-pyrazole

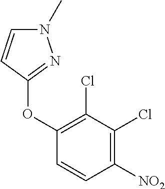 [SMILES: Cn1ccc(Oc2ccc([N+](=O)[O-])c(Cl)c2Cl)n1]

A mixture of 2,3-dichloro-1-fluoro-4-nitrobenzene (1.3 g, 6.2 mmol), 1-methyl-1,2-dihydro-3H-pyrazol-3-one (729 mg, 7.4 mmol), K₂CO₃ (1.7 g, 12.4 mmol) in MeCN (30 mL) was stirred at rt for 3 h. The reaction mixture was poured into water (50 ml) and extracted with EtOAc (100 mL×3). The combined organic phases were dried (Na₂SO₄), filtered, and concentrated and the residue was purified by silica gel chromatography (EtOAc in PE, 10%) to provide the title compound (1.5 g, 84%) as a white solid. MS (ES+) C₁₀H₇Cl₂N₃O₃ requires: 287; found: 288 [M+H]⁺.

### Step 2: 2,3-Dichloro-4-((1-methyl-1H-pyrazol-3-yl)oxy)aniline

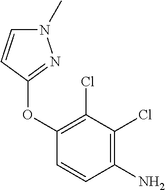 [SMILES: Cn1ccc(Oc2ccc(N)c(Cl)c2Cl)n1]

To a solution of 3-(2,3-dichloro-4-nitrophenoxy)-1-methyl-1H-pyrazole (1.5 g, 5.2 mmol) in AcOH (10 mL) was added Fe (1.4 g, 26 mmol). The resulting mixture was stirred at 60° C. for 2 h. The reaction mixture was poured into ice water (60 ml) and extracted with EtOAc (100 mL×3). The combined organic phases were dried (Na₂SO₄), filtered and concentrated. The residue was purified by silica gel chromatography (EtOAc in PE, 50%) to provide the title compound (1.2 g, 90%) as a yellow solid. MS (ES+) C₁₀H₉Cl₂N₃O requires: 257, found: 258[M+H]⁺.

### Step 3: N-(2,3-Dichloro-4-((1-methyl-1H-pyrazol-3-yl)oxy)phenyl) acetimidamide

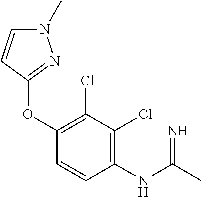 [SMILES: CC(=N)Nc1ccc(Oc2ccn(C)n2)c(Cl)c1Cl]

A mixture of 2,3-dichloro-4-((1-methyl-1H-pyrazol-3-yl)oxy)aniline (1.2 g, 4.7 mmol) in HCl/CH₃CN (1 M, 10 mL) was stirred in a seal tube at 120° C. for 24 h. The mixture was concentrated and the residue was washed with Et₂O (10 mL). The precipate was filtered to provide the title compound (1.2 g, 86%) as a white solid. MS (ES+) C₁₂H₁₂Cl₂N₄O requires: 298, found: 299[M+H]⁺.

### Step 4: 3-(2,3-Dichloro-4-((1-methyl-1H-pyrazol-3-yl)oxy)phenyl)-6-hydroxy-2,5-dimethylpyrimidin-4(3H)-one

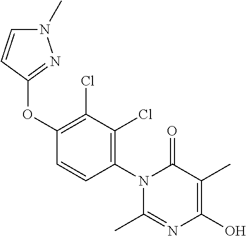 [SMILES: Cc1c(O)nc(C)n(-c2ccc(Oc3ccn(C)n3)c(Cl)c2Cl)c1=O]

To a solution of diethyl 2-methylmalonate (1.4 g, 8.0 mmol) in EtOH (6 mL) was added CH₃ONa/CH₃OH (4 M, 2 mL). The mixture was stirred at rt for 10 min. N-(2,3-Dichloro-4-((1-methyl-1H-pyrazol-3-yl)oxy)phenyl)acetimidamide (600 mg, 2 mmol) was added and the mixture was stirred at 120° C. for 4 h. The mixture was poured into water (30 mL) and extracted with Et₂O (30 mL×2). The pH of the aqueous layer was adjusted pH 5 by AcOH and extracted with EtOAc (80 mL×3). The combined organic phases were dried (Na₂SO₄), filtered and concentrated. The crude product was washed with Et₂O (10 mL) to provide the title compound (320 mg, 42%) as a white solid. MS (ES+) C₁₆H₁₄Cl₂N₄O₃ requires: 380, found: 381 [M+H]⁺.

The compound of Step 4 can exist as a mixture of 3-(Sₐ)-(2,3-dichloro-4-((1-methyl-1H-pyrazol-3-yl)oxy)phenyl)-6-hydroxy-2,5-dimethylpyrimidin-4(3H)-one and 3-(Rₐ)-(2,3-dichloro-4-((1-methyl-H-pyrazol-3-yl)oxy)phenyl)-6-hydroxy-2,5-dimethylpyrimidin-4(3H)-one.

### Step 5: 6-((3S,4S)-4-Amino-3-methyl-2-oxa-8-azaspiro[4.5]decan-8-yl)-3-(Sₐ)-(2,3-dichloro-4-((1-methyl-1H-pyrazol-3-yl)oxy)phenyl)-2,5-dimethylpyrimidin-4(3H)-one (Example 18a) and 6-((3S,4S)-4-Amino-3-methyl-2-oxa-8-azaspiro[4.5]decan-8-yl)-3-(Rₐ)-(2,3-dichloro-4-((1-methyl-1H-pyrazol-3-yl)oxy)phenyl)-2,5-dimethylpyrimidin-4(3H)-one (Example 18b

To a suspension of 3-(2,3-dichloro-4-((1-methyl-1H-pyrazol-3-yl)oxy)phenyl)-6-hydroxy-2,5-dimethylpyrimidin-4(3H)-one (100 mg, 0.26 mmol) and (3S,4S)-3-methyl-2-oxa-8-azaspiro[4.5]decan-4-amine dihydrochloride (126 mg, 0.52 mmol) in MeCN (8 ml) was added DBU (395 mg, 2.6 mmol) and BOP (230 mg, 0.52 mmol) and the resulting mixture was stirred at rt for 48 h. The mixture was concentrated under reduced pressure and the residue was purified by preparative HPLC (Mobile phase: A=0.05% TFA/H₂O, B=MeCN; Gradient: B=21-31%; 10 min; Column: C18) and the diastereomers were separated using a Chiralpak OZ Column (20×250 mm) with CO₂ and 0.2% methanol ammonia (45:55) as an eluent, a flow rate of 80 g/min and UV detection (214 nm) to provide Example 18a (7 mg, 5%) and Example 18b (9 mg, 7%) as a white solid.

Example 18a 6-((3S,4S)-4-Amino-3-methyl-2-oxa-8-azaspiro[4.5]decan-8-yl)-3-(Sₐ)-(2,3-dichloro-4-((1-methyl-1H-pyrazol-3-yl)oxy)phenyl)-2,5-dimethylpyrimidin-4(3H)-one: MS (ES+) C₂₅H₃₀Cl₂N₆O₃ requires: 532 found: 533 [M+H]⁺; ¹H NMR (500 MHz, MeOD) δ 7.46 (d, J=2.5 Hz, 1H), 7.14-7.23 (m, 2H), 5.84 (d, J=2.5 Hz, 1H), 4.12-4.15 (m, 1H), 3.71-3.78 (m, 1H), 3.62-3.68 (m, 6H), 3.17-3.20 (m, 3H), 2.95-2.96 (m, 1H), 1.99 (s, 3H), 1.90 (s, 3H), 1.57-1.78 (m, 4H), 1.18 (s, 1H), 1.12 (d, J=6 Hz, 3H).

Example 18b 6-((3S,4S)-4-Amino-3-methyl-2-oxa-8-azaspiro[4.5]decan-8-yl)-3-(Rₐ)-(2,3-dichloro-4-((1-methyl-1H-pyrazol-3-yl)oxy)phenyl)-2,5-dimethylpyrimidin-4(3H)-one: MS (ES+) C₂₅H₃₀Cl₂N₆O₃ requires: 532 found: 533 [M+H]⁺; ¹H NMR (500 MHz, MeOD) δ 7.58 (d, J=2.5 Hz, 1H), 7.26-7.36 (m, 2H), 5.96 (d, J=3 Hz, 1H), 4.24-4.26 (m, 1H), 3.83-3.89 (m, 1H), 3.74-3.80 (m, 6H), 3.18-3.29 (m, 3H), 3.05-3.06 (m, 1H), 2.69 (s, 1H), 2.11 (s, 3H), 2.02 (s, 3H), 1.66-1.88 (m, 4H), 1.23 (d, J=7 Hz, 3H).

### Example 19a and 19b

### 4-(4-(4-((3S,4S)-4-Amino-3-methyl-2-oxa-8-azaspiro[4.5]decan-8-yl)-2,5-dimethyl-6-oxopyrimidin-1(6H)-yl)-(Sₐ)-(2,3-dichlorophenoxy)-N-methylbenzamide (Example 19a

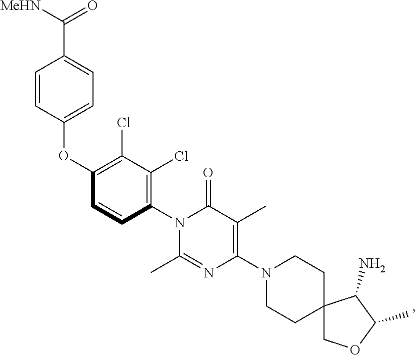

and

### 4-(4-(4-((3S,4S)-4-Amino-3-methyl-2-oxa-8-azaspiro[4.5]decan-8-yl)-2,5-dimethyl-6-oxopyrimidin-1(6H)-yl)-(Rₐ)-(2,3-dichlorophenoxy)-N-methylbenzamide (Example 19b

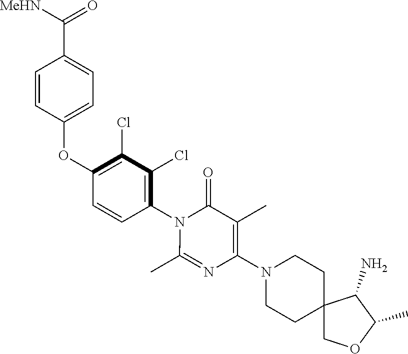

Examples 19a and 19b were synthesized with synthetic methods that were similar to that used for Examples 15a and 15b, and can generally be made by methods disclosed herein. The Examples may be made as free bases or as TFA salts.

Example 19a 4-(4-(4-((3S,4S)-4-Amino-3-methyl-2-oxa-8-azaspiro[4.5]decan-8-yl)-2,5-dimethyl-6-oxopyrimidin-1(6H)-yl)-(Sₐ)-(2,3-dichlorophenoxy)-N-methylbenzamide: MS (ES+) C₂₉H₃₃Cl₂N₅O₄ requires: 585, found: 586 [M+H]⁺.

Example 19b 4-(4-(4-((3S,4S)-4-Amino-3-methyl-2-oxa-8-azaspiro[4.5]decan-8-yl)-2,5-dimethyl-6-oxopyrimidin-1(6H)-yl)-(Rₐ)-(2,3-dichlorophenoxy)-N-methylbenzamide: MS (ES+) C₂₉H₃₃Cl₂N₅O₄ requires: 585, found: 586 [M+H]⁺.

### Example 20a and 20b

### 3-(4-((1H-Pyrazol-3-yl)oxy)-(Sₐ)-2,3-dichlorophenyl)-6-((3S,4S)-4-amino-3-methyl-2-oxa-8-azaspiro[4.5]decan-8-yl)-2,5-dimethylpyrimidin-4(3H)-one (Example 20a

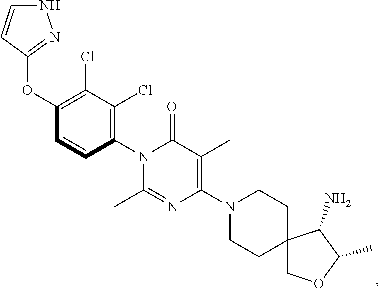

and

### 3-(4-((1H-pyrazol-3-yl)oxy)-(Rₐ)-2,3-dichlorophenyl)-6-((3S,4S)-4-amino-3-methyl-2-oxa-8-azaspiro[4.5]decan-8-yl)-2,5-dimethylpyrimidin-4(3H)-one (Example 20b

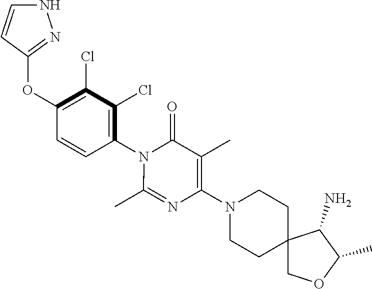

### Step 1: tert-Butyl 3-(2,3-dichloro-4-nitrophenoxy)-1H-pyrazole-1-carboxylate

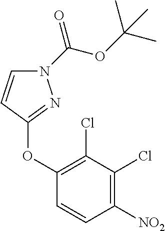 [SMILES: CC(C)(C)OC(=O)n1ccc(Oc2ccc([N+](=O)[O-])c(Cl)c2Cl)n1]

A mixture of tert-butyl 3-oxo-2,3-dihydro-1H-pyrazole-1-carboxylate (687 mg, 3.73 mmol), 2,3-dichloro-1-fluoro-4-nitrobenzene (650 mg, 3.11 mmol) and K₂CO₃ (858 mg, 6.22 mmol) in DMSO (10 mL) was stirred at rt for 4 h. Water (20 mL) was added and the mixture was extracted with EtOAc. The combined organic phases were dried over Na₂SO₄, filtered and purified by column chromatography (EtOAc in PE; 20 to 50%) to afford the title compound (800 mg, 67%) as a yellow solid. MS (ES+) C₁₄H₁₃Cl₂N₃O₅ requires: 373, found:

“\*MERGEFORMAT\*MERGEFORMAT 318 [M−56+H]⁺.

### Step 2: tert-Butyl 3-(4-amino-2,3-dichlorophenoxy)-1H-pyrazole-1-carboxylate

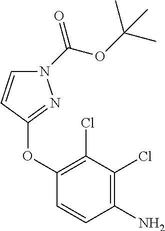 [SMILES: CC(C)(C)OC(=O)n1ccc(Oc2ccc(N)c(Cl)c2Cl)n1]

A suspension of tert-butyl 3-(2,3-dichloro-4-nitrophenoxy)-1H-pyrazole-1-carboxylate (1.2 g, 3.2 mmol), Pt/C (200 mg) in EtOH (100 mL) was stirred under H₂ atmosphere at rt for 16 h. The mixture was filtered and the filtrate was concentrated to afford the title compound (1.1 g, 100%) as a beige solid. MS (ESI+) C₁₄H₁₅Cl₂N₃O₃ requires: 343, found: 288 [M−56+H]⁺.

### Step 3: N-(4-((1H-Pyrazol-3-yl)oxy)-2,3-dichlorophenyl)acetimidamide

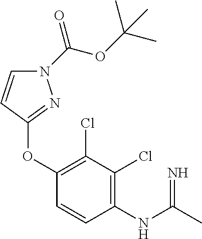 [SMILES: CC(=N)Nc1ccc(Oc2ccn(C(=O)OC(C)(C)C)n2)c(Cl)c1Cl]

A mixture of tert-butyl 3-(4-amino-2,3-dichlorophenoxy)-1H-pyrazole-1-carboxylate (1.1 g, 3.2 mmol) in HCl/CH₃CN (1 M, 20 mL) was stirred in a sealed tube at 110° C. for 4 h. The mixture was concentrated to give N-(4-((1H-pyrazol-3-yl)oxy)-2,3-dichlorophenyl)acetimidamide as a crude product. MS (ESI+) C₁₁H₁₀C₁₂N₄O requires: 284, found: 285 [M+H]⁺.

### Step 4: 3-(4-((1H-Pyrazol-3-yl)oxy)-2,3-dichlorophenyl)-6-hydroxy-2,5-dimethylpyrimidin-4(3H)-one

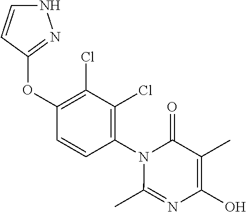 [SMILES: Cc1c(O)nc(C)n(-c2ccc(Oc3cc[nH]n3)c(Cl)c2Cl)c1=O]

A mixture of N-(4-((1H-pyrazol-3-yl)oxy)-2,3-dichlorophenyl)acetimidamide (600 mg, 2.11 mmol) diethyl 2-methylmalonate (1.84 g, 10.6 mmol) in EtOH (10 mL) was added CH₃ONa/CH₃OH (4 M, 2.64 mL). The mixture was stirred at 110° C. for 5 h. The mixture was concentrated and dissolved in water (20 mL). The aqueous layer was extracted with EtOAc (20 mL) and adjusted pH to 5 with 6 N HCl. Solid was precipitated out, it was collected by filtration to give the title compound (300 mg, 38.7%) as a beige solid. MS (ESI+) C₁₅H₁₂Cl₂N₄O₃ requires: 366, found: 367 [M+H]⁺.

The compound of Step 4 can exist as a mixture of 3-(4-((1H-pyrazol-3-yl)oxy)-(Sₐ)-2,3-dichlorophenyl)-6-hydroxy-2,5-dimethylpyrimidin-4(3H)-one and 3-(4-((1H-pyrazol-3-yl)oxy)-(Rₐ)-2,3-dichlorophenyl)-6-hydroxy-2,5-dimethylpyrimidin-4(3H)-one.

### Step 5: 3-(4-((1H-Pyrazol-3-yl)oxy)-(Sₐ)-2,3-dichlorophenyl)-6-((3S,4S)-4-amino-3-methyl-2-oxa-8-azaspiro[4.5]decan-8-yl)-2,5-dimethylpyrimidin-4(3H)-one (Example 20a) and 3-(4-((1H-pyrazol-3-yl)oxy)-(Rₐ)-2,3-dichlorophenyl)-6-((3S,4S)-4-amino-3-methyl-2-oxa-8-azaspiro[4.5]decan-8-yl)-2,5-dimethylpyrimidin-4(3H)-one (Example 20b

The experiment was performed as described for Examples 18a and 18b, step 5. The mixture was separated using a Chiralpak OZ Column (20×250 mm) with CO₂ and 0.2% methanol ammonia (50:50) as an eluent, a flow rate of 80 g/min and UV detection (214 nm) to provide Example 20a (15 mg, 11%) and Example 20b (13 mg, 11%) as a white solid.

Example 20a 3-(4-((1H-Pyrazol-3-yl)oxy)-(Sₐ)-2,3-dichlorophenyl)-6-((3S,4S)-4-amino-3-methyl-2-oxa-8-azaspiro[4.5]decan-8-yl)-2,5-dimethylpyrimidin-4(3H)-one: MS (ES+) C₂₄H₂₈Cl₂N₆O₃ requires: 518, found: 519 [M+H]*; 1H NMR (500 MHz, DMSO) δ 12.57 (d, J=0.4 Hz, 1H), 7.78 (s, 1H), 7.48 (d, J=8.9 Hz, 1H), 7.18 (d, J=8.9 Hz, 1H), 6.05 (s, 1H), 4.12-3.99 (m, 1H), 3.67 (d, J=8.4 Hz, 1H), 3.60-3.52 (m, 2H), 3.50 (d, J=8.4 Hz, 1H), 3.23-3.07 (m, 2H), 2.93 (d, J=4.9 Hz, 1H), 2.00 (s, 3H), 1.89 (s, 3H), 1.81-1.74 (m, 1H), 1.70-1.63 (m, 1H), 1.59-1.47 (m, 2H), 1.23 (s, 1H), 1.09 (d, J=6.3 Hz, 3H).

Example 20b 3-(4-((1H-Pyrazol-3-yl)oxy)-(Rₐ)-2,3-dichlorophenyl)-6-((3S,4S)-4-amino-3-methyl-2-oxa-8-azaspiro[4.5]decan-8-yl)-2,5-dimethylpyrimidin-4(3H)-one: MS (ES+) C₂₄H₂₈Cl₂N₆O₃ requires: 518, found: 519 [M+H]⁺; 1H NMR (500 MHz, DMSO) δ 12.57 (s, 1H), 7.81-7.75 (m, 1H), 7.48 (d, J=8.9 Hz, 1H), 7.18 (d, J=9.0 Hz, 1H), 6.05 (t, J=2.1 Hz, 1H), 4.10-4.01 (m, 1H), 3.67 (d, J=8.5 Hz, 1H), 3.61-3.51 (m, 2H), 3.50 (d, J=8.5 Hz, 1H), 3.23-3.18 (m, 1H), 3.15-3.06 (m, 1H), 2.93 (d, J=4.9 Hz, 1H), 2.00 (s, 3H), 1.89 (s, 3H), 1.80-1.75 (m, 1H), 1.70-1.63 (m, 1H), 1.58-1.48 (m, 2H), 1.32-1.19 (m, 2H), 1.09 (d, J=6.4 Hz, 3H).

### Example 21: Biological Activity Assay

The activity of the compounds in the Examples disclosed herein as PTPN11 inhibitors is illustrated in the following assays.

### PTPN11 Enzymatic Assay

Recombinant full-length wild-type and E76K mutant human PTPN11 proteins were cloned, expressed (E. coli system), and isolated via a two-step purification of Ni affinity followed by S75 size exclusion chromatography.

Phosphatase activity of full length wild-type PTPN11 (PTPN11-WT) or PTPN11-E76K mutant enzyme was measured using the fluorogenic 6,8-difluoro-4-methylumbelliferyl phosphate (DiFMUP; Molecular Probes) as the substrate. Enzyme (250 μM) was incubated with or without increasing concentrations of compounds in assay buffer (62.5 mM HEPES, 125 mM NaCl, 1 mM EDTA, 1.25 mM TECP, 0.1% BSA) for 30 min at room temperature. Reaction was initiated by addition of DiFMUP (50 μM) at room temperature in 384-well black plate with a final reaction volume of 20 uL in assay buffer. After 1 hour, DiFMUP fluorescence signal was measured (Ex: 340/Em: 460) using Envision plate reader. Dose-response curves were analyzed using IC₅₀ regression curve fitting (GeneData Screener). Curves were normalized to a high controls without inhibitor, and low controls without substrate. Results are provided below in Table 2.

TABLE 2
Biological Activity for inhibition of PTPN11-E76K mutant enzyme

| Example # | IC₅₀, nM |
|---|---|
| 1 | 510 |
| 2a | 7047 |
| 2b | 122 |
| 3 | 310 |
| 4 | 480 |
| 5 | 1172 |
| 6 | 203 |
| 7a | >10000 |
| 7b | 153 |
| 8 | 360 |
| 9 | 45 |
| 10a | 2583 |
| 10b | 28 |
| 11 | 366 |
| 12a | 3457 |
| 12b | 20 |
| 13a | 7500 |
| 13b | 186 |
| 14a | >1000 |
| 14b | 703 |
| 15a | >1000 |
| 15b | 148 |
| 16a | 9552 |
| 16b | 92 |
| 17 | 618 |
| 18a | 511 |
| 18b | 3 |
| 19b | 12 |
| 20b | 5 |

### ERK Phosphorylation (Phospho-ERK) Target Engagement Assay

KYSE-520 cells (10 k cells/well) are plated onto 384-well plate in 20 μL of medium (RPMI-1640, without phenol red, containing 10% FBS) and incubated at 37° C., 5% CO₂ 16 h. DMSO (control) or increasing concentrations of compounds are diluted in medium, added to the 384-well plate (10 uL/well, final DMSO concentration of 1%), and cells are then incubated with compounds for 2 hr. Phospho-ERK levels are measured using a TR-FRET based phospho-ERK1/2 HTRF kit (CisBio, 64ERKPEH) following manufacturer's recommendations, and fluorescence signal was measured at 665 nm and 620 nm using Synergy Neo plate reader. Dose-response curves were analyzed using IC₅₀ regression curve fitting (GeneData Screener). Curves were normalized to a high controls without inhibitor, and low controls with 1 μM of selumetinib. Some compounds of this invention showed IC₅₀<1 μM.

### Colony Formation Assay

KYSE-520 cells (2000 cells/well) are plated in 6-well plate containing 2 mL of medium (RPMI-1640, containing 10% FBS), in presence of DMSO (control; 1% final concentration) or increasing compound concentration. After 14 days of culture at 37° C. in a humidified 5% CO₂ incubator, colonies are fixed and stained with 0.1% crystal violet and 15% ethanol solution. Plates are imaged and colony area quantified and normalized to DMSO with ImageJ, Colony Area plugin. (Guzmán, Camilo, PloS one 2014). Some compounds of this invention displayed IC₅₀<1 μM).

### Example 22: In Vivo Studies

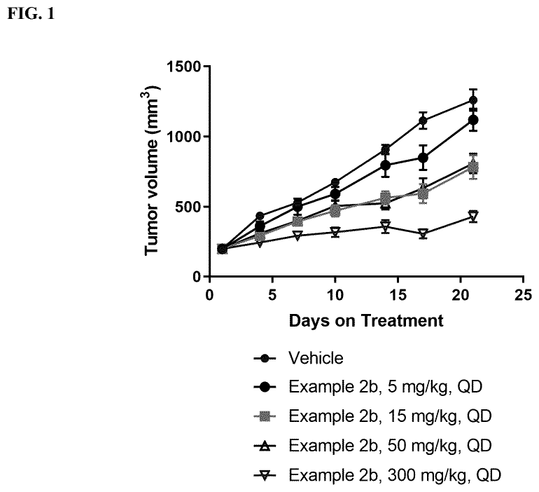

The in vivo anti-tumor efficacy of Example 2b was evaluated in NSG (NOD scid gamma) mice with subcutaneously implanted KYSE520 xenografts. Example 2b was administered by oral gavage once a day for 21 days at dosing levels of 300 mg/kg, 50 mg/kg, 15 mg/kg and 5 mg/kg. Example 2b exhibited a dose-dependent tumor growth inhibition in the KYSE520 xenograft model at tolerated dose, as shown in FIG. 1.

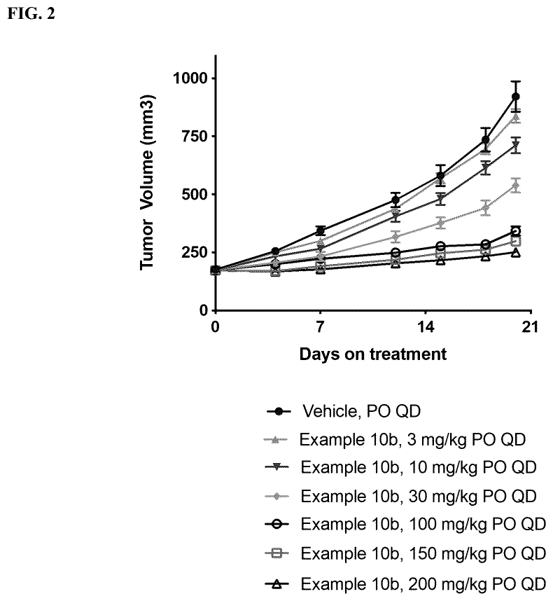

The in vivo anti-tumor efficacy of Example 10b was evaluated in NSG mice with subcutaneously implanted KYSE520 xenografts. Example 10b was administered by oral gavage once a day for 21 days at 200 mg/kg, 150 mg/kg, 100 mg/kg, 30 mg/kg, 10 mg/kg and 3 mg/kg. Example 10b exhibited a dose-dependent tumor growth inhibition in the KYSE520 xenograft model at tolerated dose, as shown in FIG. 2.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

### VIII. EMBODIMENTS

For further illustration, additional non-limiting embodiments of the present invention are set forth below.

Embodiment 1 is a compound represented by Formula I

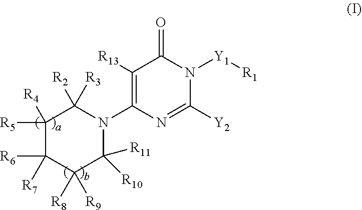

or a salt, ester, or prodrug thereof, wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—, C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one to five groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O)ₘ;
subscript m is 0, 1, or 2;
said saturated or unsaturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices elected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆alkyl, C₁₋₆haloalkyl, C₁₋₆hydroxyalkyl, C₁₋₆ dihydroxyalkyl, —NH—NHR₁₉, —NHR₁₉, —OR₁₉, —NHC(O)R₁₉, —NHC(O)NHR₁₉, —NHS(O)₂NHR₁₉, —NHS(O)₂R₁₉, —C(O)OR₁₉, —C(O)NR₁₉R₂₀, —C(O)NH(CH₂)_{q}OH, —C(O)NH(CH₂)_{q}R₂₁, —C(O)R₂₁, —NH₂, —OH, —S(O)₂NR₁₉R₂₀, C₃₋₈cycloalkyl, aryl, heterocyclyl having 1-5 heteroatoms as ring vertices selected from N, O, S and P, heteroaryl having 1-5 heteroatoms as ring vertices selected from N, O, S and P; wherein the subscript q is an integer of from 0 to 6; and wherein aryl, heteroaryl, heterocyclyl and cycloalkyl are substituted with 0 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, each of which is optionally substituted by one or more groups independently selected from the group consisting of amido, amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆ alkyl, C₁₋₆haloalkyl, C₁₋₆hydroxyalkyl, C₂₋₆alkenyl, C₂₋₆alkynyl and C₃₋₆cycloalkyl.

Embodiment 2 is the compound of embodiment 1, wherein:
subscript a is 0 or 1;
subscript b is 0 or 1;
Y₁ is a direct bond or CR₁₇R₁₈;
Y₂ is selected from the group consisting of C₁₋₄alkyl, amino, C₁₋₄alkylC(O)O—, C₁₋₄alkylamino and C₁₋₄aminoalkyl;
R₁ is selected from the group consisting of C₆₋₁₀aryl, C₃₋₈cycloalkyl, C₃₋₈cycloalkenyl, and a 5-10 membered heteroaryl group having 1 to 4 heteroatoms or groups as ring vertices independently selected from N, C(O), O, and S; said aryl or heteroaryl of R₁ is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄;
R₂, R₃, R₁₀, and R₁₁ are each independently selected from the group consisting of hydrogen, C₁₋₄alkyl, and C₃₋₈cycloalkyl;
R₄, R₅, R₈, and R₉ are each independently selected from the group consisting of hydrogen, cyano, C₁₋₄alkyl, C₁₋₄alkoxy, amino, hydroxy, C₃₋₈cycloalkyl, halo, and C₁₋₄alkylamino;
R₆ is selected from the group consisting of amino, C₁₋₄aminoalkyl, and C₁₋₄alkylamino;
R₇ is selected from the group consisting of hydrogen, halo, and hydroxy, or is selected from the group consisting of amido, C₁₋₄alkyl, C₁₋₄hydroxyalkyl, C₃₋₆cycloalkyl, phenyl, and 5- or 6-membered heteroaryl, any of which is optionally substituted with one to five groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino, and C₁₋₄aminoalkyl;
or R₆ and R₇ together with the carbon atom to which they are both attached form a 3- to 7-membered saturated or unsaturated ring, having 0 to 3 heteroatoms or groups as ring vertices independently selected from N, C(O), O, and S(O)ₘ;
subscript m is 0, 1, or 2;
said saturated ring formed by R₆ and R₇ is unsubstituted or substituted with 1 to 3 groups independently selected from the group consisting of amino, halo, hydroxy, cyano, C₁₋₄alkyl, C₁₋₄alkoxy, C₁₋₄haloalkyl, C₁₋₄haloalkoxy, C₁₋₄alkylamino and C₁₋₄aminoalkyl;
any two groups of R₂, R₃, R₄, R₅, R₇, R₈, R₉, R₁₀ and R₁₁ can form a 5 to 6 membered ring, having 0 to 2 heteroatoms as ring vertices selected from N, O and S;
any two groups of R₂, R₄, R₆, R₈ and R₁₀ can form a direct bond, or a 1 or 2 atom carbon bridge;
R₁₃ is selected from the group consisting of hydrogen, halo, cyano, C₁₋₆ alkyl, C₁₋₆haloalkyl, C₁₋₆hydroxyalkyl, C₁₋₆dihydroxyalkyl, —NH—NHR₁₉, —NHR₁₉, —OR₁₉, —NHC(O)R₁₉, —NHC(O)NHR₁₉, —NHS(O)₂NHR₁₉, —NHS(O)₂R₁₉, —C(O)OR₁₉, —C(O)NR₁₉R₂₀, —C(O)NH(CH₂)_{q}OH, —C(O)NH(CH₂)_{q}R₂₁, —C(O)R₂₁, —NH₂, —OH, —S(O)₂NR₁₉R₂₀, C₃₋₈cycloalkyl, aryl, heterocyclyl having 1-5 heteroatoms as ring vertices selected from N, O, S and P, heteroaryl having 1-5 heteroatoms as ring vertices selected from N, O, S and P; wherein the subscript q is an integer of from 0 to 6; and wherein aryl, heteroaryl, heterocyclyl and cycloalkyl are substituted with 0 to 3 groups independently selected from the group consisting of C₁₋₄alkyl, —OH, —NH₂, —OR₂₁, halo, cyano, and oxo;
R₁₄, R₁₅ and R₁₆ are each independently selected from the group consisting of hydrogen, C₁₋₄alkyl, C₃₋₈cycloalkyl, C₆₋₁₀aryl and 5-10 membered heteroaryl, each of which is optionally substituted by one or more groups independently selected from the group consisting of halo, hydroxy, cyano, and C₁₋₄alkyl;
R₁₇ and R₁₈ are each independently selected from the group consisting of hydrogen, C₁₋₄alkyl, and CF₃;
R₁₉ and R₂₀ are each independently selected from the group consisting of hydrogen, C₁₋₆alkyl, C₁₋₆haloalkyl, C₁₋₆hydroxyalkyl, C₂₋₆alkenyl, C₂₋₆alkynyl and C₃₋₆cycloalkyl; and
each R₂₁ is independently selected from the group consisting of hydrogen, —OH, C₁₋₆ alkyl, C₁₋₆haloalkyl, C₁₋₆hydroxyalkyl, C₂₋₆alkenyl, C₂₋₆alkynyl and C₃₋₆cycloalkyl.

Embodiment 3 is the compound of embodiment 1 or 2, wherein subscripts a and b are each 1.

Embodiment 4 is the compound of any one of embodiments 1 to 3, wherein Y₁ is a direct bond.

Embodiment 5 is the compound of any one of embodiments 1 to 4, wherein Y₂ is

“\*MERGEFORMAT\*MERGEFORMAT C₁₋₄alkyl.

Embodiment 6 is the compound of embodiment 5, wherein Y₂ is methyl.

Embodiment 7 is the compound of any one of embodiments 1 to 6, wherein
R₁₃ is selected from the group consisting of hydrogen, halo, C₁₋₆alkyl, C₁₋₆haloalkyl,
C₁₋₆hydroxyalkyl, C₁₋₆dihydroxyalkyl, C₃₋₈cycloalkyl, a 3- or 6-membered heterocyclyl having 1-3 heteroatoms as ring vertices selected from N, O and S; wherein heterocyclyl and cycloalkyl are substituted with 0 to 3 groups independently selected from the group consisting of C₁₋₄alkyl, —OH, —NH₂, —OR₂₁, halo, cyano and oxo.

Embodiment 8 is the compound of embodiment 7, wherein R₁₃ is selected from the group consisting of hydrogen, halo, C₁₋₆alkyl, and C₁₋₆haloalkyl.

Embodiment 9 is the compound of embodiment 8, wherein R₁₃ is hydrogen, Cl, Br, methyl, or CF₃.

Embodiment 10 is the compound of any one of embodiments 1 to 9, wherein
R₁ is selected from the group consisting of C₆₋₁₀aryl and a 5- to 9-membered heteroaryl group having 1 to 4 heteroatoms groups as ring vertices independently selected from N, C(O), O, and S; and is optionally substituted with 1 to 5 R₁₂ groups independently selected from the group consisting of halo, hydroxy, amino, C₁₋₄alkylamino, di(C₁₋₄alkyl)amino, cyano, C₁₋₄alkyl, C₁₋₄alkoxy, C₁₋₄hydroxyalkyl, C₁₋₄haloalkyl, C₁₋₄aminoalkyl, C₃₋₈cycloalkyl, C₃₋₈cycloalkenyl, NR₁₅C(O)R₁₄, NR₁₅C(O)OR₁₄, NR₁₄C(O)NR₁₅R₁₆, NR₁₅S(O)R₁₄, NR₁₅S(O)₂R₁₄, C(O)NR₁₅R₁₆, S(O)NR₁₅R₁₆, S(O)₂NR₁₅R₁₆, C(O)R₁₄, C(O)OR₁₄, OR₁₄, SR₁₄, S(O)R₁₄, and S(O)₂R₁₄.

Embodiment 11 is the compound of embodiment 10, wherein
R₁ is phenyl or a 5- to 6-member heteroaryl group having 1 to 4 heteroatoms as ring vertices independently selected from N, O, and S; and is optionally substituted with 1, 2 or 3 R₁₂ groups independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

Embodiment 12 is the compound of any one of embodiments 1 to 11, wherein R₂, R₃, R₄, R₅, R₈, R₉, R₁₀ and R₁₁ are hydrogen.

Embodiment 13 is the compound of any one of embodiments 1 to 12, wherein:
R₆ is selected from the group consisting of amino, C₁₋₄aminoalkyl, and C₁₋₄alkylamino; and
R₇ is selected from the group consisting of hydrogen, amido, cyano, halo, and hydroxy, or is selected from the group consisting of C₁₋₄alkyl, C₁₋₄hydroxyalkyl, C₃₋₆cycloalkyl, phenyl, and 5- or 6-membered heteroaryl, any of which is optionally substituted with one or two groups selected from the group consisting of amino, halo, hydroxy, cyano, trifluoromethyl, trifluoromethoxy, C₁₋₄alkyl, and C₁₋₄alkoxy.

Embodiment 14 is the compound of embodiment 13, wherein
R₆ is amino or aminomethyl; and
R₇ is selected from the group consisting of hydroxy, C₁₋₄alkyl, and C₁₋₄hydroxyalkyl.

Embodiment 15 is the compound of any one of embodiments 1 to 12, wherein
R₆ and R₇ together with the carbon atom to which they are both attached form a 3- to 7-membered saturated or unsaturated ring having 1 to 3 heteroatoms or groups as ring vertices independently selected from N, C(O), O, and S(O)ₘ, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

Embodiment 16 is the compound of embodiment 15, wherein
R₆ and R₇ together with the carbon atom to which they are both attached form a 4- to 6-membered saturated ring having 1 to 3 heteroatoms as ring vertices independently selected from N and O, and that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

Embodiment 17 is the compound of any one of embodiments 1 to 12, wherein
R₆ and R₇ together with the carbon atom to which they are both attached form a 3- to 7-membered cycloalkyl ring that is optionally substituted with one or two groups independently selected from the group consisting of amino, halo, hydroxy, C₁₋₄alkyl, C₁₋₄alkoxy, C₁₋₄alkylamino and C₁₋₄aminoalkyl.

Embodiment 18 is the compound of any one of embodiments 1 to 17, wherein
R₁ is selected from the group consisting of phenyl, pyridyl, pyrimidinyl, pyrazinyl, pyridazinyl, and 1,2,4-triazinyl; and is optionally substituted with 1, 2, or 3 R₁₂ independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, C₁₋₄aminoalkyl, and OR₁₄.

Embodiment 19 is the compound of embodiment 18, wherein R₁ is phenyl or pyridyl, each of which is substituted with 1 to 3 R₁₂.

Embodiment 20 is the compound of any one of embodiments 1 to 18, wherein R₁ is selected from the group consisting of:

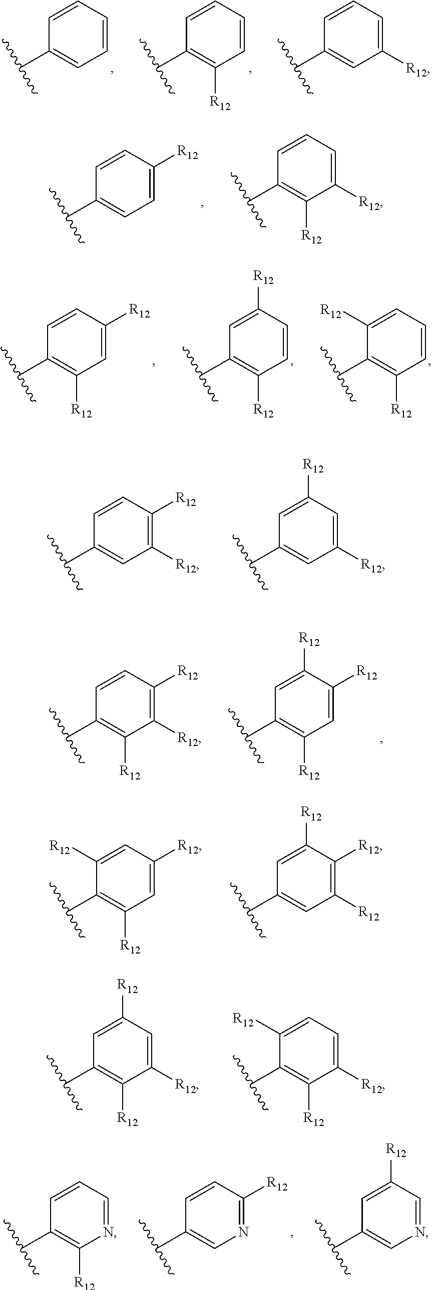

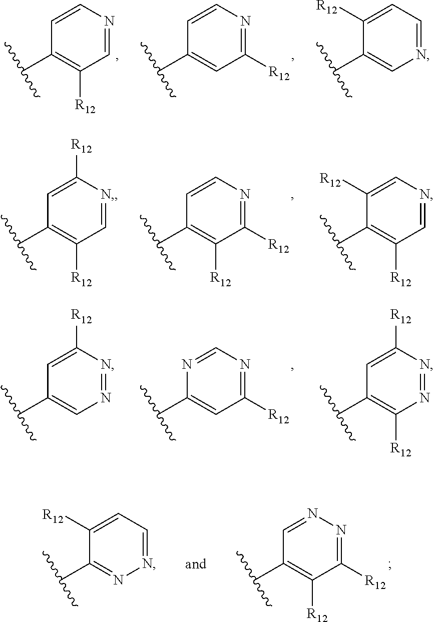

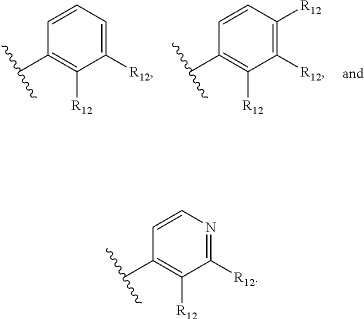

Embodiment 22 is the compound of any one of embodiments 1 to 21, wherein R₁₄ is selected from the group consisting of C₆₋₁₀aryl and a 5-10 membered heteroaryl, each of which is optionally substituted by one or more groups independently selected from the group consisting of halo, hydroxy, cyano, and C₁₋₄alkyl.

Embodiment 23 is the compound of embodiment 22, wherein R₁₄ is phenyl or a 5-6 membered heteroaryl having 1 to 4 heteroatoms as ring vertices independently selected from N, O, and S, each of which is optionally substituted by one or two groups independently selected from the group consisting of halo, hydroxy, cyano, and C₁₋₄alkyl.

Embodiment 24 is the compound of embodiment 1, wherein R₁₄ is selected from the group consisting of C₆₋₁₀aryl and a 5-10 membered heteroaryl, each of which is optionally substituted by one or more groups independently selected from the group consisting of

“\*MERGEFORMAT\*MERGEFORMAT C₁₋₄alkylamido, amino, halo, hydroxy, cyano, C₁₋₄alkyl, C₁₋₄alkoxy, C₁₋₄haloalkyl,

“\*MERGEFORMAT\*MERGEFORMAT C₁₋₄haloalkoxy, C₁₋₄alkylamino and C₁₋₄aminoalkyl.

Embodiment 25 is the compound of embodiment 1, wherein R₁ is

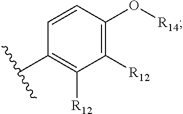

each R₁₂ is independently selected from the group consisting of halo, hydroxy, amino, C₁₋₄alkylamino, di(C₁₋₄alkyl)amino, C₁₋₄alkyl, C₁₋₄alkoxy, C₁₋₄haloalkyl, and C₁₋₄aminoalkyl; and
R₁₄ is phenyl or a 5-6 membered heteroaryl having 1 to 4 heteroatoms as ring vertices independently selected from N, O, and S, each of which is optionally substituted by one or two groups independently selected from the group consisting of C₁₋₄alkylamido, halo, hydroxy, cyano, and C₁₋₄alkyl.

Embodiment 26 is the compound of embodiment 1, wherein each R₁₂ is independently selected from the group consisting of F, Cl, Br, CH₃, OCH₃, CF₃,

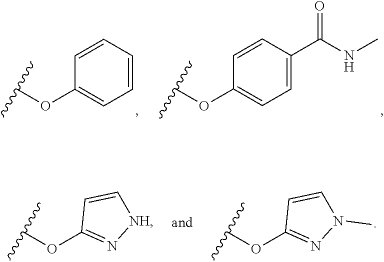

Embodiment 28 is a compound of embodiment 1 or 2, for use as a medicament.

Embodiment 29 is a compound of embodiment 1 or 2, for use in the treatment of a disease driven by one or more PTPN11 mutations.

Embodiment 30 is a compound of embodiment 1 or 2, for use in the treatment of cancer.

Embodiment 31 is the compound of embodiment 30, wherein the cancer is selected from the group consisting of leukemia, melanoma, breast cancer, and colon cancer.

Embodiment 32 is a compound of embodiment 1 or 2, for use in the manufacture of a medicament for the prevention or treatment of a disease or condition ameliorated by the inhibition of PTPN11.

Embodiment 33 is a pharmaceutical composition comprising a compound of embodiment 1 or 2, together with a pharmaceutically acceptable carrier.

Embodiment 34 is a method of inhibition of PTPN11 comprising contacting PTPN11 with a compound of embodiment 1 or 2.

Embodiment 35 is a method of treatment of a PTPN11-mediated disease comprising the administration of a therapeutically effective amount of a compound of embodiment 1 or 2, to a patient in need thereof.

Embodiment 36 is the method of embodiment 35, wherein the disease is cancer.

Embodiment 37 is the method of embodiment 36, wherein the cancer is selected from the group consisting of breast cancer, colon cancer, leukemia, and melanoma.

Embodiment 38 is a method of treatment of a PTPN11-mediated disease comprising the administration of:
a. a therapeutically effective amount of a compound of embodiment 1 or 2; and
b. another therapeutic agent.

Embodiment 39 is the method of embodiment 38, wherein the disease is cancer.

Embodiment 40 is the method of embodiment 39, wherein the cancer is selected from the group consisting of breast cancer, colon cancer, leukemia, and melanoma.

1-40. (canceled)

41. A mixture comprising
i) a first compound having the structure: 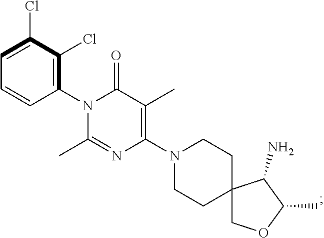
and
ii) a second compound having the structure: 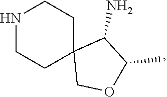
or a salt thereof.

42. The mixture of claim 41, wherein the mixture is a solution.

43. The mixture of claim 41, wherein mixture further comprises a third compound having the structure:

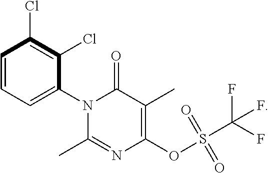

44. The mixture of claim 41, wherein mixture further comprises a third compound having the structure: 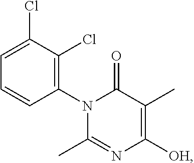 [SMILES: Cc1c(O)nc(C)n(-c2cccc(Cl)c2Cl)c1=O]
or a stereoisomer thereof.

45. A mixture, comprising:
i) a first compound having the structure: 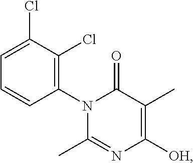 [SMILES: Cc1c(O)nc(C)n(-c2cccc(Cl)c2Cl)c1=O]
or a stereoisomer thereof; and
ii) a second compound having the structure: 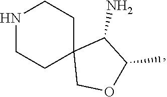
or a salt thereof.

46. A mixture, comprising:
i) a first compound having the structure: 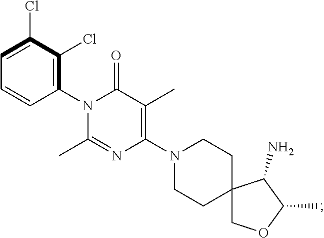
and
ii) a second compound having the structure: 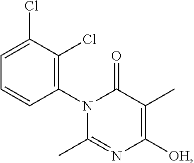 [SMILES: Cc1c(O)nc(C)n(-c2cccc(Cl)c2Cl)c1=O]
or a stereoisomer thereof.

47. A method, comprising:
a) separating the mixture of claim 41 into an organic layer and an aqueous layer; and
b) purifying the organic layer via silica gel chromatography to provide a solution comprising the first compound.

48. The method of claim 44, wherein the organic layer comprises an organic solvent.

49. The method of claim 48, wherein the organic solvent is dichloromethane.

50. The method of claim 47, further comprising c) performing chiral supercritical fluid chromatography using the solution to provide a purified solution comprising the first compound.
